(19) United States
(12) Patent Application Publication
(10) Pub. No.: US 2026/0124311 A1
(43) Pub. Date: May 7, 2026

(54) POLYMER-DRUG CONJUGATES FOR STING PATHWAY ACTIVATION

(71) Applicant: Vanderbilt University, Nashville, TN (US)

(72) Inventors: John T. Wilson, Nashville, TN (US); Karan Arora, Nashville, TN (US); Taylor L. Sheehy, Nashville, TN (US); Jacob Schulman, Nashville, TN (US)

(21) Appl. No.: 19/119,975

(22) PCT Filed: Oct. 12, 2023
(86) PCT No.: PCT/US2023/076732

§ 371 Date: Apr. 10, 2025

Related U.S. Application Data
(60) Provisional application No. 63/415,570, filed on Oct. 12, 2022.

(51) Int. Cl. A61K 47/60; A61K 47/02; A61K 47/26; A61P 37/04

(52) U.S. Cl. CPC A61K 47/60; A61K 47/02; A61K 47/26; A61P 37/04

(57) ABSTRACT

Disclosed herein are conjugates that can effectively deliver a STING agonist. An example conjugate includes a hydrophilic polymer, a diamidobenzimidazole (diABZI), and a linker attaching the diABZI to the hydrophilic polymer. Also disclosed are modified diABZI compounds and methods of making and using the conjugates.

### CROSS-REFERENCE TO RELATED APPLICATIONS

This application claims priority to U.S. Provisional Patent Application No. 63/415,570 filed on Oct. 12, 2022, which is incorporated fully herein by reference.

### FEDERALLY SPONSORED RESEARCH

This invention was made with government support under Grant No. R01 CA245134 awarded by the National Institutes of Health. The government has certain rights in the invention.

### TECHNICAL FIELD

This disclosure relates to polymer-STING agonist conjugates and their use in biomedical applications, such as drug delivery.

### INTRODUCTION

Stimulator of interferon genes (STING) is a receptor in the endoplasmic reticulum that propagates innate immune sensing of cytosolic pathogen-derived and self DNA. Compounds that can modulate STING (e.g., STING agonists) can be beneficial for numerous diseases, such as cancer. However, efficacy of STING agonists can be impeded by barriers to its delivery.

### SUMMARY

In one aspect, disclosed are conjugates, or a pharmaceutically acceptable salt thereof, comprising: a hydrophilic polymer; a diamidobenzimidazole (diABZI); and a linker attaching the diABZI to the hydrophilic polymer.

In another aspect, disclosed are pharmaceutical compositions comprising the disclosed conjugate, or a pharmaceutically acceptable salt thereof, and a pharmaceutically acceptable excipient.

In another aspect, disclosed are methods of modulating a STING pathway in a subject in need thereof, the method comprising administering to the subject an effective amount of the disclosed conjugate, or a pharmaceutically acceptable salt thereof, optionally in combination with a pharmaceutically acceptable excipient.

In another aspect, disclosed are methods of treating cancer in a subject in need thereof, the method comprising administering to the subject an effective amount of the disclosed conjugate, or a pharmaceutically acceptable salt thereof, optionally in combination with a pharmaceutically acceptable excipient, wherein the subject has melanoma, breast cancer, neuroblastoma, renal cell carcinoma, colon cancer, lung cancer, glioma, glioblastoma, pancreatic cancer, osteosarcoma, ovarian cancer, cervical cancer, bladder cancer, T and B cell lymphomas, medulloblastoma, head and neck cancer (HNSCC), liver cancer, or prostate cancer.

In another aspect, disclosed are compounds of formula (VI), or a pharmaceutically acceptable salt thereof,

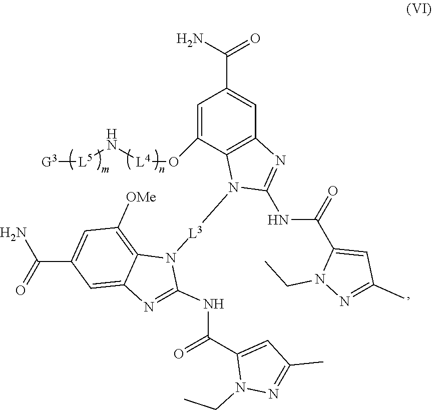

wherein: L³ is C₃₋₈alkylene or C₃₋₈alkenylene; L⁴ is C₁₋₆alkylene or C₁₋₆alkenylene; L⁵ is

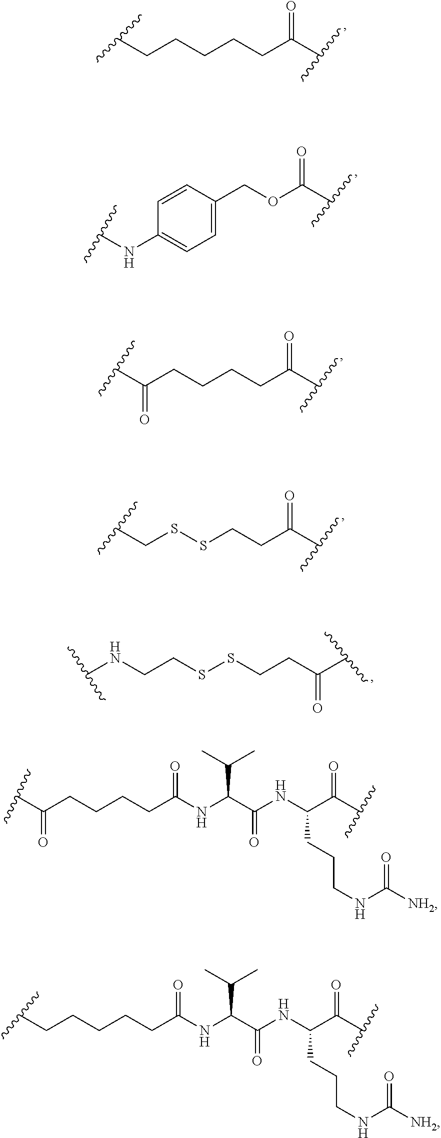

or a combination thereof; G³ is

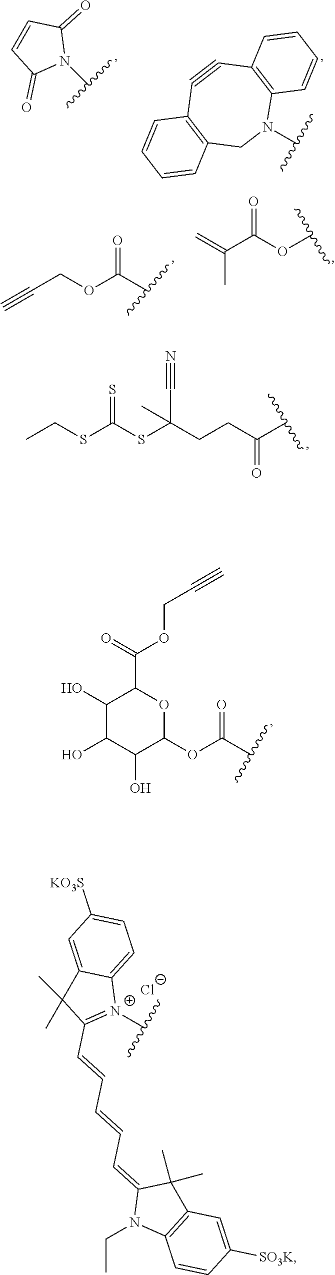

combination thereof; m is 0-4; and n is 1-2.

In another aspect, disclosed are pharmaceutical compositions comprising the disclosed compound, or a pharmaceutically acceptable salt thereof, and a pharmaceutically acceptable carrier.

In another aspect, disclosed are methods for treating a disease or disorder associated with STING dysfunction comprising administering to a subject in need thereof, a therapeutically effective amount of the disclosed compound, or a pharmaceutically acceptable salt thereof, or a pharmaceutical composition thereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A shows the isothermal calorimetry performed to determine K_{D} of binding of diamidobenzimidazole (diABZI)-amine (1) to human recombinant STING, using cyclic dinucleotides and commercially available diABZI as controls.

FIG. 1B shows the isothermal calorimetry performed to determine K_{D} of binding of diABZI-V/C-Mal (3) to human recombinant STING, using cyclic dinucleotides and commercially available diABZI as controls.

FIG. 2 shows the results from a Cathepsin-B release assay in conjunction with MALDI-MS analysis to determine cathepsin sensitivity of V/C linker compared to a non-cleavable conjugate.

FIG. 3A shows the dose-response curves from a THP1-Dual™ reporter assay comparing relative interferon production between diABZI-amine (1), diABZI-Mal (2), and diABZI-V/C-Mal (3).

FIG. 3B shows the dose-response curves from an Interferon-β sandwich ELISA performed on isolated splenocytes comparing relative interferon-β production between diABZI-Amine (1), diABZI-Mal (2) and diABZI-V/C-Mal (3).

FIG. 4A shows a comparison of STING activation between diABZI-amine (1) and diABZI-V/C-DBCO (9) in THP1 Dual™ reporter cells.

FIG. 4B shows a comparison of STING activation between diABZI-V/C-DBCO (9) and diABZI-DBCO (8) in THP1 Dual™ reporter cells.

FIG. 5 shows the dose-response curves for diABZI-ECT (10) and diABZI-SS-ECT (11) inducing IRF signaling in THP-1 Dual™ reporter cells at 24 hours.

FIG. 6 shows the dose-response curves diABZI-ECT (10), diABZI-Methacrylate (14), and diABZI-SS-methacrylate (15) inducing IRF signaling in THP-1 Dual Reporter cells at 24 hours.

FIG. 7A shows the isothermal calorimetry performed to determine K_{D} of binding of diABZI-20kPEG (20) to human recombinant STING, using cyclic dinucleotides and commercially available diABZI as controls.

FIG. 7B shows the isothermal calorimetry performed to determine K_{D} of binding of diABZI-V/C-20kPEG (21) to human recombinant STING, using cyclic dinucleotides and commercially available diABZI as controls.

FIG. 8 shows the results for a Cathepsin-B release assay in conjunction with MALDI-MS analysis to determine cathepsin sensitivity of example V/C linker compounds compared to the non-cleavable controls demonstrating the release of free drug with the expected compounds.

FIG. 9A shows the THP1-Dual™ reporter assay comparing relative Interferon-β production between cathepsin-cleavable PEG variants and diABZI-V/C-Mal.

FIG. 9B shows the THP1-Dual™ reporter assay comparing relative Interferon-β production between cleavable and non-cleavable diABZI PEG variants.

FIG. 9C shows the injection protocol in a B16 mouse tumor model.

FIG. 9D shows tumor growth curve over the course of treatment with diABZI-amine (1), diABZI-20kPEG (18), and diABZI-V/C-20kPEG (21).

FIG. 9E shows tumor volume of mice on 18 days post inoculation (end of study) for diABZI-amine (1), diABZI-20kPEG (18), and diABZI-V/C-20kPEG (21). *P<0.05, **P<0.01, ***P<0.001, ****P<0.0001; one-way ANOVA with Tukey's multiple comparisons test.

FIG. 10A shows the dose-response curves from the THP1-Dual™ reporter assay comparing relative Interferon-β production of cleavage diABZI Beta-Glu variants with different sized spacers.

FIG. 10B shows the dose-response curves from the THP1-Dual™ reporter assay comparing relative Interferon-β production of non-cleavage diABZI Beta-Glu variants with different sized spacers.

FIG. 11 shows the gel permeation chromatography (GPC) elution peaks and results of 25 kDa DMA-co-AzEMA (26) and 100 kDa DMA-co-AzEMA (27).

FIG. 12A shows the UV-Vis characterization of a DBCO-V/C-diABZI polymer drug conjugate labeled with fluorescent Cy5 dye.

FIG. 12B shows the UV-Vis characterization of an unlabeled DBCO-V/C-diABZI polymer drug conjugate.

FIG. 12C shows the identification of the bleed-over absorbance (A_{bleed-over}) for Cy5-labeled polymer and for Cy5-labeled polymer post diABZI conjugation.

FIG. 13 shows the dynamic light scattering (DLS) graphs of DMA-g-V/C-diABZI polymer drug conjugates (28) and (29) indicating no uniform particle formation.

FIG. 14A shows the dose-response curves for STING activation via INF production for diABZI-VC-DBCO (9) and DMA-g-V/C-diABZI polymer drug conjugates (28) and (29) measured with THP1 Dual™ reporter cells.

FIG. 14B shows the dose-response curve for the Interferon-β sandwich ELISA performed on isolated splenocytes comparing relative Interferon-β production between diABZI-VC-DBCO (9) and DMA-g-V/C-diABZI polymer drug conjugates (28) and (29).

FIG. 15A shows the dose-response curve of DMA-g-V/C-diABZI (28) and (29) and DMA-g-V/C-diABZI polymer drug conjugates (30) and (31) induction of STING activation in THP-1 Dual™ reporter cells.

FIG. 15B shows the dose-response curve of DMA-g-V/C-diABZI (28) and (29) and DMA-g-V/C-diABZI polymer drug conjugates (30) and (31) Interferon-β production using an Interferon-β sandwich ELISA and isolated splenocytes.

FIG. 16A shows the pharmacokinetic profile of 100 kDa and 25 kDa carriers. A half-life of 1.1 and 4.4 hours was determined for 25 kDa DMA-g-V/C-diABZI (28) and 100 kDa DMA-g-V/C-diABZI (29), respectively.

FIG. 16B shows the biodistribution of 25 kDa DMA-g-V/C-diABZI (28) and 100 kDa DMA-g-V/C-diABZI (29) in E0771 tumor bearing mice comparing relative blood fluorescence in organs and tumors 24 hours post inoculation.

FIG. 16C shows the distribution of 100 kDa DMA-g-V/C-diABZI (29) across organs and tumors in healthy E0771 mice. *p≤0.05, **p≤0.01, ***p≤0.001, ****p≤0.0001.

FIG. 16D shows the distribution of 25 kDa DMA-g-V/C-diABZI (28) across organs and tumors in healthy E0771 breast tumor bearing mice. *p≤0.05, **p≤0.01, ***p≤0.001, ****p≤0.0001.

FIG. 16E shows the distribution of 25 kDa DMA-g-V/C-diABZI (28) and 100 kDa DMA-g-V/C-diABZI (29) in the liver and kidney 24 hours post inoculation in E0771 breast tumor bearing mice. *p≤0.05, **p≤0.01, ***p≤0.001, ****p≤0.0001.

FIG. 16F shows the distribution of 25 kDa DMA-g-V/C-diABZI (28) and 100 kDa DMA-g-V/C-diABZI (29) in the tumor and kidney 24 hours post inoculation in E0771 breast tumor bearing mice. *p≤0.05, **p≤0.01, ***p≤0.001, ****p≤0.0001.

FIG. 16G shows the distribution of 25 kDa DMA-g-V/C-diABZI (28) and 100 kDa DMA-g-V/C-diABZI (29) in the tumor and liver 24 hours post inoculation in E0771 breast tumor bearing mice. *p<0.05, **p<0.01, ***p<0.001, ****p<0.0001.

FIG. 17A shows the weight loss of E0771 breast tumor bearing mice in response to diABZI-V/C-DBCO (9), 25 kDa DMA-g-V/C-diABZI (28), and 100 kDa DMA-g-V/C-diABZI (29).

FIG. 17B shows the tumor volume of E0771 breast tumor bearing mice in response to diABZI-V/C-DBCO (9), 25 kDa DMA-g-V/C-diABZI (28), and 100 kDa DMA-g-V/C-diABZI (29). *p≤0.05, **p≤0.01, ***p≤0.001, ****p≤0.0001.

FIG. 17C shows the survival probability of E0771 breast tumor bearing mice in response to diABZI-V/C-DBCO (9), 25 kDa DMA-g-V/C-diABZI (28), and 100 kDa DMA-g-V/C-diABZI (29). *p≤0.05, **p≤0.01, ***p≤0.001, ****p<0.0001.

FIG. 18A shows dose-response curves for diABZI-Amine (1), diABZI-ECT (10), 25 kDa diABZI-DMA (32), 50 kDa diABZI-DMA(33), and 175 kDa diABZI-DMA(34) induction of IRF signaling in THP-1 Dual™ reporter cells.

FIG. 18B shows dose-response curves for diABZI-ECT (10), 50 kDa diABZI-DMA (33), diABZI-SS-ECT (11) and 50 kDa diABZI-SS-DMA (35) induction of IRF signaling in THP-1 Dual™ reporter cells.

FIG. 19A shows dose-response curves for type-I interferon (INF) activation by diABZI-ECT (10) and 50 kDa DMA-diABZI (33) measured with a the THP-1 Dual™ reporter assay.

FIG. 19B shows dose-response curves for NF-kb activation by diABZI-ECT (10) and 50 kDa DMA-diABZI (33) measured with a the THP-1 Dual™ reporter assay.

FIG. 19C shows dose-response curves for INF activation by diABZI-ECT (10) and 50 kDa DMA-diABZI (33) in a THP-1 Dual™ reporter assay with STING knock-out (KO) cells.

FIG. 19D shows dose-response curves for NF-kb activation by diABZI-ECT (10) and 50 kDa DMA-diABZI (33) in a THP-1 Dual™ reporter assay with STING knock-out (KO) cells.

FIG. 20A shows the kinetic profile of diABZI-ECT (10) and 50 kDa DMA-diABZI (33) determined by PCR analysis of Interferon-β.

FIG. 20B shows the kinetic profile of diABZI-ECT (10) and 50 kDa DMA-diABZI (33) determined by PCR analysis of CXCL10.

FIG. 20C shows the kinetic profile of diABZI-ECT (10) and 50 kDa DMA-diABZI (33) determined by PCR analysis of TNF.

FIG. 20D shows the kinetic profile of diABZI-ECT (10) and 50 kDa DMA-diABZI (33) determined by PCR analysis of IL-6.

FIG. 21 shows the confocal Microscopy of STING dynamics dependent on activation via diABZI-ECT (10) and 50 kDa DMA-diABZI (33) utilizing GFP-STING expressing murine embryonic fibroblasts.

FIG. 22A shows the cellular uptake of sCy5-labeled diABZI (16) at 37° C. and 4° C. in THP-1 Dual™ cells.

FIG. 22B shows the cellular uptake of sCy5-labeled DMA-diABZI (37) at 37° C. and 4° C. in THP-1 Dual™ cells.

### DETAILED DESCRIPTION

The ability to effectively deliver STING agonists, such as diABZI, in vivo presents a number of challenges. For example, STING agonists require a half-life that is long enough to be effective, but also short enough to avoid adverse side effects. Unfortunately, STING agonists have pharmacokinetics and pharmacodynamics that are difficult to predict and do not follow patterns seen with chemotherapeutics. In addition, STING agonists typically require additional immunomodulators (e.g., immune checkpoint inhibitors) to be effective. However, disclosed herein, are diABZI conjugates that take advantage of the disclosed linkers and hydrophilic polymers to provide an effective in vivo delivery. Furthermore, the disclosed diABZI conjugates are effective without additional immunomodulators. The efficacy of the disclosed diABZI conjugates was demonstrated in a breast cancer tumor model where the disclosed diABZI conjugates significantly improved therapeutic response compared to free diABZI as measured by weight loss, tumor regression, and survival.

### 1. DEFINITIONS

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3_{rd} Edition, Cambridge University Press, Cambridge, 1987; the entire contents of each of which are incorporated herein by reference.

The term “alkyl,” as used herein, refers to a straight or branched, saturated hydrocarbon chain containing from 1 to 30 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and n-dodecyl. The alkyl group may be substituted or unsubstituted.

The term “alkylene,” as used herein, refers to a divalent alkyl group, examples of which include, but are not limited to, —CH₂CH₂—, —CH₂CH₂CH₂—, —CH₂CH₂CH₂CH₂—, and —CH₂CH₂CH₂CH₂CH₂—. An alkylene group may be optionally substituted with one or more substituents.

The term “alkenylene,” as used herein, refers to a divalent alkenyl group, examples of which include, but are not limited to —CH═CH—, —CH═CH—CH₂—, —CH═CH—CH₂—CH₂— and —CH₂—CH═CH—CH₂—. An alkenylene group may be optionally substituted with one or more substituents.

The term “alkynylene” refers to a divalent alkynyl group, examples of which include, but are not limited to —C≡C—, —C≡C—CH₂—, —C≡C—CH₂—CH₂— and —CH₂—C≡C—CH₂—. An alkynylene group may be optionally substituted with one or more substituents.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The term “effective dosage” or “therapeutic dosage” or “therapeutically effective amount” or “effective amount,” as used herein, refers to an amount sufficient to effect beneficial or desirable biological and/or clinical results, to modulate a biological process, and/or treat a disease or one or more of its symptoms and/or to prevent or reduce the risk of the occurrence or reoccurrence of the disease or disorder or symptom(s) thereof. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result.

Typically, since a prophylactic dose is used in subjects prior to or at an earlier stage of disease, the prophylactically effective amount will be less than the therapeutically effective amount. In reference to STING pathway modulation and/or cancer treatment an effective or therapeutically effective amount can include an amount sufficient to, among other things, increase a cytokine serum level or improve survival from a disease, such as cancer.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

Terms such as “alkyl,” “cycloalkyl,” “alkylene,” etc. may be preceded by a designation indicating the number of atoms present in the group in a particular instance (e.g., “C₁₋₄alkyl,” “C₃₋₆ cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### 2. CONJUGATES

Provided herein are conjugates that can safely and effectively deliver a STING agonist. The conjugate includes a hydrophilic polymer, a diamidobenzimidazole (diABZI), and a linker attaching, e.g., covalently, the diABZI to the hydrophilic polymer. The hydrophilic polymer and the linker of the conjugate can solubilize diABZI. Accordingly, the conjugate itself can have a useful aqueous solubility. For example, the conjugate can have an aqueous solubility of greater than or equal to 1 mg/ml, greater than or equal to 50 mg/ml, greater than or equal to 100 mg/ml, greater than or equal to 200 mg/ml, greater than or equal to 300 mg/ml, greater than or equal to 400 mg/ml, or greater than or equal to 500 mg/ml. In some embodiments, the conjugate has an aqueous solubility of less than or equal to 500 mg/ml, less than or equal to 400 mg/ml, less than or equal to 300 mg/ml, less than or equal to 200 mg/ml, less than or equal to 100 mg/ml, or less than or equal to 50 mg/ml. In some embodiments, the conjugate has an aqueous solubility of about 1 mg/ml to about 500 mg/ml, such as about 1 mg/ml to about 200 mg/ml, about 10 mg/ml to about 100 mg/ml, or about 50 mg/ml to about 500 mg/ml. Aqueous solubility of the conjugate can be measured by techniques known within the art such as measuring how many grams of the conjugate can be dissolved in a specified volume of water.

The conjugate can include a varying number of diABZI. For example, the conjugate can include 1 to 15 diABZI per hydrophilic polymer, such as 1 to 12 diABZI per hydrophilic polymer, 1 to 10 diABZI per hydrophilic polymer, 1 to 8 diABZI per hydrophilic polymer, 1 to 6 diABZI per hydrophilic polymer, 1 to 4 diABZI per hydrophilic polymer, 1 to 2 diABZI per hydrophilic polymer, 2 to 10 diABZI per hydrophilic polymer, or 2 to 8 diABZI per hydrophilic polymer. In some embodiments, the conjugate includes greater than or equal to 1 diABZI per hydrophilic polymer, greater than or equal to 2 diABZI per hydrophilic polymer, greater than or equal to 3 diABZI per hydrophilic polymer, greater than or equal to 4 diABZI per hydrophilic polymer, or greater than or equal to 5 diABZI per hydrophilic polymer. In some embodiments, the conjugate includes less than or equal to 15 diABZI per hydrophilic polymer, less than or equal to 12 diABZI per hydrophilic polymer, less than or equal to 10 diABZI per hydrophilic polymer, less than or equal to 8 diABZI per hydrophilic polymer, or less than or equal to 6 diABZI per hydrophilic polymer. The number of diABZI per hydrophilic polymer can be modulated by altering the structure of the hydrophilic polymer, the linker, diABZI, or a combination thereof. For example, the hydrophilic polymer can have a specific number of reactive moieties that can be used to attach the diABZI. The number of diABZI per conjugate can be measured by techniques known within the art such as nuclear magnetic resonance (NMR), fluorescence, and/or Ultraviolet-visible (UV-Vis) spectroscopy.

The disclosed conjugates may exist as salts, such as pharmaceutically acceptable salts. The term “pharmaceutically acceptable salt” refers to salts or zwitterions of the conjugates which are water or oil-soluble or dispersible, suitable for administration to a subject (e.g., treatment of disorders) without undue toxicity, irritation, and allergic response, commensurate with a reasonable benefit/risk ratio and effective for their intended use. The salts may be prepared during the final isolation and purification of the conjugates or separately by reacting an amino group of the conjugates with a suitable acid. For example, the conjugate may be dissolved in a suitable solvent and treated with at least one equivalent of an acid, like hydrochloric acid. The resulting salt may precipitate out and be isolated by filtration and dried under reduced pressure.

Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. Amino groups of the conjugates may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed conjugates by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### A. Hydrophilic Polymers

The hydrophilic polymer can aid in solubilizing the diABZI and can aid the overall delivery properties of the conjugate. The hydrophilic polymer can be any suitable polymer having a hydrophilic character (e.g., having aqueous solubility) and having the ability to attach (e.g., covalently) to the diABZI through the linker. Example hydrophilic polymers include, but are not limited to, polyethylene glycol (PEG), a copolymer comprising a recurring hydrophilic unit and a recurring linking unit, poly(dimethylacrylamide) (PDMA), dextran, poly(lysine), poly(arginine), poly(glutamic acid), poly(aspartic acid), polyethylenimine (PEI), poly(mannose MA), poly(glucose MA), poly(galactose MA), poly(methacrylic acid), poly(propyl acrylic acid), a zwitterionic polymer, poly(phosphorylcholine), poly(sulfobetaine), poly(carboxylbetaine), poly(2-methacryloyloxyethyl phosphorylcholine), and combinations thereof.

In some embodiments, the hydrophilic polymer includes PEG, a copolymer comprising a recurring hydrophilic unit and a recurring linking unit, PDMA, dextran, poly(propyl acrylic acid), a polyamino acid (e.g., poly(lysine)), a zwitterionic polymer, or a combination thereof. In some embodiments, the hydrophilic polymer includes PEG, a copolymer comprising a recurring hydrophilic unit and a recurring linking unit, PDMA, dextran, a polyamino acid (e.g., poly(lysine)), or a combination thereof. In some embodiments, the hydrophilic polymer includes PEG, a copolymer comprising a recurring hydrophilic unit and a recurring linking unit, or PDMA.

The hydrophilic polymer can solubilize diABZI due to its useful aqueous solubility. In some embodiments, the hydrophilic polymer has an aqueous solubility of greater than or equal to 1 mg/ml, greater than or equal to 50 mg/ml, greater than or equal to 100 mg/ml, greater than or equal to 200 mg/ml, greater than or equal to 300 mg/ml, greater than or equal to 400 mg/ml, or greater than or equal to 500 mg/ml. In some embodiments, the hydrophilic polymer has an aqueous solubility of less than or equal to 500 mg/ml, less than or equal to 400 mg/ml, less than or equal to 300 mg/ml, less than or equal to 200 mg/ml, less than or equal to 100 mg/ml, or less than or equal to 50 mg/ml. In some embodiments, the hydrophilic polymer has an aqueous solubility of about 1 mg/ml to about 500 mg/ml, such as about 1 mg/ml to about 200 mg/ml, about 10 mg/ml to about 100 mg/ml, or about 50 mg/ml to about 500 mg/ml. Aqueous solubility of the hydrophilic polymer can be measured by techniques known within the art such as measuring how many grams of the hydrophilic polymer can be dissolved in a specified volume of water.

The hydrophilic polymer can have a varying molecular weight. For example, the hydrophilic polymer can have a number average molecular weight of about 1 kDa to about 200 kDa, such as about 1 kDa to about 185 kDa, about 2 kDa to about 180 kDa, about 2 kDa to about 175 kDa, about 10 kDa to about 150 kDa, about 15 kDa to about 120 kDa, about 1 kDa to about 150 kDa, about 2 kDa to about 100 kDa, about 1 kDa to about 110 kDa, about 50 kDa to about 200 kDa, or about 75 kDa to about 200 kDa. In some embodiments, the hydrophilic polymer has a number average molecular weight of greater than 1 kDa, greater than 2 kDa, greater than 5 kDa, greater than 10 kDa, greater than 25 kDa, or greater than 50 kDa. In some embodiments, the hydrophilic polymer has a number average molecular weight of less than 200 kDa, less than 185 kDa, less than 180 kDa, less than 175 kDa, less than 150 kDa, less than 125 kDa, less than 110 kDa, or less than 100 kDa.

The term “molecular weight” in relation to the hydrophilic polymer refers to number average molecular weight (Mₙ) unless noted otherwise. Molecular weight can be measured by standard techniques known within the art, such as gel permeation chromatography, size exclusion chromatography, NMR, and/or rheological analysis. In some embodiments, the polymer's Mₙ is measured by gel permeation chromatography.

The hydrophilic polymer may be commercially available or can be synthesized via polymerization techniques known within the art. For example, in some embodiments, the hydrophilic polymer, such as the copolymer and/or PDMA, is synthesized via reversible addition-fragmentation chain-transfer (RAFT) polymerization.

The hydrophilic polymer can include PEG. PEG can have a molecular weight as described herein for the hydrophilic polymer. In some embodiments, PEG has a number average molecular weight of about 1 kDa to about 50 kDa, such as about 2 kDa to about 40 kDa, about 1.5 kDa to about 30 kDa, or about 2 kDa to about 20 kDa. In some embodiments, PEG has a number average molecular weight of greater than 1 kDa, greater than 1.5 kDa, greater than 2 kDa, or greater than 5 kDa. In some embodiments, PEG has a number average molecular weight of less than 50 kDa, less than 45 kDa, less than 40 kDa, less than 35 kDa, less than 30 kDa, or less than 25 kDa.

The hydrophilic polymer can include a copolymer including a recurring hydrophilic unit and a recurring linking unit. The hydrophilic unit can be derived from any suitable hydrophilic monomer that, e.g., can be included in a RAFT polymerization. Example hydrophilic units include, but are not limited to, a dimethylacrylamide unit, a (2-diethylamino) ethyl methacrylate (DEAEMA) unit, a (2-dimethylamino) ethyl methacrylate (DMAEMA) unit, a N-(2-hydroxypropyl)methacrylamide unit, a 2-amino methacrylate hydrochloride unit, a polyethylene glycol methacrylate (PEGMA) unit, and combinations thereof. In some embodiments, the recurring hydrophilic unit of the copolymer includes a DMA unit, a DEAEMA unit, a DMAEMA unit, a PEGMA unit, or a combination thereof. In some embodiments, the recurring hydrophilic unit of the copolymer includes a DMA unit, a PEGMA unit, or a combination thereof. Unit as used herein, refers to a monomeric unit that is the resultant unit included in the polymer when a monomer is polymerized. For example, when a dimethylacrylamide monomer is used as part of the polymerization, the resultant monomeric unit included in the polymer is a dimethylacrylamide unit.

The copolymer can have a molecular weight as described herein for the hydrophilic polymer. In some embodiments, the copolymer has a number average molecular weight of about 15 kDa to about 150 kDa, such as about 20 kDa to about 125 kDa, about 25 kDa to about 100 kDa, or about 15 kDa to about 110 kDa. In some embodiments, the copolymer has a number average molecular weight of greater than 10 kDa, greater than 15 kDa, greater than 20 kDa, or greater than 50 kDa. In some embodiments, the copolymer has a number average molecular weight of less than 150 kDa, less than 125 kDa, less than 110 kDa, or less than 100 kDa.

In some embodiments, the recurring hydrophilic units of the copolymer have formula (1)

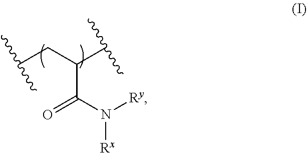

wherein R^{x} and R^{y} are each independently hydrogen or C₁₋₄alkyl. In some embodiments, R^{x} and R^{y} are each methyl.

The linking unit of the copolymer can be used to attach the diABZi to the copolymer. Accordingly, the linking unit of the copolymer can include the linker as described herein. The copolymer may include linking units not attached to diABZI. For example, the copolymer may include linking units to react with other molecules other than diABZI. In addition, the diABZI can be attached to the polymer through the use of a chain transfer agent as described herein. In some embodiments, the recurring linking units have formula (II)

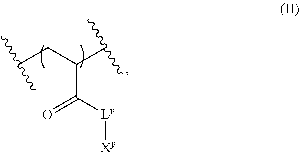

wherein L^{y} is —C₁₋₄alkylene-, —C₁₋₄alkenylene-, or —C₁₋₁₄alkynylene-, and X^{y} is azide, alkyne, amine, maleimide, or cyclooctyne. In some embodiments, L^{y} is —C₁₋₄alkylene-. In some embodiments, X^{Y} is azide.

The copolymer may also include further recurring units that are derived from monomers suitable for RAFT polymerization, which can be seen in the Examples below.

The hydrophilic polymer can include PDMA (e.g., homopolymer of DMA). PDMA can have a molecular weight as described herein for the hydrophilic polymer. In some embodiments, PDMA has a number average molecular weight of about 15 kDa to about 185 kDa, such as about 20 kDa to about 180 kDa, or about 25 kDa to about 175 kDa. In some embodiments, PDMA has a number average molecular weight of greater than 15 kDa, greater than 20 kDa, or greater than 25 kDa. In some embodiments, PDMA has a number average molecular weight of less than 185 kDa, less than 180 kDa, or less than 175 kDa.

### B. Diamidobenzimidazoles

DiABZI is a non-nucleotide based STING agonist. DiABZI has a core diamidobenzimidazole structure and can be modified at different substituents. Description of different diABZI structures can be found in Xi et al., Design, Synthesis, and Biological Evaluation of Amidobenzimidazole Derivatives as Stimulator of Interferon Genes (STING) Receptor Agonists, J Med Chem, 2020 Jan. 9; 63(1):260-282; Jeon et al., Development of Potent Immune Modulators Targeting Stimulator of Interferon Genes Receptor, J Med Chem, 2022 Apr. 14; 65(7):5407-5432; Song et al., Structure-Activity Relationship Study of Amidobenzimidazole Analogues Leading to Potent and Systemically Administrable Stimulator of Interferon Gene (STING) Agonists, 2021 Feb. 11; 64(3):1649-1669—all of which are incorporated by reference herein in their entirety.

In some embodiments, diABZI has formula (III)

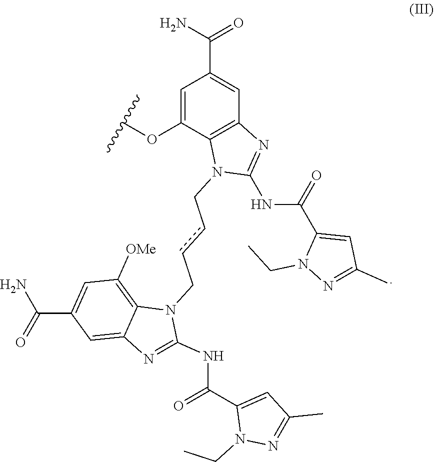

The [symbol] refers to the presence of a single or a double bond.

### C. Linkers

The linker attaches diABZI to the hydrophilic polymer. The linker can be a cleavable linker or a non-cleavable linker. Cleavable linkers are linkers that can be degraded by or in a biological environment in a biologically relevant time scale. For example, cleavable linkers include, but are not limited to, hypoxia sensitive linkers, reactive oxygen species (ROS) sensitive linkers, pH sensitive linkers, redox sensitive linkers, enzyme sensitive linkers, light sensitive linkers, and those degraded by hydrolysis. In contrast, non-cleavable linkers are linkers that are not degraded by or in a biological environment in a biologically relevant time scale, such as amide-based linkers and carbamate-based linkers.

In some embodiments, the linker comprises a hypoxia sensitive linker, a reactive oxygen species (ROS) sensitive linker, a pH sensitive linker, a redox sensitive linker, an enzyme sensitive linker, a light sensitive linker, or a combination thereof.

In some embodiments, the enzyme sensitive linker is a matrix metalloproteinase sensitive linker, a p-aminobenzyl alcohol system linker, a cathepsin sensitive linker, a beta-glucuronidase sensitive linker, an esterase sensitive linker, or a combination thereof. In some embodiments, the enzyme sensitive linker comprises

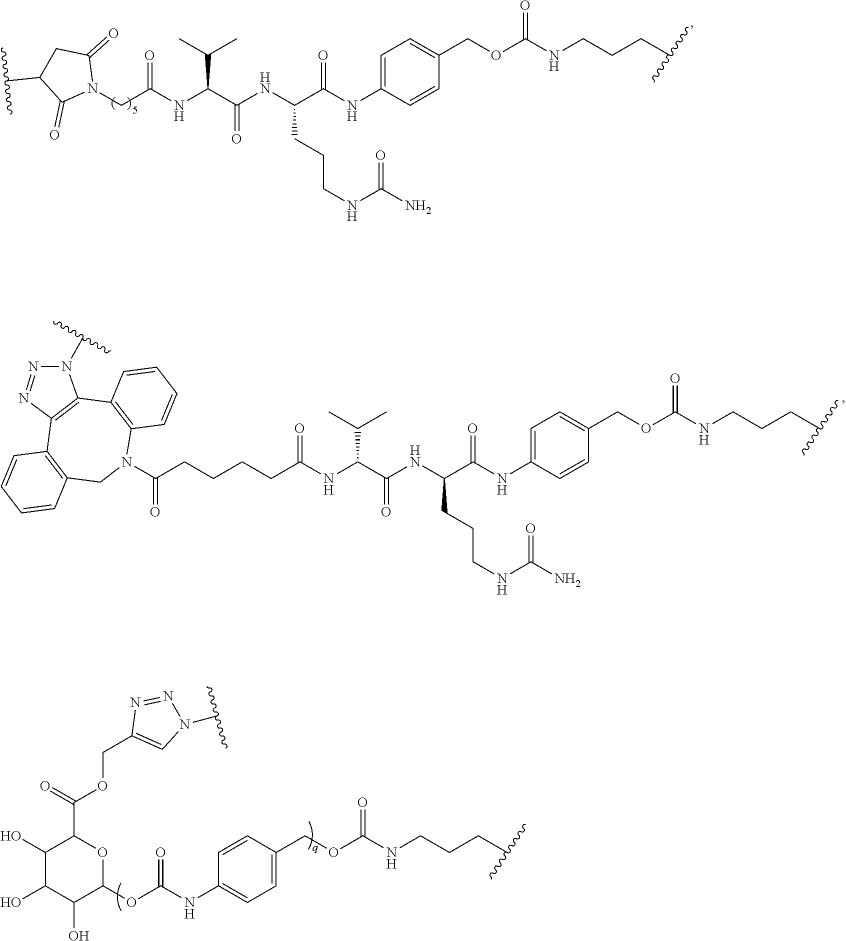

wherein q is 1 to 3,

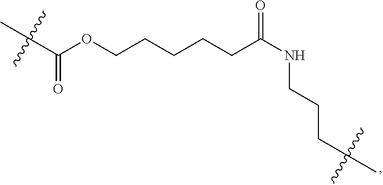 [SMILES: *CCCNC(=O)CCCCCOC(*)=O]

or a combination thereof.

In some embodiments, the redox sensitive linker is a glutathione sensitive linker, a nitroreductase/NADH sensitive linker, or a combination thereof. In some embodiments, the redox sensitive linker comprises a disulfide linkage. In some embodiments, the redox sensitive linker comprises

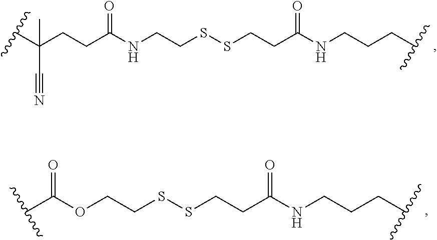 [SMILES: *CCCNC(=O)CCSSCCNC(=O)CCC(*)(C)C#N ; *CCCNC(=O)CCSSCCOC(*)=O]

or a combination thereof.

In some embodiments, the pH sensitive linker is a hydrazone, a silyl ether, a low pH sensitive linker, or a combination thereof. In some embodiments, the hypoxia sensitive linker is azobenzene.

Even though the linker can be non-cleavable, it has been found that such a linker can still provide an active diABZI. In some embodiments, the linker is non-cleavable and comprises

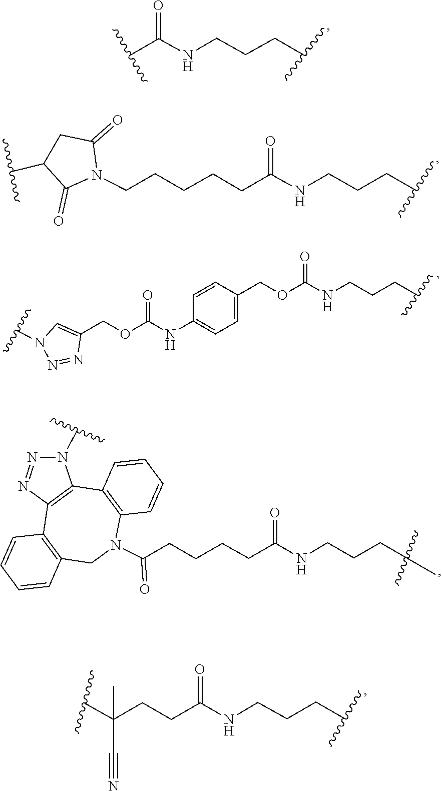

or a combination thereof.

In some embodiments, the linker includes a linking moiety and a spacer, the spacer being attached to the diABZI and the linking moiety being attached to the hydrophilic polymer. Further description of the linking moiety and the spacer can be seen below.

### D. Synthesis of Conjugates

Also provided herein are method of synthesizing the conjugates. DiABZI can be attached to the hydrophilic polymer through the linker either during the polymerization or post-polymerization via, e.g., conjugating with reactive groups present on the hydrophilic polymer, diABZI, or both.

In some embodiments, diABZI is included as part of the polymerization of the hydrophilic polymer as, e.g., a diABZI acrylate-based monomer or a diABZI chain transfer agent.

During the polymerization, the diABZI acrylate-based monomer can be polymerized and attached to the polymer. Likewise, diABZI can be used as a chain transfer agent and attached to the hydrophilic polymer during synthesis of the hydrophilic polymer.

In some embodiments, diABZI is post-synthetically attached to the hydrophilic polymer. For example, the diABZI and the hydrophilic polymer may each individually have functional groups that are complimentary to each other in that they can form a covalent bond between the functional groups under appropriate conditions. In these embodiments, what results in the linker, e.g., attaching the diABZI to the hydrophilic polymer, can be included as part of the diABZI, as part of the hydrophilic polymer, or both. After the conjugation occurs, the linker attaches the diABZI to the hydrophilic polymer.

Representative complimentary functional groups that can form a covalent bond include, but are not limited to, an amine and an activated ester, an amine and an isocyanate, an amine and an isothiocyanate, an amine and a carbonate, thiols for formation of disulfides, an aldehyde and amine for enamine formation, and an azide for formation of an amide via a Staudinger ligation. Functional groups suitable for conjugation also include bioorthogonal functional groups. Bioorthogonal functional groups can selectively react with a complementary bioorthogonal functional group. Bioorthogonal functional groups include, but are not limited to, an azide and alkyne for formation of a triazole via Click-chemistry reactions, trans-cyclooctene (TCO) and tetrazine (Tz) (e.g., 1,2,4,5-tetrazine), and others. Depending on the functional groups, different bonds or linkages can be formed between diABZI and the hydrophilic polymer.

### E. Example Conjugates

In some embodiments, the conjugate includes a cleavable linker and has formula (IV)

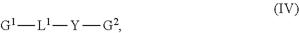

wherein: G¹ is the hydrophilic polymer; G² is diABZI; Y is a spacer selected from the group consisting of —NR^{a}—C₁₋₁₀alkylene- and —NR^{a}—C₁₋₁₀alkenylene-; L¹ is a linking moiety selected from the group consisting of

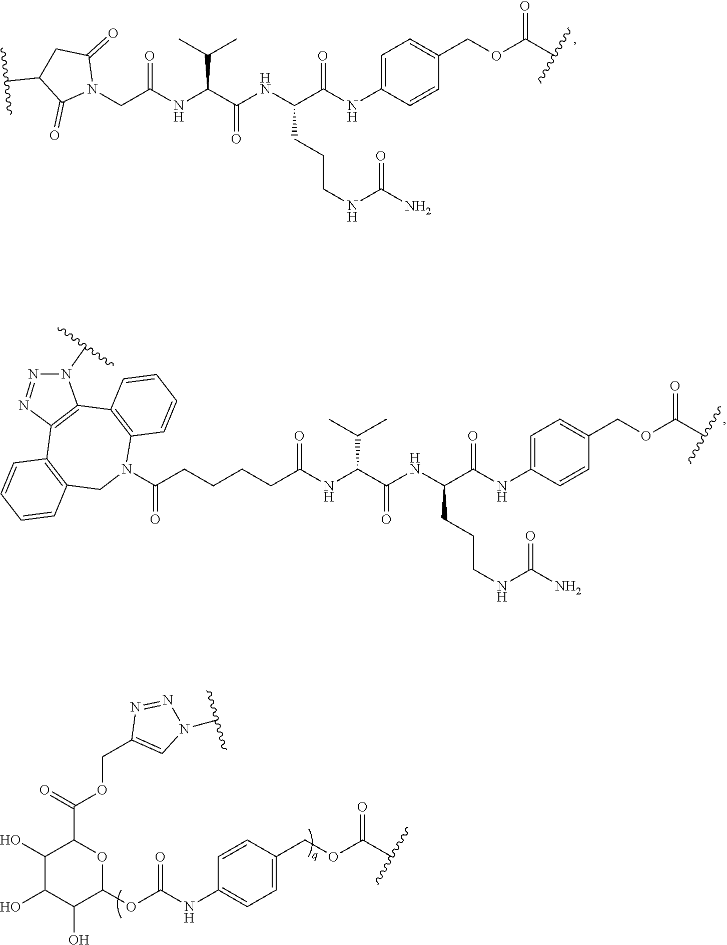

wherein q is 1 to 3,

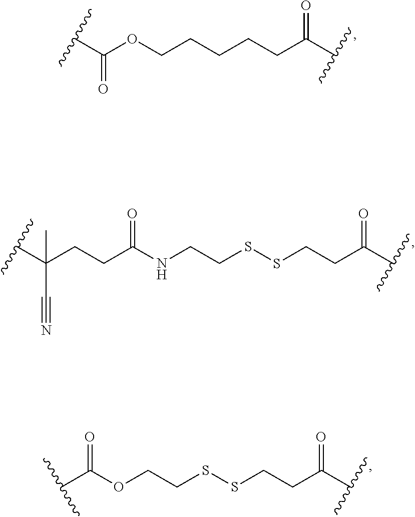 [SMILES: *C(=O)CCCCCOC(*)=O ; *C(=O)CCSSCCNC(=O)CCC(*)(C)C#N ; *C(=O)CCSSCCOC(*)=O]

and a combination thereof; and
R^{a} is hydrogen, C₁₋₆alkyl, or C₁₋₆haloalkyl.

In some embodiments, the conjugate includes a non-cleavable linker and has formula (V)

G¹-L¹-Y-G² (V),

wherein: G¹ is the hydrophilic polymer; G² is diABZI; Y is a spacer selected from the group consisting of —NR^{a}—C₁₋₁₀alkylene- and —NR^{a}—C₁₋₁₀alkenylene-; L² is a linking moiety selected from the group consisting of

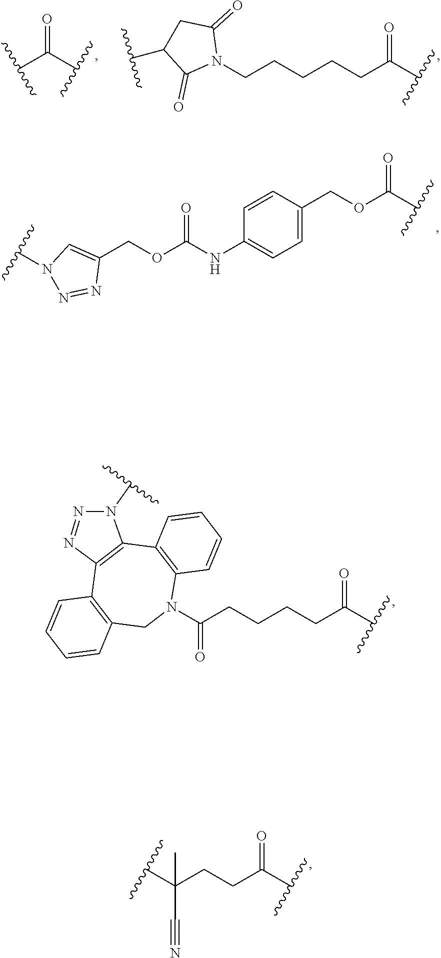

and a combination thereof; and
R^{a} is hydrogen, C₁₋₆alkyl, or C₁₋₆haloalkyl.

### 3. USES OF THE CONJUGATES

### A. Pharmaceutical Compositions

Also disclosed herein are pharmaceutical compositions that include the conjugate and a pharmaceutically acceptable excipient. Examples of pharmaceutically acceptable excipients include, but are not limited to, buffering agents (e.g., phosphate buffered saline), carbohydrates (e.g., glucose, trehalose, starch, etc.), and combinations thereof. The description of the conjugate, hydrophilic polymer, diABZI, and linker above may be applied to the disclosed pharmaceutical compositions.

### B. Administration

The pharmaceutical composition can be administered prophylactically or therapeutically. In prophylactic administration, the pharmaceutical composition can be administered in an amount sufficient to induce a response. In therapeutic applications, the pharmaceutical composition can be administered to a subject in need thereof in an amount sufficient to elicit a therapeutic effect. Amounts effective for this use will depend on, e.g., the particular composition of the conjugate regimen administered, the manner of administration, the stage and severity of the disease, the general state of health of the patient, and the judgment of the prescribing physician.

The conjugate or pharmaceutical composition thereof can be delivered via a variety of routes to the subject. The conjugate or pharmaceutical composition thereof can be delivered via systemic administration or locally at a site of interest. Example delivery routes include, but are not limited to, intravenous and locally at the site of injury (e.g., site associated with a cancer). In some embodiments, the conjugate or pharmaceutical composition thereof is administered intravenously or locally at a site of interest.

The pharmaceutical composition may be presented in a single dose or as divided doses administered at appropriate intervals, for example, as two, three, four or more sub-doses per day.

The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and subjects treated, the particular conjugates and/or compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

Dosage amount and interval may be adjusted individually to provide plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Pharmaceutical compositions can be administered using a regimen which maintains plasma levels above the MEC for 10-90% of the time, such as between 30-90% or between 50-90%. In cases of local administration or selective uptake, the effective local concentration of the conjugate may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

### C. Methods of Modulating a STING Pathway

Disclosed herein are methods of modulating a STING pathway in a subject (e.g., in need thereof). The method can include administering to the subject an effective amount of the conjugate or a pharmaceutically acceptable salt thereof. The conjugate can be administered optionally with a pharmaceutically acceptable excipient as disclosed herein.

The STING pathway is involved in a number of different diseases and disorders. For example, the subject can have a cancer, a viral infection, or multiple sclerosis. In some embodiments, the subject has cancer. Example cancers include, but are not limited to, melanoma, breast cancer, neuroblastoma, renal cell carcinoma, colon cancer, lung cancer, glioma, glioblastoma, pancreatic cancer, osteosarcoma, ovarian cancer, cervical cancer, bladder cancer, T and B cell lymphomas, medulloblastoma, head and neck cancer (HNSCC), liver cancer, and prostate cancer.

The method of modulating the STING pathway in a subject can lead to advantageous benefits. For example, the method can increase serum levels of a cytokine in the subject. An example cytokine is interferon β (IFN-β). In some embodiments, the method can increase IFN-β serum concentration in the subject within 1 to 4 hours after administration.

### D. Methods of Treating Cancer

Further provided herein are methods of treating cancer in a subject having a cancer. The method can include administering to the subject an effective amount of the conjugate or a pharmaceutically acceptable salt thereof. The conjugate can be administered optionally with a pharmaceutically acceptable excipient as disclosed herein.

The subject can have melanoma, breast cancer, neuroblastoma, renal cell carcinoma, colon cancer, lung cancer, glioma, glioblastoma, pancreatic cancer, osteosarcoma, ovarian cancer, cervical cancer, bladder cancer, T and B cell lymphomas, medulloblastoma, head and neck cancer (HNSCC), liver cancer, or prostate cancer. In some embodiments, the cancer is breast cancer, neuroblastoma, melanoma, renal cell carcinoma, glioblastoma, or colon cancer.

The disclosed conjugates can have improved therapeutic efficacy compared to diABZI administered alone to the subject. This improvement of the conjugate can be increased when the conjugate is administered systemically. Accordingly, in some embodiments, the conjugate is administered systemically.

The description of the conjugate, pharmaceutical composition, hydrophilic polymer, diABZI, and linker above may be applied to the disclosed methods of modulating a STING pathway and treating cancer.

### 4. MODIFIED DIABZI COMPOUNDS

In another aspect, disclosed are modified diABZI compounds. The modified diABZI compounds can be attached to the hydrophilic polymer through the linker as disclosed herein.

The modified diABZI compound can be a compound of formula (VI), or a pharmaceutically acceptable salt thereof,

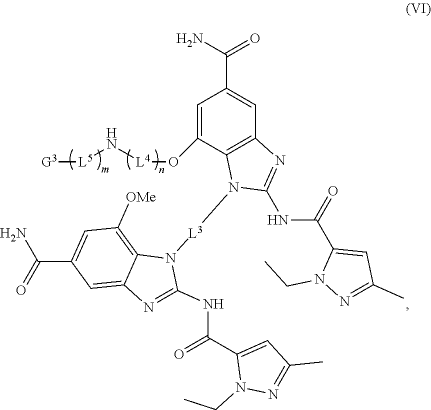

wherein: L³ is C₃₋₈alkylene or C₃₋₈alkenylene; L⁴ is C₁₋₆alkylene or C₁₋₆alkenylene; L⁵ is

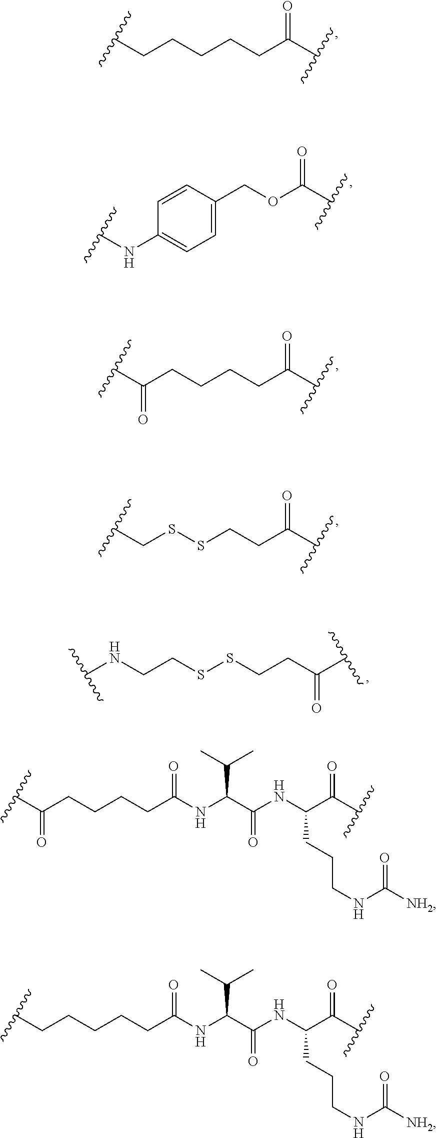

or a combination thereof G³ is

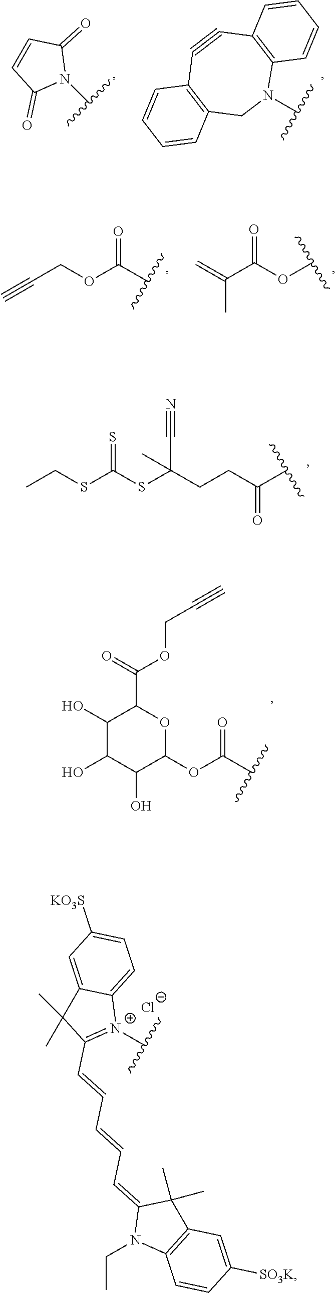

or a combination thereof; m is 0-4; and n is 1-2.

In some embodiments, L³ is a C₃₋₈alkenylene. In some embodiments, L³ is a C₃₋₆alkenylene.

In some embodiments, L⁴ is a C₁₋₆alkylene. In some embodiments, L⁴ is a C₁₋₄ alkylene.

In some embodiments, n is 1.

In some embodiments, m is 1-3. In some embodiments, m is 0, 1, 2, or 3.

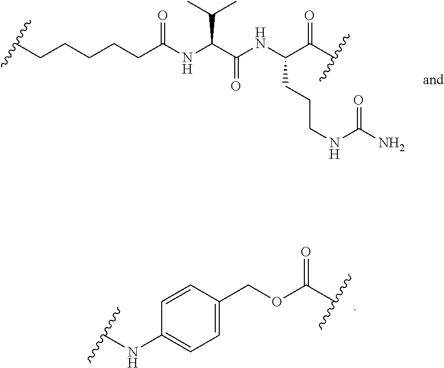 [SMILES: *CCCCCC(=O)N[C@H](C(=O)N[C@@H](CCCNC(N)=O)C(*)=O)C(C)C ; *Nc1ccc(COC(*)=O)cc1]

In some embodiments, L⁵ is

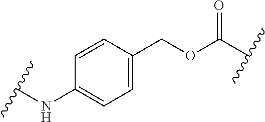 [SMILES: *Nc1ccc(COC(*)=O)cc1]

and m is 1, 2, or 3

In some embodiments, L^{S} is H and m is 1, 2, or 3.

In some embodiments, compounds of formula (VI) can be used as RAFT monomers and/or chain transfer agents. For example, G³ can be

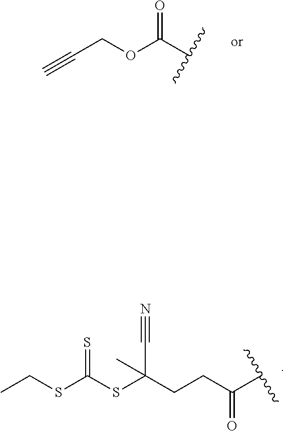

In some embodiments, L⁵ is present. In other embodiments, L⁵ is absent.

In some embodiments, the compound is of formula (VI-a):

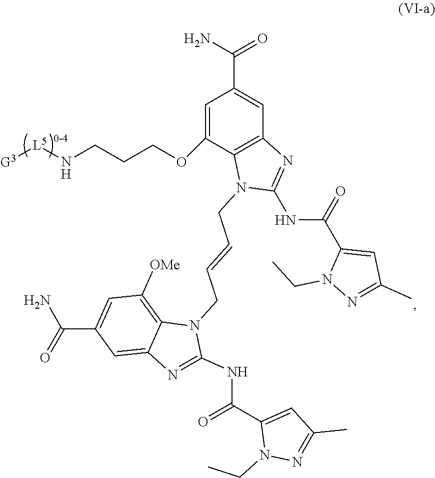

wherein G³ and L⁵ are defined as described for formula (VI).

In some embodiments, the compound is of formula (VI-b):

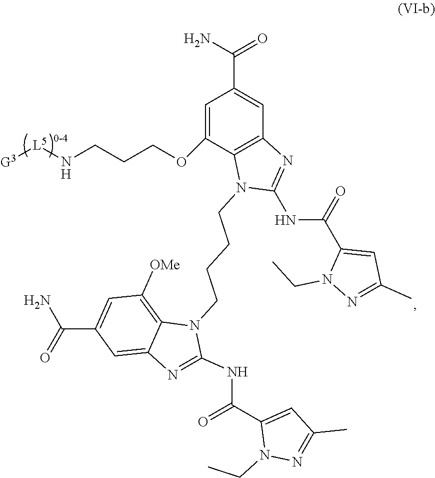

wherein G³ and L⁵ are defined as described for formula (VI).

In some embodiments, the compound is selected from the group consisting of:

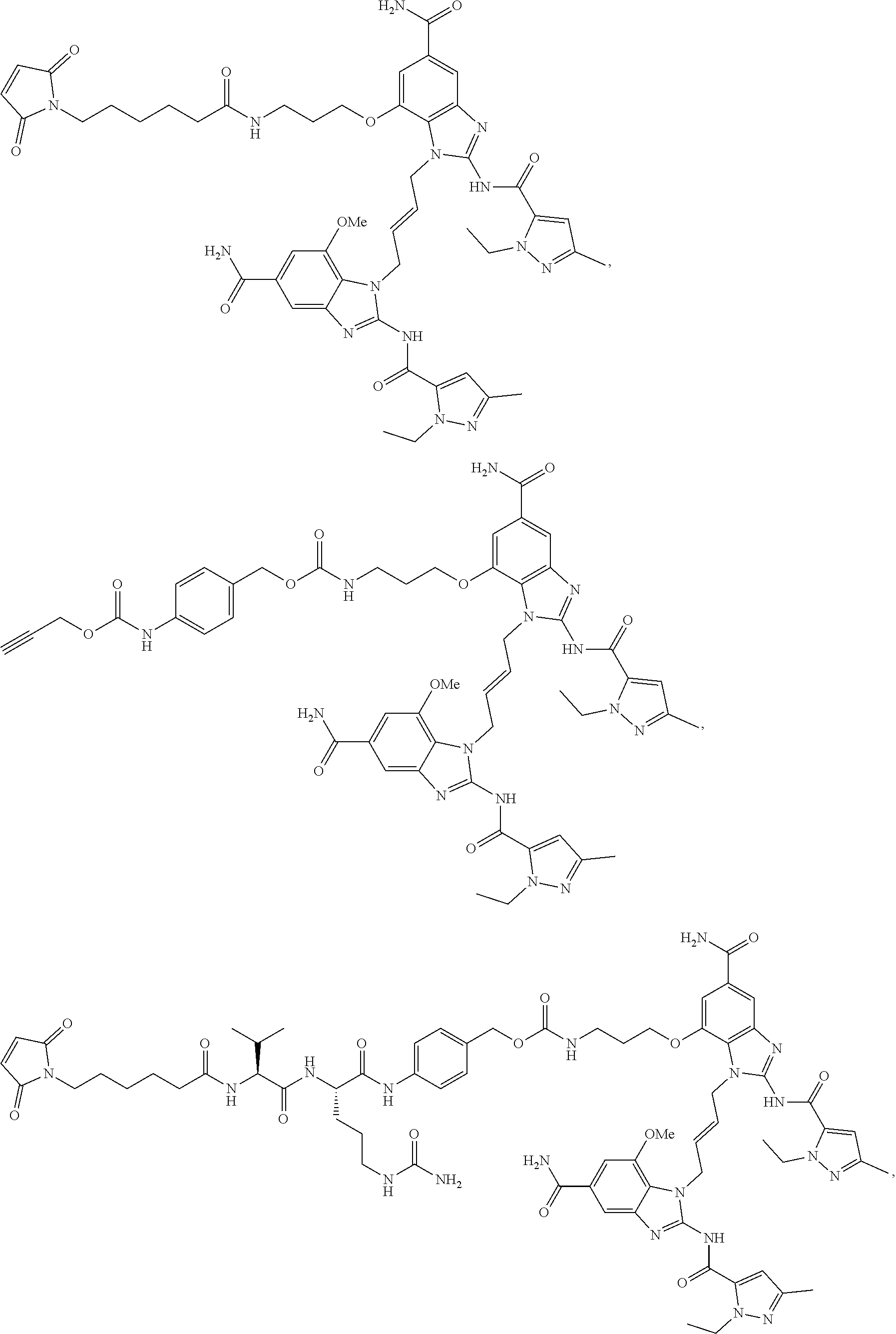

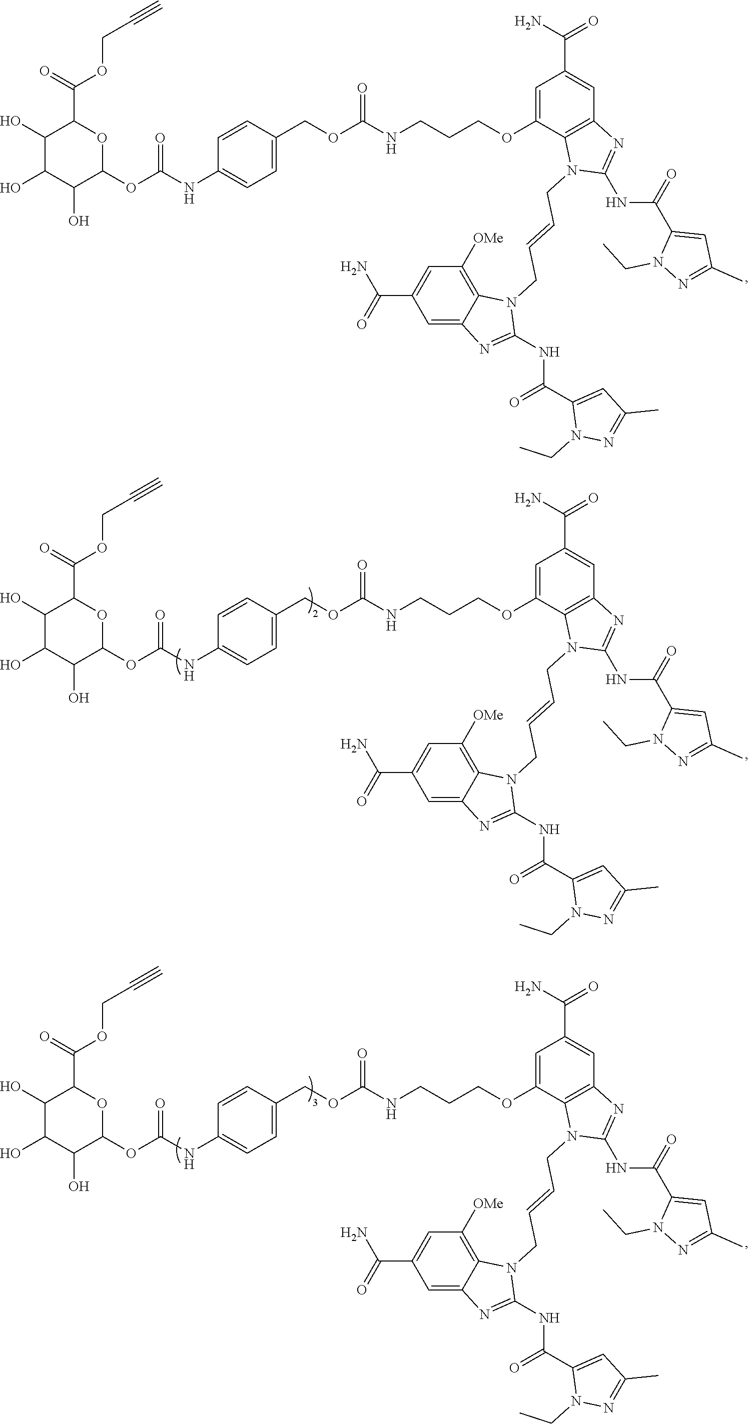

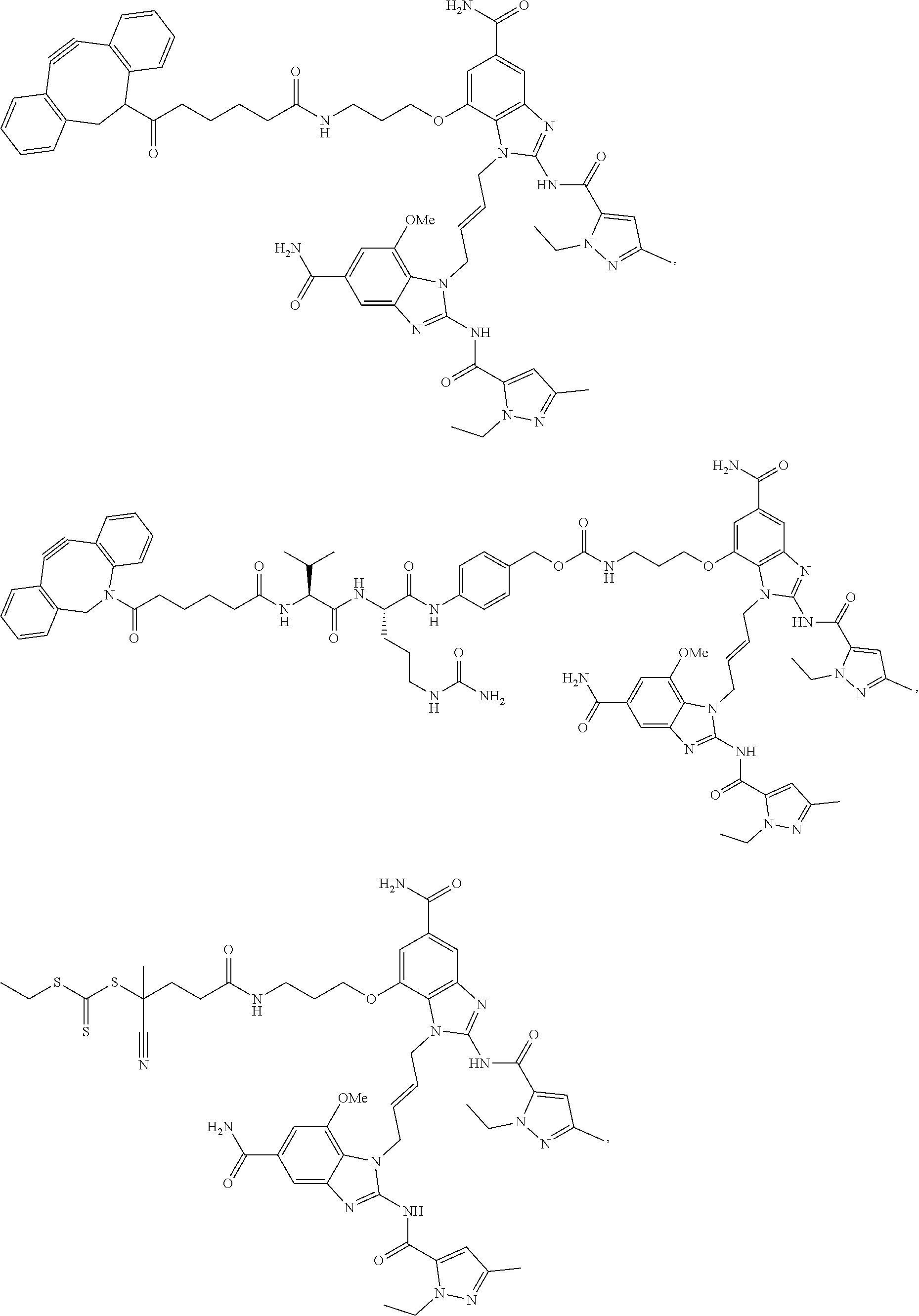

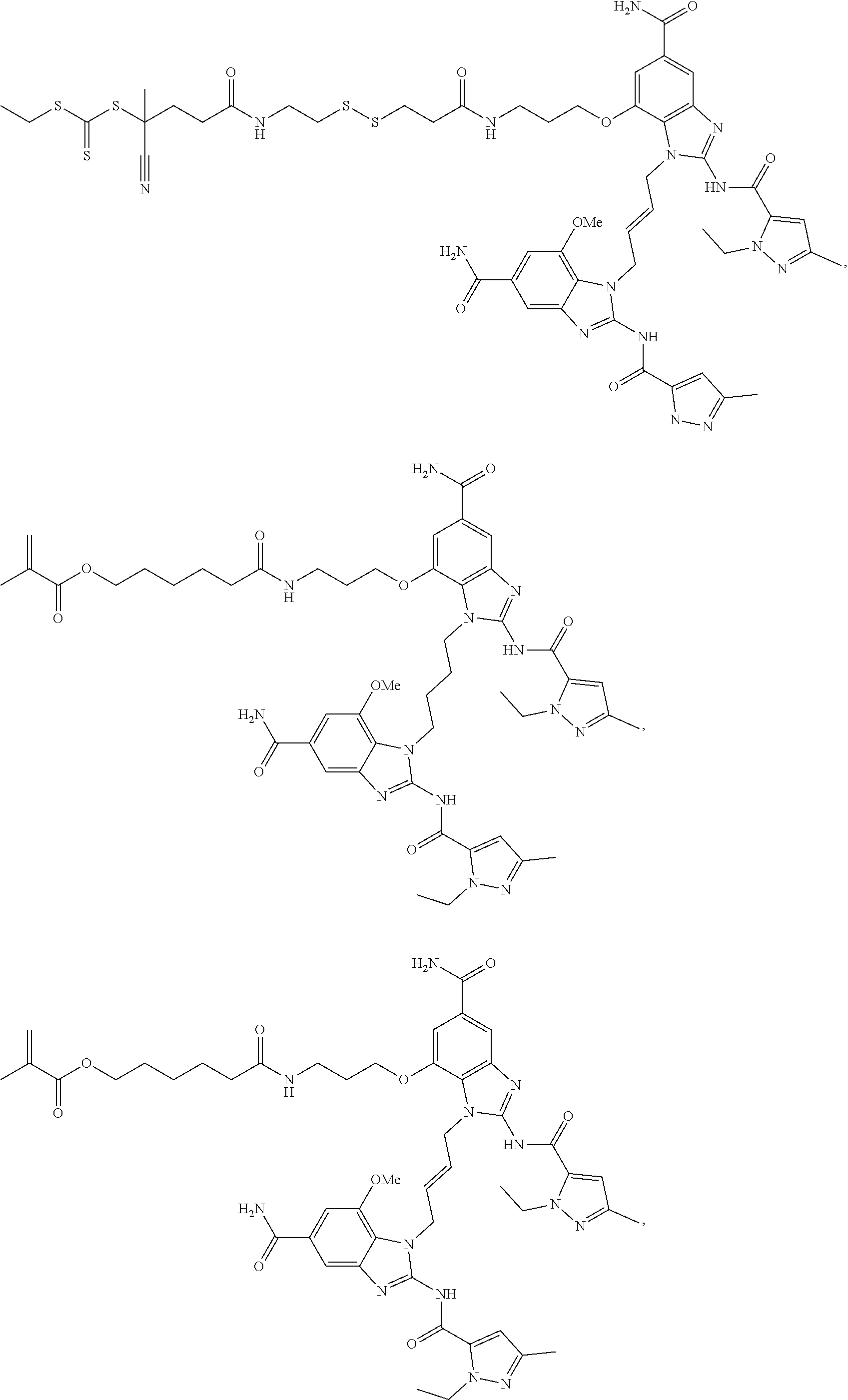

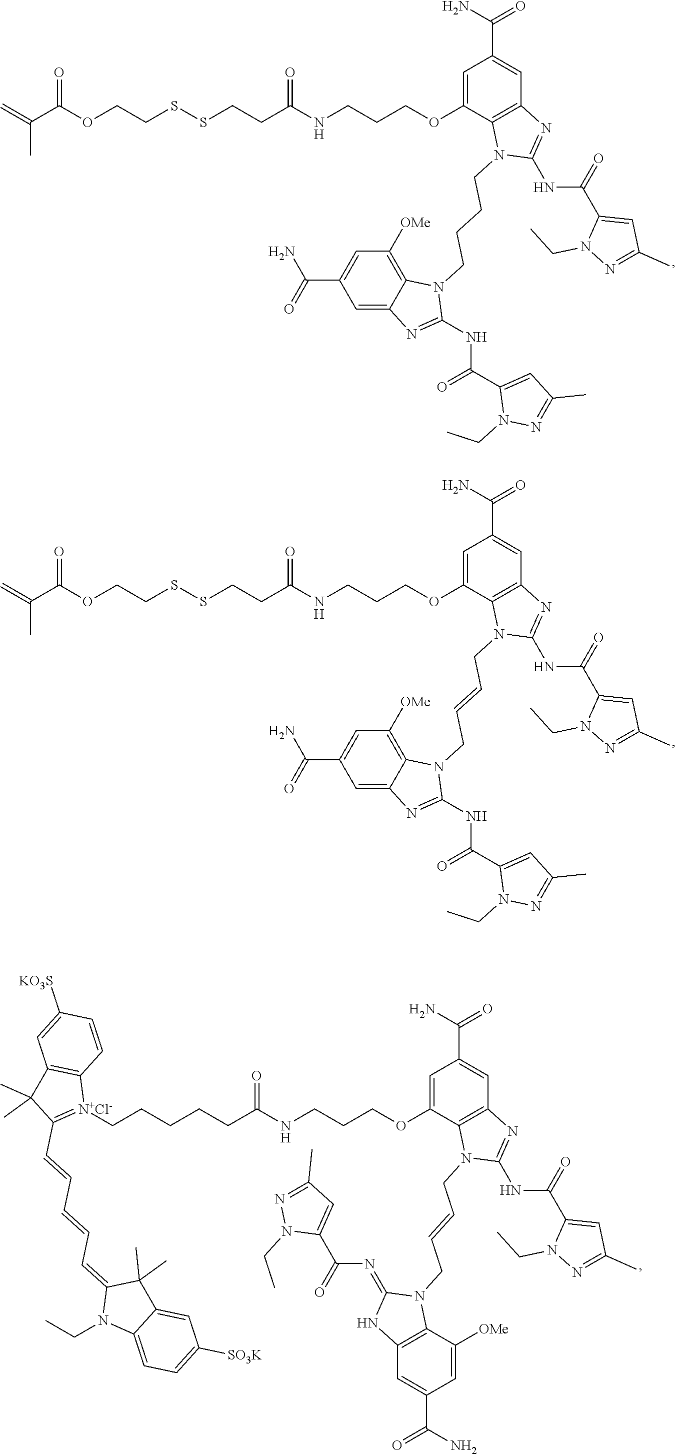

and a pharmaceutically acceptable salt thereof.

The compounds of formula (VI) may also be prepared as pharmaceutical compositions and salts as described herein, as well as be used in methods as described herein.

The disclosed invention has multiple aspects, illustrated by the following non-limiting examples.

### 5. EXAMPLES

### Example 1

Synthesis of Diamidobenzimidazole (diABZI)

### 1. Synthesis of Compounds

### Abbreviations

DIPEA is N,N-Diisopropylethylamine;
Hunig's base is DIPEA;
BuOH is n-butyl alcohol;
Na₂S₂O₄ is sodium dithionite;
MeOH is methanol;
EtOAc is ethyl acetate;
DCM is dichloromethane;
DMF is dimethylformamide;
EDC is 1-ethyl-3-(3-dimethylaminopropyl)carbodiimide);
TEA is triethylamine;
TFA is trifluoroacetic acid;
DMSO is dimethyl sulfoxide;
Rf is retention factor;
TLC is thin-layer chromatography;
HRMS is high-resolution mass spectroscopy;
NMR is nuclear magnetic resonance;
atm is atmospheric pressure;
eq. or equiv. is equivalents;
rt or RT is room temperature;
hr or h is hour; and
min or mins is minutes.

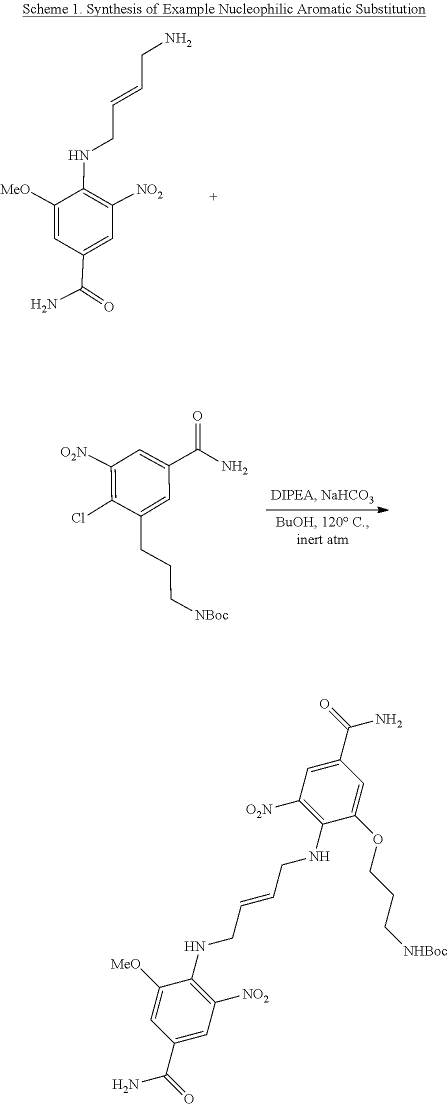

In a sealable tube, a mixture of tert-butyl (3-(5-carbamoyl-2-chloro-3-nitrophenoxy)propyl)carbamate (14.2 g, 38.0 mmol, 1.1 eq), (E)-4-((4-aminobut-2-en-1-yl)amino)-3-methoxy-5-nitrobenzamide, hydrochloride (11.0 g, 34.6 mmol, 1 eq), Hunig's base (30 mL, 172.8 mmol, 5 eq), and n-butanol (140 mL) was maintained at rt and argon was bubbled through the solution for 5 min. The mixture was sealed under an inert atmosphere, and heated to 120° C. for 24 h, during which time the reaction mixture become homogeneous solution with brick red color. After cooling to rt the solid product was isolated by filtration washed with 25 mL ethanol followed by washing with 150 mL ether and dried to afford the desired product (16.5 g, 26.7 mmol, 77%) as a brick red solid.

(E)-(3-(5-carbamoyl-2-((4-((4-carbamoyl-2-methoxy-6-nitrophenyl)amino)but-2-en-1-yl)amino)-3-nitrophenoxy)propyl)carbamate ¹H NMR (400 MHz, DMSO) δ 8.15 (s, 2H), 8.00 (broad s, 2H), 7.72-7.68 (m, 2H), 7.51 (s, 2H), 7.31 (broad s, 2H), 6.91 (t, J=5.0 Hz, 1H), 5.68-5.58 (m, 2H), 4.12-4.04 (m, 4H), 4.00 (t, J=6.2 Hz, 2H), 3.82 (s, 3H), 3.08 (dt, J=6.2, 5.0 Hz, 2H), 1.85 (p, J=6.2 Hz, 2H), 1.35 (s, 9H). HRMS (ESMS) Calculated for C₂₇H₃₅N₇O₁₀ [M+H]⁺: 618.2523, found 618.2507.

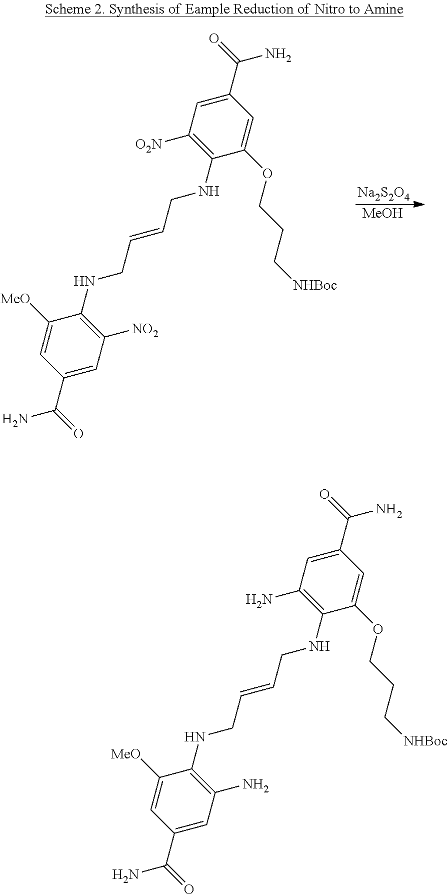 [SMILES: "MeOH" ; COc1cc(C(N)=O)cc([N+](=O)[O-])c1NC/C=C/CNc1c(OCCCNC(=O)OC(C)(C)C)cc(C(N)=O)cc1[N+](=O)[O-] ; "Na2S2O4" ; COc1cc(C(N)=O)cc(N)c1NC/C=C/CNc1c(N)cc(C(N)=O)cc1OCCCNC(=O)OC(C)(C)C]

A solution of tert-butyl (E)-(3-(5-carbamoyl-2-((4-((4-carbamoyl-2-methoxy-6-nitrophenyl)amino)but-2-en-1-yl)amino)-3-nitrophenoxy)propyl)carbamate (16.5 g, 26.7 mmol, 1 eq) in 300 mL methanol was maintained at rt. To which aqueous solution of sodium dithionate (65 g, 374 mmol, 14 eq) in 150 mL water added. After 5 min ammonium hydroxide (87 mL, 668 mmol, 25 eq) added to the reaction mixture. The reaction mixture was allowed to stir for 45 min during which time the color of the reaction mixture changed from orange to light yellow. After consumption of starting material, as judged by TLC analysis, the reaction mixture was filtered through celite. NaCl added to saturate the aqueous layer which was extracted by EtOAc (3×200 mL). The organic layer was dried over Na₂S₂O₄ and evaporated in vacuo to obtain the crude product. The crude reaction mixture was purified by column chromatography on basic alumina (5% to 35% MeOH:DCM) to get the desired product as a thick yellow oil (6.7 g, 12 mmol, 45%). (R_{f}=0.4 in 10% MeOH in DCM on silica gel TLC).

tert-butyl(E)-(3-(3-amino-2-((4-((2-amino-4-carbamoyl-6-methoxyphenyl)amino)but-2-en-1-yl)amino)-5-carbamoylphenoxy)propyl)carbamate 1H NMR (400 MHz, DMSO) δ 7.60 (broad s, 2H), 6.96 (broad s, 2H), 6.89 (t, J=5.1 Hz), 6.85 (dd, J=4.0, 1.7 Hz, 2H), 6.77 (merged dd, 2H), 5.72-5.63 (m, 2H), 4.64 (d, J=4 Hz, 4H), 3.94 (t, J=6.1 Hz, 2H), 3.82-3.75 (m, 2H), 3.74 (s, 3H), 3.55-3.45 (m, 4H), 3.10 (dt, J=6.1, 5.1 Hz, 2H), 1.83 (p, J=6.2 Hz, 2H), 1.36 (s, 9H). HRMS (ESMS) Calculated for C₂₇H₃₉N₇O₆ [M+H]+: 558.3040, found 558.3023.

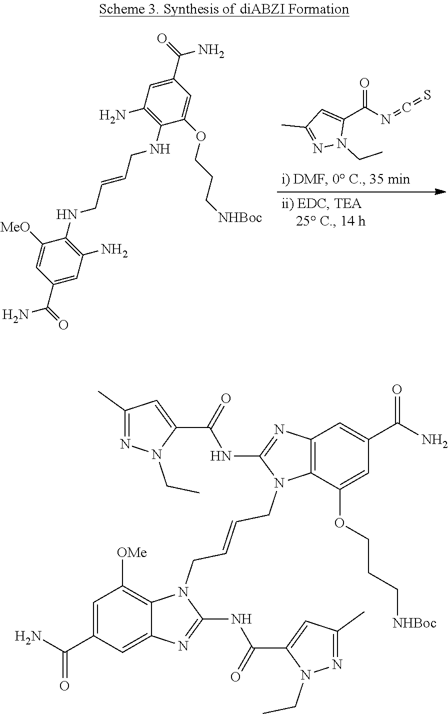 [SMILES: COc1cc(C(N)=O)cc(N)c1NC/C=C/CNc1c(N)cc(C(N)=O)cc1OCCCNC(=O)OC(C)(C)C ; CCn1nc(C)cc1C(=O)N=C=S ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCNC(=O)OC(C)(C)C)c21]

To a stirred solution of tert-butyl (E)-(3-(3-amino-2-((4-((2-amino-4-carbamoyl-6-methoxyphenyl)amino) but-2-en-1-yl)amino)-5-carbamoylphenoxy)propyl)carbamate (6.7 g, 12.0 mmol, 1 eq) in DMF (60 mL) add solution of 1-ethyl-3-methyl-1H-pyrazole-5-carbonyl isothiocyanate (5.2 g, 26.4 mmol, 2.2 eq) in DMF (12 mL) under inert atmosphere and stir for 45 min. EDC (5.8 g, 30.0 mmol, 2.5 eq) followed by triethylamine (8.4 mL, 60.0 mmol, 5 eq) were added to the reaction mixture and stirred overnight. The reaction mixture was diluted with diethyl ether to precipitate out the crude reaction mixture. Solid was filtered and resuspended in aqueous ammonium chloride solution (10 g NH₄Cl in 100 mL water) stirred for 15 min and filtered followed by washing with water (2×50 mL), diethyl ether (2×50 mL) to obtain the desired intermediate (8.1 g, 9.22 mmol, 77%) as an off white solid.

tert-butyl(E)-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)carbamate ¹H NMR (400 MHz, DMSO) δ 7.95 (broad s, 2H), 7.95 (broad s, 2H), 7.63 (d, J=2.63 Hz, 2H), 7.33 (broad s, 2H), 7.30 (d, J=4.46 Hz, 2H), 6.85 (t, J=5.1 Hz, 1H), 6.5 (s, 2H), 5.89-5.77 (m, 2H), 4.95-4.89 (m, 4H), 4.51 (q, J=6.89 Hz, 4H), 3.99 (t, J=6.1 Hz, 2H), 3.72 (s, 3H), 3.61 (dt, J=6.1, 5.1 Hz, 2H), 2.09 (s, 6H), 1.70 (p, J=6.1 Hz, 2H), 1.32 (s, 9H), 1.26 (t, J=6.89 Hz, 6H). HRMS (ESMS) Calculated for C₄₃H₅₃N₁₃O₈[M+H]⁺: 880.4218, found 880.4174.

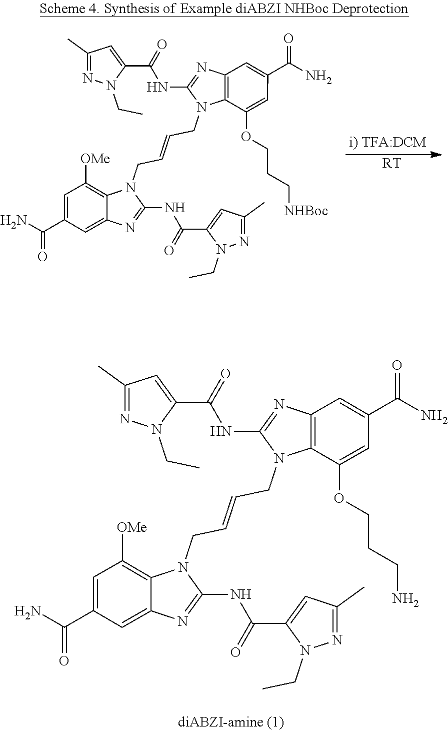 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCNC(=O)OC(C)(C)C)c21 ; "RT" ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN)c21]

To a stirred suspension of tert-butyl (E)-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)carbamate (6.6 g, 7.5 mmol, 1 eq) in dichloromethane (70 mL) under inert atmosphere at room temperature add trifluoroacetic acid (5.7 mL, 75 mmol, 10 eq) was added dropwise, which converted the suspension into a solution and stirred for 6 hr. The diethyl ether added to precipitate out the solid product as tri TFA salt. The solid was filtered over Buchner funnel and washed with diethyl ether (2×100 mL) to obtain the tri TFA salt of desired compound (1) as an off-white solid (6.5 g, 5.8 mmol, 77%).

(E)-7-(3-aminopropoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (1) ¹H NMR (400 MHz, DMSO) δ 7.96 (broad s, 2H), 7.71 (broad s, 2H), 7.64 (d, J=5.4 Hz, 2H), 7.36 (broad s, 2H), 7.32 (dd, J=9.5, 0.9 Hz, 2H), 6.5 (d, J=9.0 Hz, 2H), 5.84-5.73 (m, 2H), 4.92-4.89 (m, 4H), 4.54-4.47 (m, 4H), 4.09 (t, J=6.1 Hz, 2H), 3.71 (s, 3H), 2.92-2.84 (m, 2H), 2.11 (s, 3H), 2.09 (s, 3H), 1.89 (p, J=6.1 Hz, 2H), 1.25 (dt, J=7.08, 4.6 Hz, 6H). ¹³C NMR (151 MHz, DMSO) δ 168.1, 158.9, 158.7, 158.5, 158.3, 145.6, 145.3, 144.4, 130.5, 130.5, 128.4, 128.2, 109.7, 109.6, 106.2, 105.6, 65.9, 56.4, 46.1, 45.1, 36.6, 27.1, 16.6, 13.6. HRMS (ESMS) Calculated for C₃₈H₄₅N₁₃O₆ [M+H]⁺: 780.3694, found 780.3663.

### Example 2

Synthesis and Functional Activity of diABZI with Maleimide Functionalized Linkers

### 1. Synthesis of Compounds

### Abbreviations

Et₃N is triethylamine;
MeOH is methanol;
DCM is dichloromethane;
DMSO is dimethyl sulfoxide;
DMF is dimethylformamide
HRMS is high-resolution mas spectroscopy;
NMR is nuclear magnetic resonance;
atm is atmospheric pressure;
eq. or equiv. is equivalents;
rt or RT is room temperature;
hr or h is hour; and
min is minutes

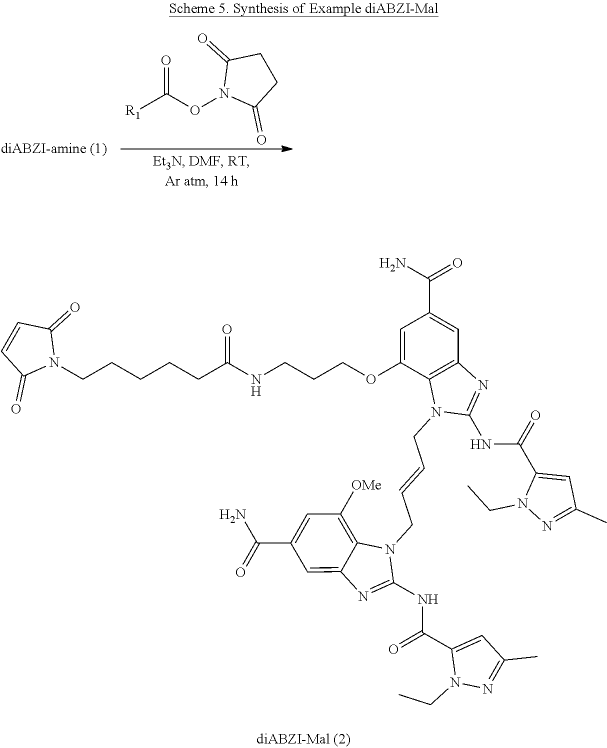

To a stirred solution of (E)-7-(3-aminopropoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide tris trifluoracetic acid (1) (200 mg, 0.18 mmol, 1 eq) and Hunig's base (0.12 mL, 0.89 mmol, 5 eq) in DMF (3 mL) dropwise added solution 2,5-dioxopyrrolidin-1-yl 6-(2,5-dioxo-2,5-dihydro-1H-pyrrol-1-yl)hexanoate (66 mg, 0.2 mmol, 1.2 eq) in DMF (2 mL) under inert atmosphere and stirred overnight at room temperature. The diethyl ether added to precipitate out the crude solid desired product. The crude solid was purified over silica gel chromatography (DCM:MeOH 0-20%) to obtain the desired product diABZI-Mal (2) as an off-white solid (80 mg, 0.08 mmol, 46%).

diABZI Mal (2) ¹H NMR (400 MHz, DMSO) δ 7.95 (broad s, 2H), 7.76 (t, J=5.5 Hz, 1H), 7.63 (s, 2H), 7.32 (broad s, 2H), 7.29 (d, J=6.2 Hz, 2H), 6.97 (s, 2H), 6.5 (d, J=7.6 Hz, 2H), 5.89-5.77 (m, 2H), 4.95-4.88 (m, 4H), 4.54-4.48 (m, 4H), 3.96 (t, J=6.1 Hz, 2H), 3.71 (s, 3H), 3.09 (dt, J=6.2, 5.9 Hz, 2H), 2.10 (s, 3H), 2.09 (s, 3H), 1.98 (t, J=7.2 Hz, 2H), 1.67 (p, J=6.1 Hz, 2H), 1.47-1.40 (m, 4H), 1.25 (dt, J=7.1, 3.2 Hz, 6H), 1.18-1.10 (m, 2H). ¹³C NMR (151 MHz, DMSO) δ 172.4, 171.5, 168.1, 167.3, 152.5, 145.5, 145.3, 144.7, 140.4, 140.3, 134.9, 130.5, 130.5, 128.7, 128.2, 120.1, 109.7, 56.4, 46.0, 37.4, 35.6, 29.2, 28.2, 26.3, 25.2, 16.6, 13.6. HRMS (ESMS) Calculated for C₄₈H₅₆N₁₄O₉[M+H]⁺: 973.4433, found 973.4417.

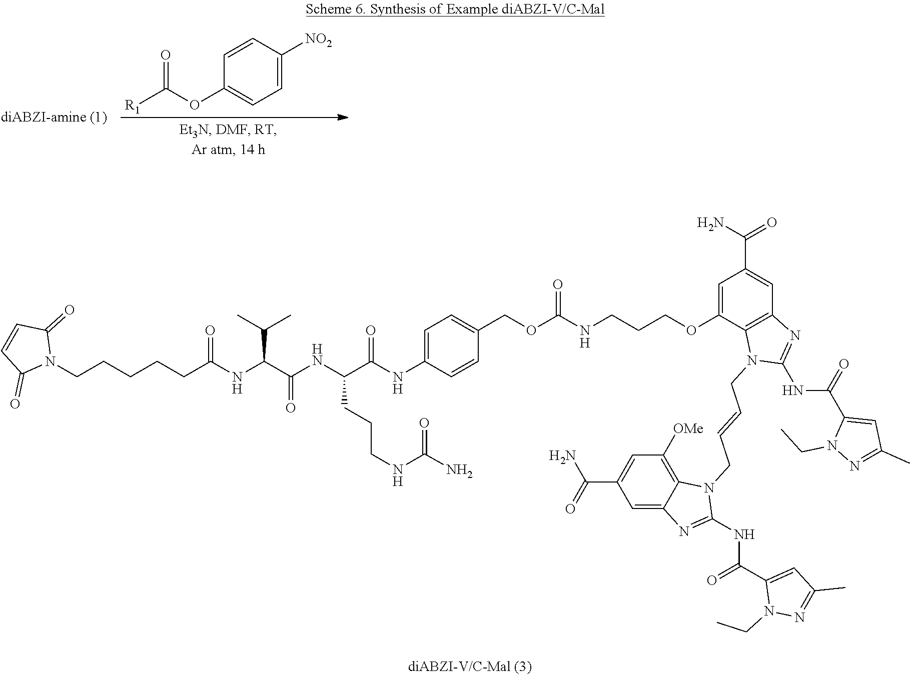

To a stirred solution of (E)-7-(3-aminopropoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide tris trifluoracetic acid (1) (350 mg, 0.3 mmol, 1 eq) and hunig's base (0.27 mL, 1.56 mmol, 5 eq) in DMF (5 mL) dropwise added solution of 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (253 mg, 0.34 mmol, 1.1 eq) in DMF (3 mL) under inert atmosphere which turned reaction mixture into a bright yellow color that indicate releasing of para nitrophenol. The reaction mixture stirred overnight at room temperature. The diethyl ether added to precipitate out the crude solid desired product. The solid was filtered over Büchner funnel and resuspended in 1:1 (DCM:MeOH) 5 mL and stirred for 2 hr. The solid was filtered over Buchner funnel to obtain the pure desired product diABZI-3 as a light pink solid (413 mg, 0.29 mmol, 96%).

diABZI V/C Mal (3) ¹H NMR (400 MHz, DMSO) δ 9.95 (s, 1H), 8.06 (d, J=7.5 Hz, 1H), 7.95 (broad s, 2H), 7.79 (d, J=8.5 Hz, 1H), 7.63 (d, J=6.7 Hz, 2H), 7.56 (d, J=8.5 Hz, 2H), 7.34-7.23 (m, 7H), 6.99 (s, 2H), 6.5 (d, J=5.6 Hz, 2H), 5.96 (t, J=5.0 Hz, 1H), 5.88-5.79 (m, 2H), 5.40 (s, 2H), 4.93-4.87 (m, 6H), 4.53-4.49 (m, 4H), 4.38-4.35 (m, 1H), 4.19-4.16 (m, 1H), 3.98 (t, J=5.0 Hz, 2H), 3.71 (s, 3H), 3.40-3.34 (m, 2H), 3.08-2.90 (m, 4H), 2.20-1.92 (m, 9H), 1.73-1.54 (m, 4H), 1.52-1.14 (m, 16H), 0.84 (d, J=6.7 Hz, 3H), 0.80 (d, J=6.7 Hz, 3H). ¹³C NMR (151 MHz, DMSO) δ 172.7, 171.8, 171.5, 171.0, 168.1, 167.3, 159.4, 156.6, 145.5, 145.3, 140.4, 139.0, 134.9, 132.2, 130.5, 129.1, 119.4, 109.7, 65.5, 58.0, 56.4, 53.6, 46.0, 37.5, 35.4, 30.8, 29.8, 28.2, 27.3, 26.2, 25.4, 19.7, 18.7, 16.6, 13.6. HRMS (ESMS) Calculated for C₆₇H₈₃N₁₉O₁₄ [M+H]⁺: 1378.6445, found 1378.6415.

### 2. Biological Evaluation of Compounds

Using diABZI-amine as a starting material, maleimide groups were integrated for conjugation to thiol groups on polymeric carriers via thiol-maleimide Michael addition reactions. The primary amine of diABZI-amine (1) was reacted with 6-Maleimidohexanoic acid N-hydroxysuccinimide ester in which the primary amine attacked the carbonyl of the activated NHS ester for nucleophilic addition-elimination reaction to form the desired maleimide-functionalized analog, diABZI-Mal (2) (Scheme 5), for conjugation to thiol-containing carriers via a non-cleavable five methylene (—CH₂—) spacer. To allow for diABZI release from carriers, a variant was synthesized with a cathepsin-cleavable valine-citrulline-PAB linker, which allows for regeneration of diABZI-amine (1) within the endo-lysosome and/or tumor microenvironment where levels of capthesin-B are often elevated. In order to synthesize cathepsin-B cleavable analog, the primary amine of diABZI-amine (1) was treated with the carbonyl group of the activated PNP ester in which the primary amine attacked the carbonyl group for nucleophilic addition-elimination reaction to form the desired analog with cathepsin-cleavable valine-citrulline-PAB linker with terminal maleimide group for bioconjugation, which is denoted as diABZI-V/C-Mal (3) (Scheme 6). The structure of diABZI-Mal (2) and diABZI-V/C-Mal (3) were deduced from its spectral features and confirmed by ¹H NMR, ¹³C NMR and mass spectrometry. The distinctive peak at 6.9 PPM integrating for two protons in ¹H NMR corresponds to methine protons of maleimide group for both compounds (2) and (3), this peak can be used to track thiol-maleimide Michael addition with diverse drug delivery carriers. In summary, maleimide-functionalized diABZI variants were synthesized for covalent conjugation to thiol-containing carriers via a non-cleavable 5 carbon spacer diABZI-Mal (2) or with a cathepsin-cleavable linker diABZI-V/C-Mal (3).

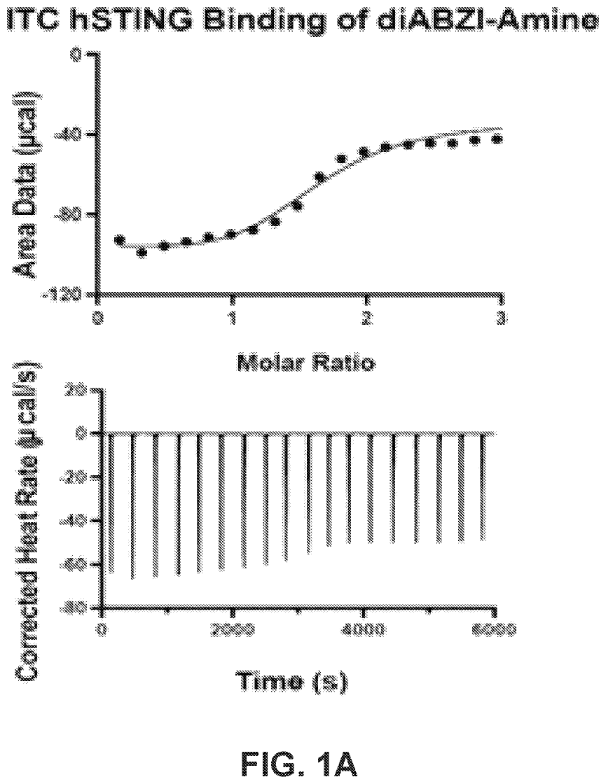

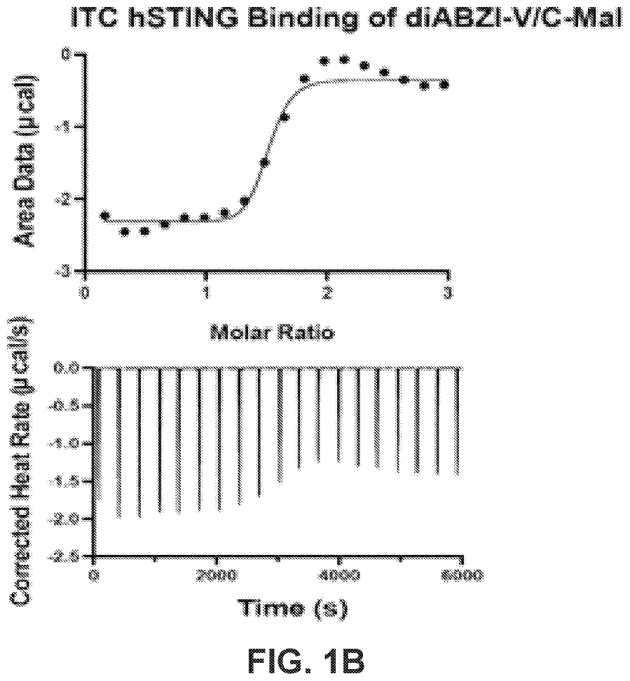

Next, the impact of modification on STING binding was compared to control STING agonists using isothermal calorimetry with recombinant human STING (hSTING; STING isoform 1) to estimate binding affinity (FIG. 1A,1B). All three diABZI analogs (compounds (1), (2), and (3)) exhibited binding to hSTING with approximately the same K_{d} as a previously described diABZI molecule (Table 1).

TABLE 1
Binding (K_{D}) of diABZI, diABZI-amine (1),
diABZI-Mal (2), and diABZI-V/C-Mal (3).

| Agonist | K_{D} (nM) |
|---|---|
| 2′,3′ cGAMP | ~4.00 |
| diABZI | 527 ± 321 |
| diABZI-amine | 70 ± 62 |
| diABZI-Mal | 890 ± 547 |
| diABZI-V/C-Mal | 207 ± 150 |

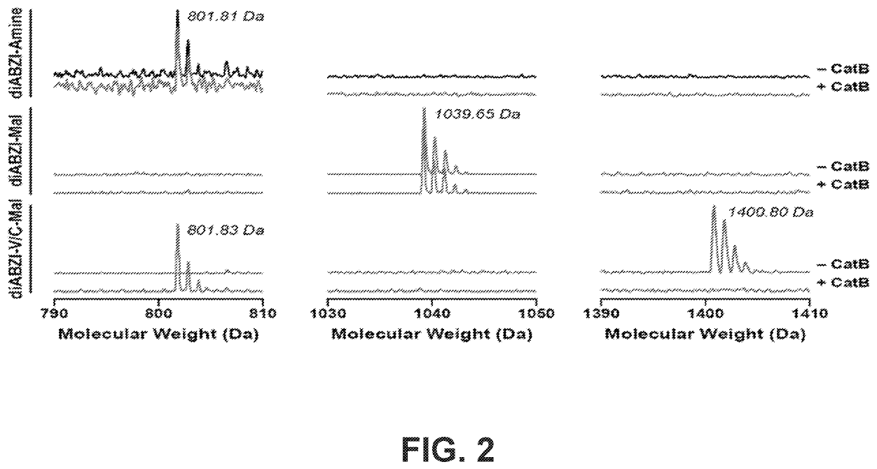

The enzyme cleavability of diABZI-V/C-Mal (3) was evaluated via incubation with cathepsin-B for followed by MALDI-MS to demonstrate accessibility of the Val-Cit-PAB linker by cathepsin B and regeneration of diABZI-amine (1) upon enzyme cleavage (FIG. 2). Incubation of 50 μM diABZI-V/C-Mal (3) with 0.2 μM cathepsin B resulted in a shift in molecular weight from 1400.8 Da to 801.83 Da, consistent with the molecular weight of diABZI-amine (1) sodium adduct, whereas no change in molecular weight was observed under the same conditions for diABZI-Mal (2), an analog that lacks an enzyme-cleavable spacer.

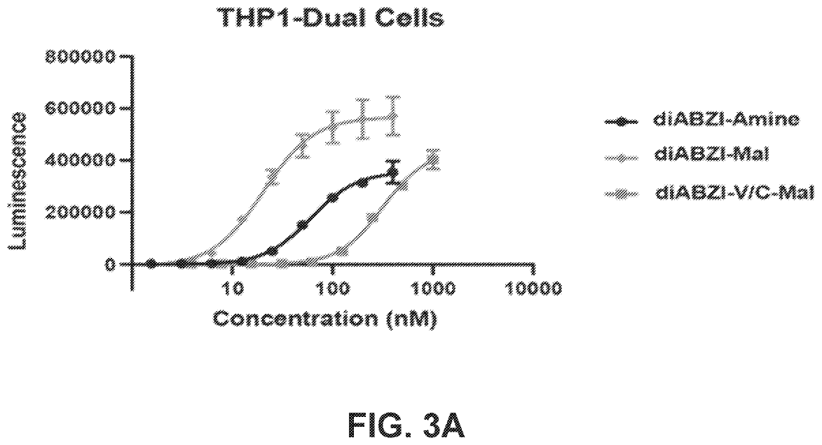

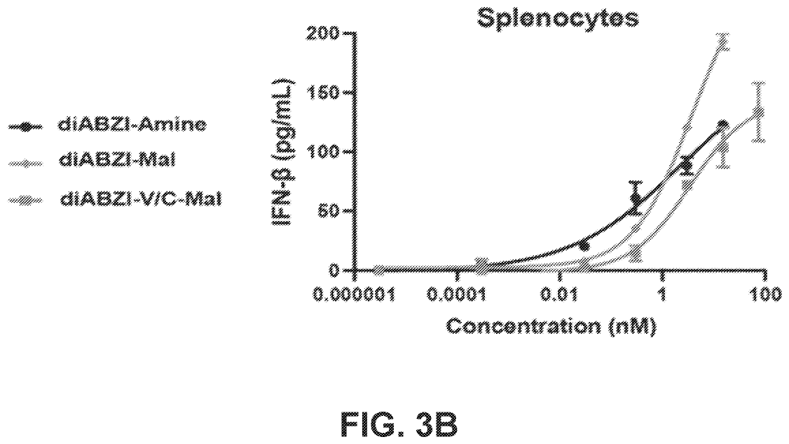

The immunostimulatory activity of the three diABZI analogs in vitro were evaluated. Initially, the activity in human monocyte-derived THP1-Dual™ reporter cells that are engineered to secrete luciferase upon activation of interferon regulatory factor (IRF) signaling was tested Dose-response curves indicated that relatively similar potency between diABZI-amine (1), diABZI-Mal (2), and diABZI-V/C-Mal (3) with estimated EC50 values of 60.9 nM, 20.5 nM, and 314 nM, respectively (FIG. 3A). The differences in EC50 values observed between the compounds may be attributed to differences in cell membrane permeability since similar STING binding affinity was observed between the analogs. The activity in primary murine splenocytes, which comprises a mixture of different immune cell populations, using ELISA was used to quantify secreted interferon-β (IFN-β), a signature cytokine of STING pathway activity. All three compounds stimulated IFN-βproduction to a similar extent, with EC50 values for diABZI-amine (1), diABZI-Mal (2), and diABZI-V/C-Mal (3) of approximately 2.24 nM, 3.62 nM, and 3.38 nM, respectively. (FIG. 3B).

### Example 3

Synthesis and Functional Activity of diABZI with Alkyne Functionalized Linkers

### 1. Synthesis of Compounds

### Abbreviations

DPPA is diphenylphosphoryl azide;
Et₃N is triethylamine;
THF is tetrahydrofuran;
EtOAc is ethyl acetate;
DCM is dichloromethane;
DMF is dimethylformamide;
HRMS is high-resolution mas spectroscopy;
NMR is nuclear magnetic resonance;
TLC is thin-layer chromatography;
Rf is retention factor;
atm is atmospheric pressure;
eq. or equiv. is equivalents;
rt or RT is room temperature;
hr or h is hour; and
min is minutes.

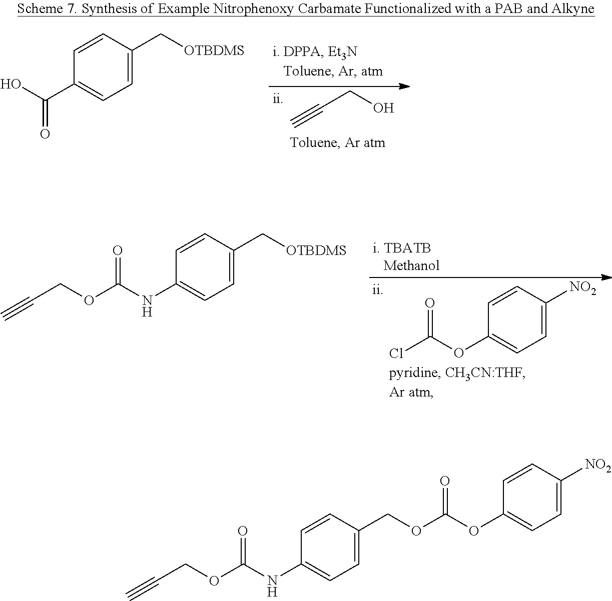

4-(((tert-butyldimethylsilyl)oxy)methyl)benzoic acid (6 g, 22.5 mmol, 1 eq), DPPA (5.7 mL, 26.5 mmol, 1.18 eq) and triethylamine (3.7 mL, 26.5 mmol, 1.18 eq) were dissolved in 90 mL dry toluene under argon atmosphere. The reaction mixture was heated at 85° C. and stirred for 3 h. Afterwards, solution of propargyl alcohol (1 g, 18.0 mmol, 0.8 eq) in 20 mL dry toluene added to the reaction mixture at room temperature and stirred overnight. The solvent was evaporated in vacuo to obtain the crude product. The crude reaction mixture was purified by column chromatography on silica gel (5% to 10% EtOAc:Hexane) to get the desired intermediate prop-2-yn-1-yl (4-(((tert-butyldimethylsilyl)oxy)methyl)phenyl)carbamate (4.2 g, 13.1 mmol, 73%). (R_{f}=0.5 in 10% EtOAc in Hexane on silica gel TLC).

Prop-2-yn-1-yl (4-(((tert-butyldimethylsilyl)oxy)methyl)phenyl)carbamate (1 g, 3.1 mmol, 1 eq) dissolved in 10 mL methanol and stirred at room temperature. To a stirred solution Tetrabutylammonium tribromide (TBATB) (0.15 g, 0.3 mmol, 0.1 eq) added and stirred for 3 h. After consumption of the starting material, as judged by the TLC analyses, the solvent was evaporated in vacuo, and crude product was redissolved in ethyl acetate (50 mL). The organic layer was washed with 0.5M KHSO₄ (2×50 mL) dried over sodium sulphate and evaporated in vacuo to obtain crude product. The crude product was purified over silica gel chromatography (EtOAc:Hexane 10-50%) to obtain the desired alcohol (400 mg, 1.95 mmol, 62%). To a stirred solution of prop-2-yn-1-yl (4-(hydroxymethyl)phenyl)carbamate (400 mg, 1.95 mmol, 1 eq) in ACN:THF (15 mL:2 mL) was added pyridine (200 mg, 2.53 mmol, 1.3 eq) and 4-nitrophenyl carbonochloridate (472 mg, 2.34 mmol, 1.2 eq) under argon atmosphere at 0° C. and stirred for 2 h. The solvent was evaporated in vacuo to obtain the crude product. The crude reaction mixture was purified by column chromatography on silica gel (5% to 10% MeOH:DCM) to get the desired product prop-2-yn-1-yl (4-((((4-nitrophenoxy)carbonyl)oxy)methyl)phenyl)carbamate (453 mg, 1.22 mmol, 63%).

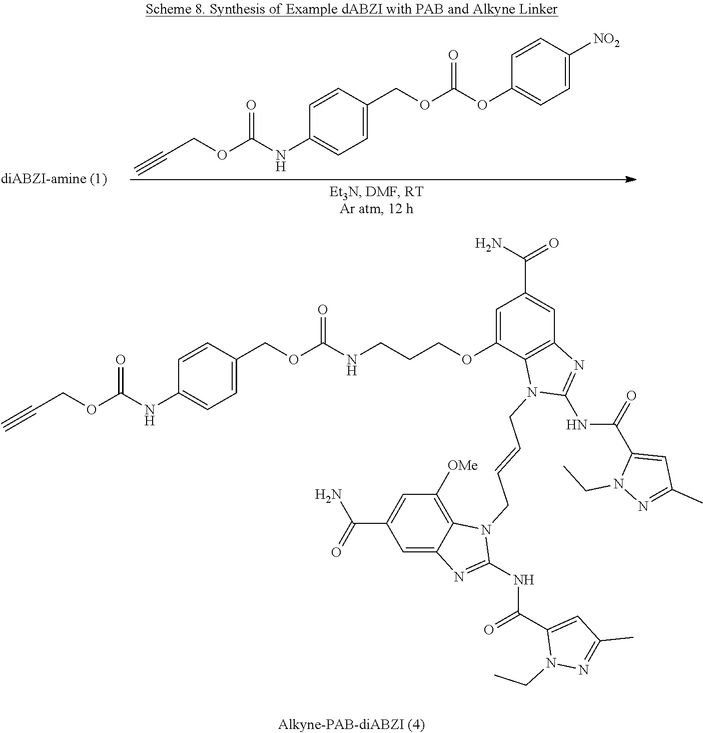 [SMILES: C#CCOC(=O)Nc1ccc(COC(=O)Oc2ccc([N+](=O)[O-])cc2)cc1 ; C#CCOC(=O)Nc1ccc(COC(=O)NCCCOc2cc(C(N)=O)cc3nc(NC(=O)c4cc(C)nn4CC)n(C/C=C/Cn4c(NC(=O)c5cc(C)nn5CC)nc5cc(C(N)=O)cc(OC)c54)c23)cc1]

A solution of diABZI-amine (1) (126 mg, 0.11 mmol, 1 eq) and Hunig's base (98 μL, 0.56 mmol, 5 eq) in DMF (5 mL) stirred under argon atmosphere at room temperature. A solution of crude p-nitrophenol ester (50 mg, 0.13 mmol, 1.2 eq) in DMF (2 mL) was added dropwise to the reaction mixture. The reaction mixture was stirred for 6 h. The diethyl ether added to precipitate out the solid product to obtain the desired product (4) as an light yellow solid (87 mg, 0.09 mmol, 76%).

Alkyne-PAB-diABZI (4) HRMS (ESMS) Calculated for C₅₀H₅₄N₁₄O₁₀ [M+H]⁺: 1011.4226, found 1011.4193.

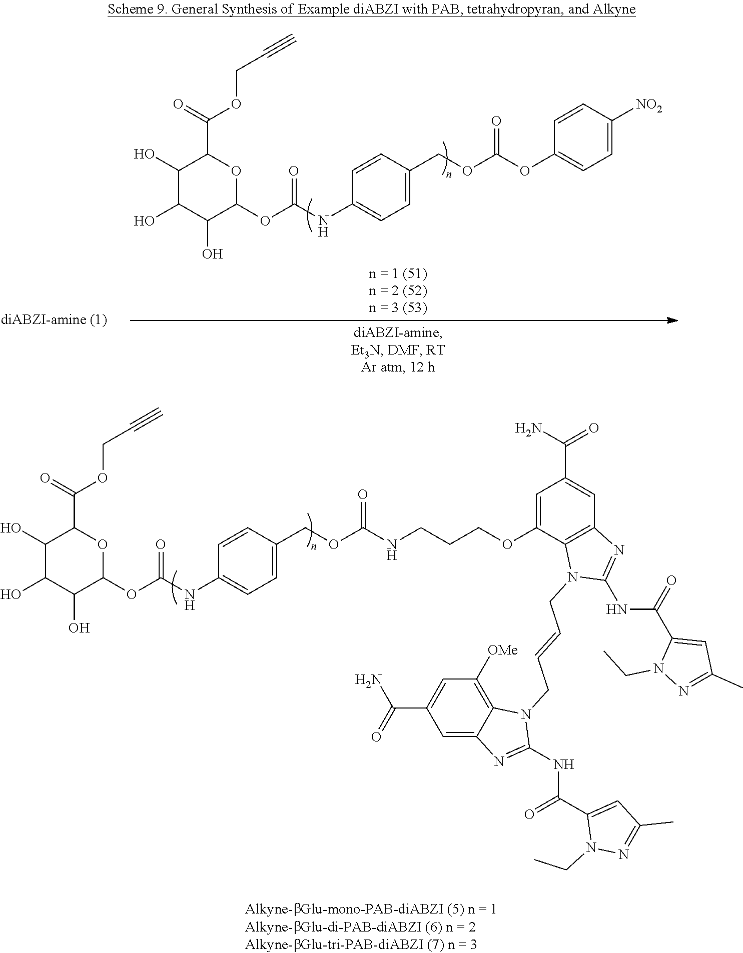

A solution of diABZI-amine (1) (1 eq) and Hunig's base (5 eq) in DMF stirred under argon atmosphere at room temperature. A solution of crude p-nitrophenol ester (1.2 eq) in DMF was added dropwise to the reaction mixture. The reaction mixture was stirred for 6 h. The diethyl ether added to precipitate out the solid product to obtain the desired product.

Alkyne-βGlu-mono-PAB-diABZI (5) (yield 41%) HRMS (ESMS) Calculated for C₅₆H₆₂N₁₄O₁₆ [M+H]₊: 1187.4546, found 1187.4511.

Alkyne-βGlu-di-PAB-diABZI (6) (yield 94%) HRMS (ESMS) Calculated for C₆₄H₆₉N₁₅O₁₈ [M+H]⁺: 1336.5023, found 1336.4984.

Alkyne-βGlu-tri-PAB-diABZI (7) (yield 83%) HRMS (ESMS) Calculated for C₇₂H₇₆N₁₆O₂₀ [M+H]⁺: 1485.5500, found 1485.5446.

### 2. Biological Evaluation of the Compounds

THP-1 Dual reporter cells were utilized to evaluate interferon production by compounds 4-7. Functionalization of compound 4 resulted in a decrease in STING activation; however, an increase in spacer length appeared to correlate with improved STING activation when comparing compounds 5-7.

### Example 4

Synthesis and Functional Activity of diABZI with Cyclooctyne Functionalized Linkers

### 1. Synthesis of Compounds

### Abbreviations

Et₃N is triethylamine;
DMF is dimethyl formamide;
MeOH is methanol;
DIPEA is N,N diisopropylethylamine;
HRMS is high-resolution mas spectroscopy;
NMR is nuclear magnetic resonance;
atm is atmospheric pressure;
eq. or equiv. is equivalents;
rt or RT is room temperature;
hr or h is hour; and
min is minutes.

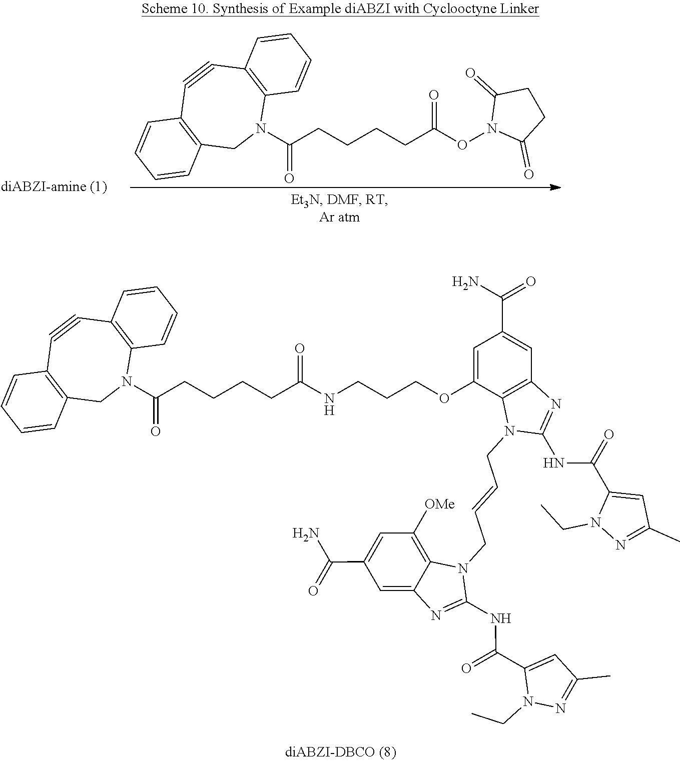 [SMILES: O=C(CCCCC(=O)N1Cc2ccccc2C#Cc2ccccc21)ON1C(=O)CCC1=O ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCNC(=O)CCCCC(=O)N3Cc4ccccc4C#Cc4ccccc43)c21]

A solution of diABZI-amine (1) (414 mg, 0.37 mmol, 1 eq) and triethyl amine (0.25 mL, 1.85 mmol, 5 eq) in DMF (5 mL) stirred under argon atmosphere at room temperature. A solution of activated NHS-ester (175 mg, 0.40 mmol, 1.1 eq) in DMF (4 mL) was added dropwise to the reaction mixture. The reaction mixture was stirred for overnight. The solvent DMF was evaporated in vacuo to obtain the crude desired product. The crude solid was purified over silica gel chromatography (DCM:MeOH 0-25%) to obtain the desired product diABZI-DBCO (8) as an light pink solid (153 mg, 0.14 mmol, 38%).

diABZI-DBCO (8). HRMS (ESMS) Calculated for C₅₉H₆₂N₁₄O₈[M+Na]⁺: 1117.4773, found 1117.4772.

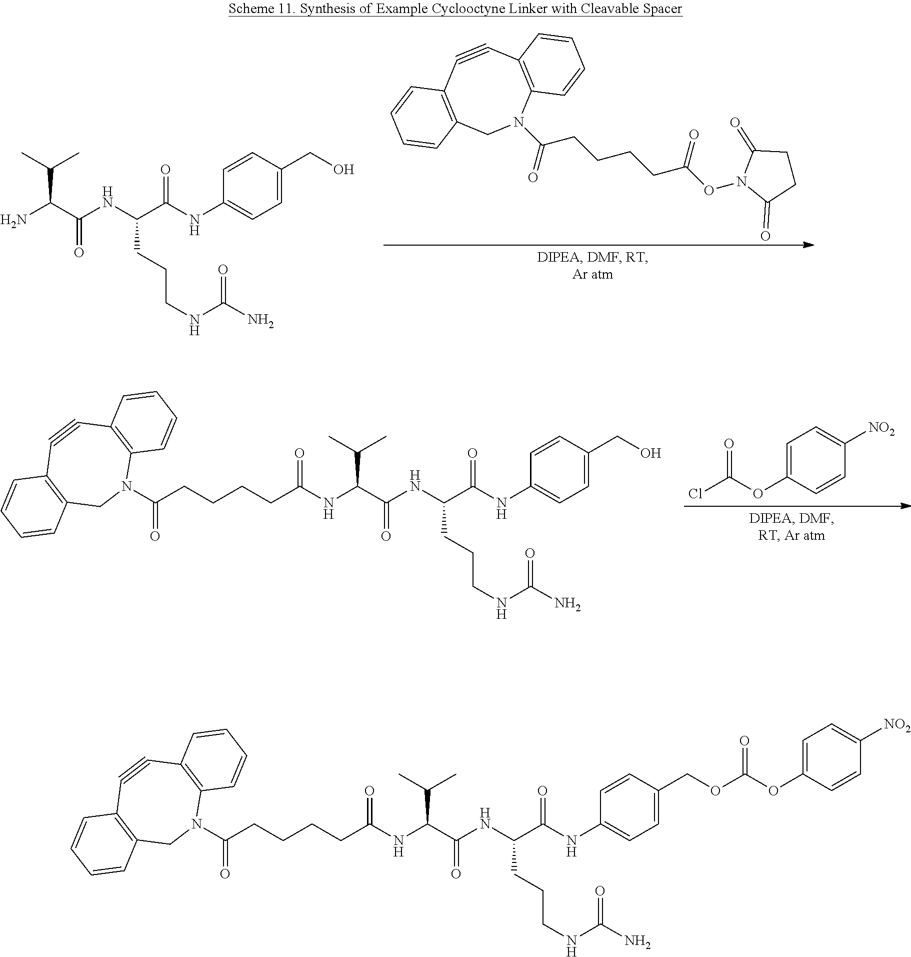 [SMILES: CC(C)[C@H](N)C(=O)N[C@@H](CCCNC(N)=O)C(=O)Nc1ccc(CO)cc1 ; O=C(CCCCC(=O)N1Cc2ccccc2C#Cc2ccccc21)ON1C(=O)CCC1=O ; CC(C)[C@H](NC(=O)CCCCC(=O)N1Cc2ccccc2C#Cc2ccccc21)C(=O)N[C@@H](CCCNC(N)=O)C(=O)Nc1ccc(CO)cc1 ; O=C(Cl)Oc1ccc([N+](=O)[O-])cc1 ; CC(C)[C@H](NC(=O)CCCCC(=O)N1Cc2ccccc2C#Cc2ccccc21)C(=O)N[C@@H](CCCNC(N)=O)C(=O)Nc1ccc(COC(=O)Oc2ccc([N+](=O)[O-])cc2)cc1]

To a stirred solution of (S)-2-((S)-2-amino-3-methylbutanamido)-N-(4-(hydroxymethyl)phenyl)-5-ureidopentanamide (400 mg, 1.05 mmol, 1 eq) and Hunig's base (550 μL, 3.16 mmol, 3 eq) in DMF 5 mL dropwise add solution of activated NHS ester (500 mg, 1.16 mmol, 1.1 eq) in DMF (2 mL) stir overnight under argon atmosphere. The diethyl ether added to precipitate out the solid product to obtain the desired product with terminal benzyl alcohol as an intermediate (702 mg, 1.10 mmol, 96%). To the stirred solution of intermediate alcohol (550 mg, 0.79 mmol, 1 eq) and Hunig's base (689 μL, 3.95 mmol, 5 eq) in DMF (5 mL) add 4-nitrophenyl carbonochloridate (319 mg, 1.58 mmol, 2.0 eq) and stir overnight. The diethyl ether added to precipitate out the solid product to obtain as crude product. The crude solid was purified over silica gel chromatography (DCM:MeOH 0-25%) to obtain the desired product (113 mg, 0.13 mmol, 17%).

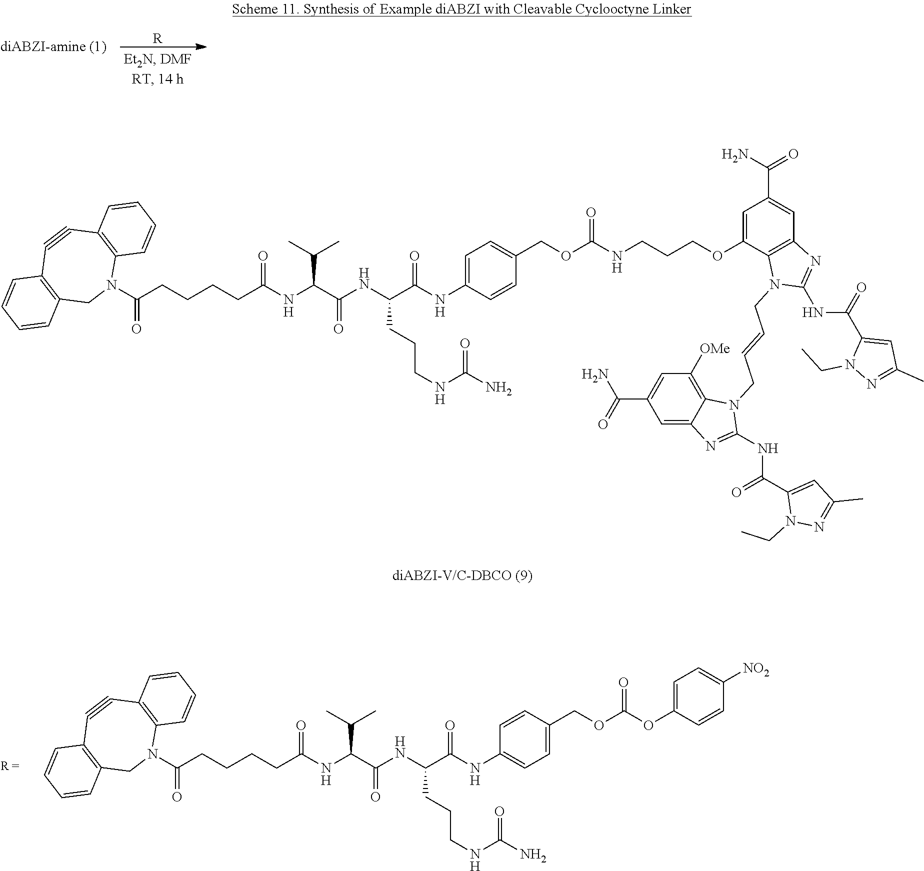

A solution of diABZI-amine (1) (140 mg, 0.12 mmol, 1 eq) and Hunig's base (0.11 mL, 0.62 mmol, 5 eq) in DMF (3 mL) stirred under argon atmosphere at room temperature. A solution of activated ester (113 mg, 0.13 mmol, 1.05 eq) in DMF (2 mL) was added dropwise to the reaction mixture. The reaction mixture was stirred for overnight. The solvent DMF was evaporated in vacuo to obtain the crude desired product. The crude solid was purified over silica gel chromatography (DCM:MeOH 0-25%) to obtain the desired product diABZI-V/C-DBCO (9) as an light pink solid (170 mg, 0.11 mmol, 86%).

diABZI-V/C-DBCO (9). HRMS (ESMS) Calculated for C₇₈H₈₉N₁₉O₁₃ [M+Na]⁺: 1522.6785, found 1522.6821.

### 2. Biological Evaluation of Compounds

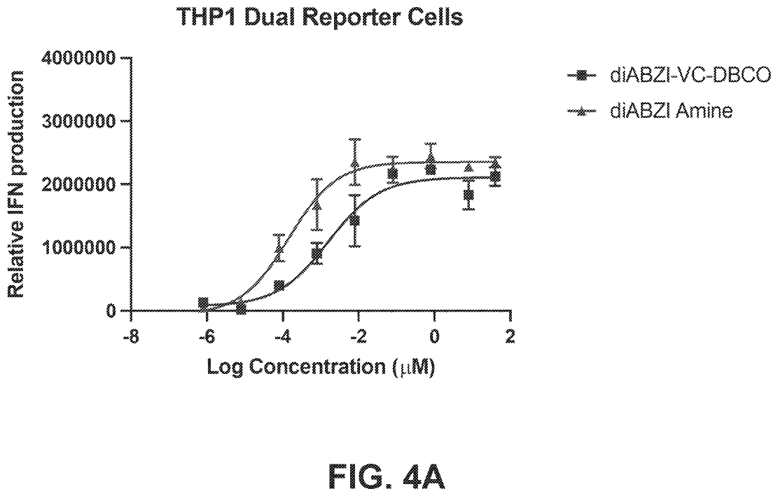

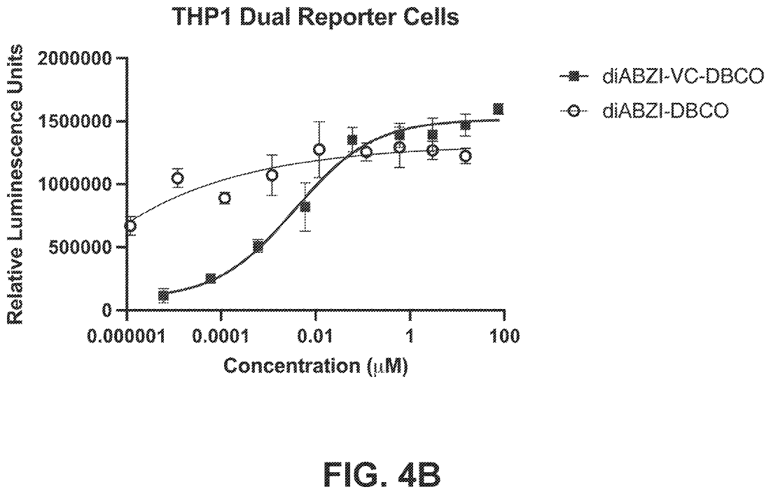

Although maleimide-functionalized STING agonists hold value for efficient conjugation to thiolated drug carriers, the use of thiols in polymer backbones can lead to crosslinking and undesired morphological effects. Dibenzocyclooctyne (DBCO) functionalized diABZI variants that would allow for conjugation to azide groups on polymers via copper-free click chemistry. diABZI-DBCO compounds can be synthesized with or without cleavable linkers. Compound (9) is an example of a variant with an enzyme cleavable spacer. Both stable (8) and enzyme cleavable (9) variants elicit STING activation as demonstrated in THP-1 Dual™ reporter cell lines, using with the stable variant serving as a more potent agonist (FIG. 4A, 4B).

### Example 5

Synthesis and Functional Activity of diABZI with CTA Functionalized Linkers

### 1. Synthesis of Compounds

### Abbreviations

Et₃N is triethylamine;
DMF is dimethyl formamide;
DIPEA is N,N diisopropylethylamine;
Hunig's base is DIPEA;
PNP is p-nitrophenol;
DCC is N,N′-dicyclohexylcarbodiimide;
DMAP is 4-dimethylaminopyridine;
EDCl is 1-ethyl-3-(3-dimethylaminopropyl)carbodiimide;
EtOAc is ethyl acetate;
PFP is pentafluorophenyl esters;
ECT is 4-cyano-4-(ethylsulfanylthiocarbonyl) sulfanylvpentanoic acid;
CTA is chain transfer agent;
DCM is dichloromethane;
MeOH is methanol;
Na₂S₂O₄ is sodium dithionate;
TLC is thin-layer chromatography;
HRMS is high-resolution mas spectroscopy;
NMR is nuclear magnetic resonance;
atm is atmospheric pressure;
eq. or equiv. is equivalents;
rt or RT is room temperature;
hr or h is hour; and
min is minutes.

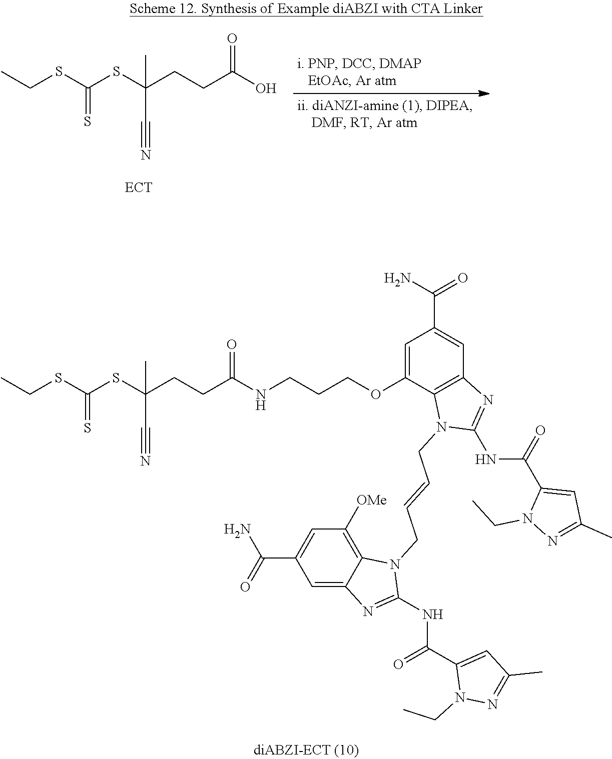 [SMILES: CCSC(=S)SC(C)(C#N)CCC(=O)O ; CCSC(=S)SC(C)(C#N)CCC(=O)NCCCOc1cc(C(N)=O)cc2nc(NC(=O)c3cc(C)nn3CC)n(C/C=C/Cn3c(NC(=O)c4cc(C)nn4CC)nc4cc(C(N)=O)cc(OC)c43)c12]

A solution of 4-cyano-4-(((ethylthio)carbonothioyl)thio)pentanoic acid (100 mg, 0.38 mmol, 1 eq), p-nitrophenol (53 mg, 0.38 mmol, 1 eq) and DMAP (5 mg, 0.038 mmol, 0.1 eq) in EtOAc (4 mL) was maintained at 0° C. under argon atmosphere. A solution of DCC (82 mg, 0.40 mmol, 1.05 eq) in EtOAc (4 mL) was added dropwise. The reaction mixture was allowed to warm to rt and stirred overnight for 18 h. After consumption of the starting material, as judged by the TLC analyses, the reaction mixture was filtered through celite. The solvent was evaporated in vacuo to obtain the activated ester as a crude product. The crude product was analyzed by 1H NMR spectroscopy and was used without further purification. ¹H NMR (400 MHz, Chloroform-d) δ 8.28 (d, J=9.2 Hz, 2H), 7.31 (d, J=9.2 Hz, 2H), 3.36 (q, J=7.4 Hz, 2H), 2.95 (t, J=7.9 Hz, 2H), 2.70-2.46 (m, 2H), 1.95 (s, 3H), 1.37 (t, J=7.4 Hz, 3H).

A solution of diABZI-amine (1) (300 mg, 0.27 mmol, 1 eq) and Hunig's base (0.23 mL, 1.35 mmol, 5 eq) in DMF (5 mL) stirred under argon atmosphere at room temperature. A solution of crude p-nitrophenol ester (144 mg, 0.37 mmol, 1.4 eq) in DMF (4 mL) was added dropwise to the reaction mixture. The reaction mixture was stirred for 6 h. The solvent DMF was evaporated in vacuo to obtain the crude desired product. The crude solid was purified over silica gel chromatography (DCM:MeOH 0-25%) to obtain the desired product diABZI-ECT (10) as an light yellow solid (200 mg, 0.19 mmol, 73%).

diABZI-ECT (10) ¹H NMR (400 MHz, DMSO) δ 8.04 (t, J=5.2 Hz, 1H), 7.98 (broad s, 2H), 7.56 (broad s, 2H), 7.35 (broad s, 2H), 7.31 (d, J=5.9 Hz, 2H), 6.56 (d, J=8.5 Hz, 2H), 4.58-4.52 (m, 4H), 4.38-4.34 (m, 4H), 4.10 (t, J=5.90 Hz, 2H), 3.85 (s, 3H), 3.19-3.14 (m, 2H), 2.35-2.20 (m, 4H), 2.09 (s, 6H), 1.86 (broad s, 6H), 1.80 (s, 3H), 1.29 (t, J=7.0 Hz, 6H), 1.25 (t, J=7.3 Hz, 3H). HRMS (ESMS) Calculated for C₄₇H₅₆N₁₄O₇S₃ [M+H]⁺: 1025.3697, found 1025.3685.

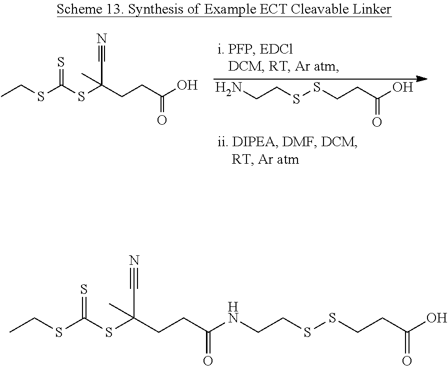 [SMILES: CCSC(=S)SC(C)(C#N)CCC(=O)O ; NCCSSCCC(=O)O ; CCSC(=S)SC(C)(C#N)CCC(=O)NCCSSCCC(=O)O]

A solution of 4-cyano-4-(((ethylthio)carbonothioyl)thio)pentanoic acid (2.4 g, 9.11 mmol, 1 eq), and EDCl (2.09 g, 10.9 mmol, 1.2 eq) in DCM (30 mL) was maintained at rt under argon atmosphere. A solution of pentafluorophenol (1.67 g, 9.11 mmol, 1 eq) in DCM (10 mL) was added dropwise. The reaction mixture was stirred for 2 h at rt. The solution of 3-((2-aminoethyl)disulfaneyl)propanoic acid hydrochloride (2.18 g, 10.02 mmol, 1.1 eq) in DCM 10 mL added dropwise followed by addition of Hunig's base (8 mL, 45.5 mmol, 5 eq) and stirred for 8 h. After consumption of the starting material, as judged by the TLC analyses, the organic layer was washed with 0.1 M HCl (2×60 mL). The organic layer was dried over Na₂S₂O₄ and evaporated in vacuo to obtain the crude product. The crude reaction mixture was purified by column chromatography on silica gel (0% to 5% MeOH:DCM) to get the desired product 6-cyano-6-methyl-9-oxo-4-thioxo-3,5,13,14-tetrathia-10-azaheptadecan-17-oic acid as a thick yellow oil (2.2 g, 5.15 mmol, 57%).

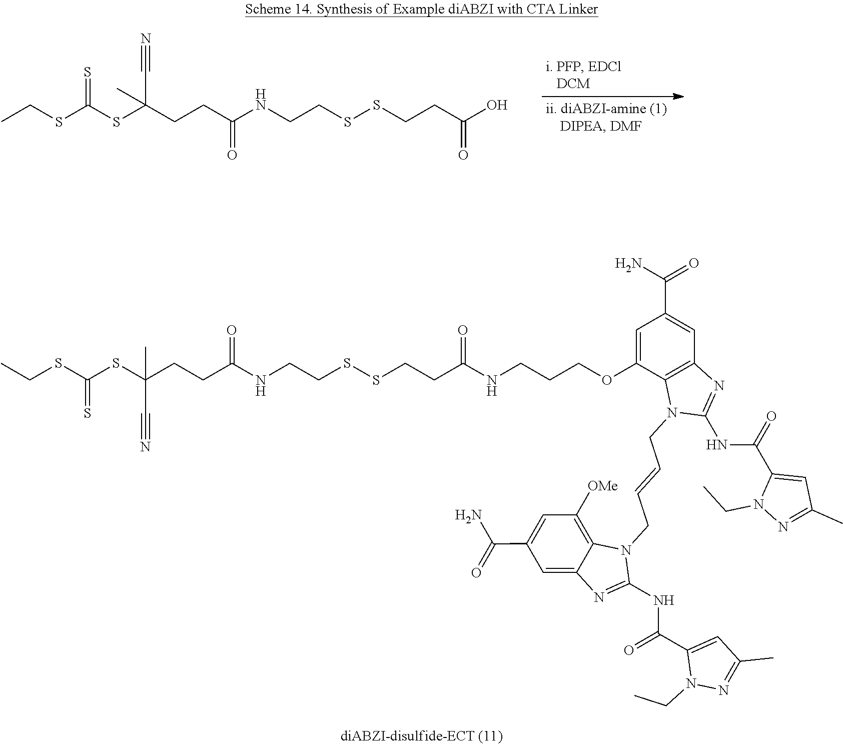 [SMILES: CCSC(=S)SC(C)(C#N)CCC(=O)NCCSSCCC(=O)O ; CCSC(=S)SC(C)(C#N)CCC(=O)NCCSSCCC(=O)NCCCOc1cc(C(N)=O)cc2nc(NC(=O)c3cc(C)nn3CC)n(C/C=C/Cn3c(NC(=O)c4cc(C)nn4CC)nc4cc(C(N)=O)cc(OC)c43)c12]

A solution of 6-cyano-6-methyl-9-oxo-4-thioxo-3,5,13,14-tetrathia-10-azaheptadecan-17-oic acid (80 mg, 0.18 mmol, 1 eq) and EDCl (43 mg, 0.22 mmol, 1.2 eq) in DCM (3 mL) was maintained at rt under argon atmosphere. A solution of pentafluorophenol (34 mg, 0.18 mmol, 1 eq) in DCM (1 mL) was added dropwise. The reaction mixture was stirred for 2 h at rt. The solution of diABZI amine (1) (189 mg, 0.16 mmol, 0.9 eq) and Hunig's base (163 μL, 0.93 mmol, 5 eq) in DMF 3 mL added dropwise to the reaction mixture and stirred for overnight. The diethyl ether added to precipitate out the solid product to obtain as crude product. The crude solid was purified over silica gel chromatography (DCM:MeOH 0-25%) to obtain the desired product diABZI-SS-ECT (11) as yellow solid (122 mg, 0.10 mmol, 55%).

diABZI-SS-ECT (11). ¹H NMR (400 MHz, MeOD) δ 7.57 (d, J=13.0 Hz, 2H), 7.25 (d, J=20.0 Hz, 2H), 6.57 (d, J=17.2 Hz, 2H), 5.91-5.79 (m, 2H), 5.06-4.99 (m, 4H), 4.63-4.54 (m, 4H), 3.94 (t, J=6.0 Hz, 2H), 3.75 (s, 3H), 3.45 (d, J=6.5 Hz, 2H), 3.22 (d, J=6.9 Hz, 2H), 2.90 (d, J=6.9 Hz, 2H), 2.79 (d, J=6.6 Hz, 2H), 2.54 (t, J=7.0 Hz, 2H), 2.49-2.41 (m, 3H), 2.35-2.29 (m, 1H), 2.20 (s, 3H), 2.18 (s, 3H), 1.82 (s, 3H), 1.76 (p, J=6.2 Hz, 2H), 1.37-1.27 (m, 9H).

### 2. Biological Evaluation of Compounds

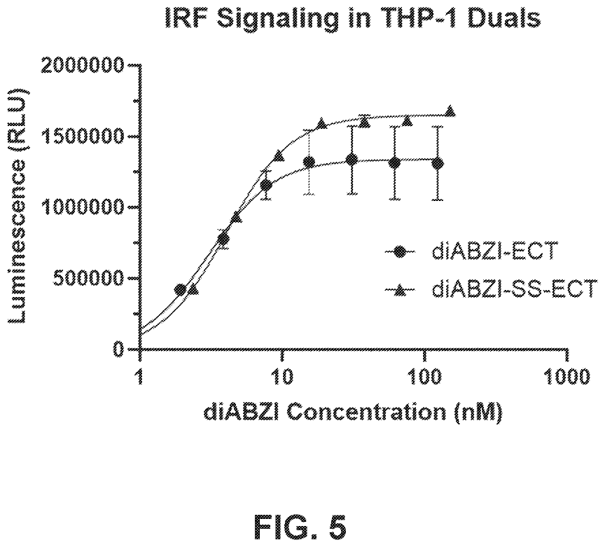

Another method of covalently ligating a diABZI variant to a polymer backbone is through a functionalized chain transfer agent (CTA) that allows for direct chain extension from the STING agonist using RAFT polymerization. diABZI-ECT can then be used as a chain transfer agent for RAFT polymerization of poly(acrylamides), poly(methacrylamides), poly(acrylates), and poly(methacrylates) and combinations thereof. Therefore, we synthesized diABZI-ECT from the chain transfer agent, (ethylsulfanylthiocarbonyl)-sulfanylvpentanoic acid (ECT). The carboxylic acid was synthesized using a literature protocol and confirmed by using 1H NMR. The carboxylic acid was then converted into activated PNP-ester using DCC-DMAP coupling and treated with diABZI-amine (1) to obtain the desired product as diABZI-ECT (10). diABZI-ECT can also be synthesized with or without a cleavable linker; diABZI-SS-ECT (11) is an example of a variant with a disulfide linker that can be cleaved under reducing conditions in cells or tissues. Using THP-1 Dual™ reporter cell lines, diABZI-ECT (10) and diABZI-SS-ECT (11) elicit similar in vitro STING activation and exhibit EC₅₀ values of 3.06 nM and 4.09 nM, respectively (FIG. 5).

### Example 6

Synthesis and Functional Activity of diABZI with Methylacrylate Functionalized Linkers

### 1. Synthesis of Compounds

### Abbreviations

Et₃N is triethylamine;
DMF is dimethyl formamide;
DMSO is dimethyl sulfoxide;
DCM is dichloromethane;
MeOH is methanol;
HRMS is high-resolution mas spectroscopy;
NMR is nuclear magnetic resonance;
atm is atmospheric pressure;
eq. or equiv. is equivalents;
rt or RT is room temperature;
hr or h is hour; and
min is minutes.

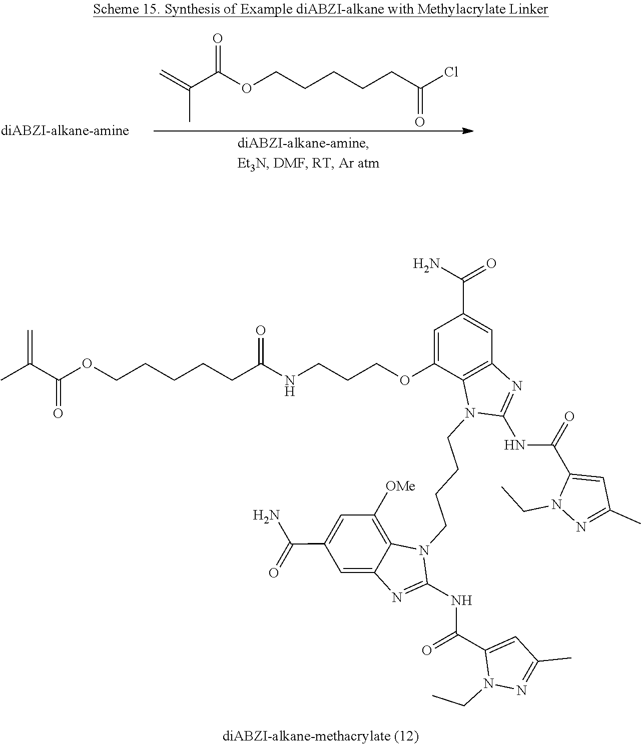 [SMILES: C=C(C)C(=O)OCCCCCC(=O)Cl ; C=C(C)C(=O)OCCCCCC(=O)NCCCOc1cc(C(N)=O)cc2nc(NC(=O)c3cc(C)nn3CC)n(CCCCn3c(NC(=O)c4cc(C)nn4CC)nc4cc(C(N)=O)cc(OC)c43)c12]

A solution of diABZI-alkane-amine (300 mg, 0.27 mmol, 1 eq) and triethyl amine (0.15 mL, 1.06 mmol, 4 eq) in DMF (6 mL) were maintained under argon atmosphere at room temperature. A solution of 6-chloro-6-oxohexyl methacrylate (70 mg, 0.35 mmol, 1.2 eq) in DMF (2 mL) was added dropwise to the reaction mixture. The reaction mixture was stirred for overnight. The diethyl ether added to precipitate out the solid product to obtain as crude product. The crude solid was purified over silica gel chromatography (DCM:MeOH 0-25%) to obtain the desired product as off-white solid (211 mg, 0.22 mmol, 82%).

diABZI-alkane-methacrylate (12). ¹H NMR (400 MHz, DMSO) δ 7.97 (broad s, 2H), 7.78 (t, J=5.4 Hz, 1H), 7.55 (d, J=3.8 Hz, 2H), 7.34-7.29 (m, 4H), 6.55 (d, J=8.2 Hz, 2H), 5.96 (s, 1H), 5.61-5.59 (m, 1H), 4.58-4.52 (m, 4H), 4.39-4.32 (m, 4H), 4.08 (t, J=6.0 Hz, 1H), 3.84 (s, 3H), 3.17-3.12 (m, 2H), 3.09-3.04 (m, 6H), 2.08 (s, 3H), 2.07 (s, 3H), 1.98 (t, J=7.2 Hz, 6H), 1.87-1.75 (m, 9H), 1.57-1.40 (m, 4H), 1.28 (t, J=6.8 Hz, 6H). HRMS (ESMS) Calculated for C₄₈H₆₁N₁₃O₉[M+H]⁺: 964.4793, found 964.4793.

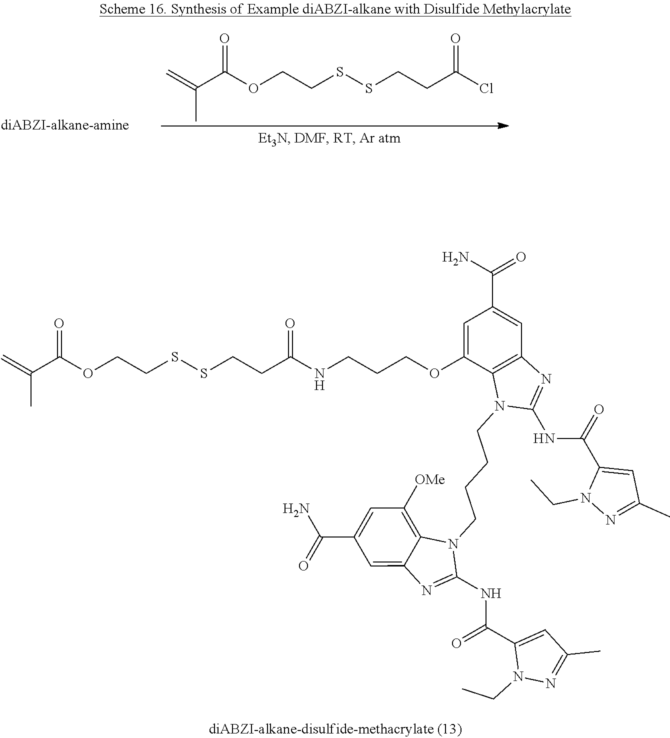 [SMILES: C=C(C)C(=O)OCCSSCCC(=O)Cl ; C=C(C)C(=O)OCCSSCCC(=O)NCCCOc1cc(C(N)=O)cc2nc(NC(=O)c3cc(C)nn3CC)n(CCCCn3c(NC(=O)c4cc(C)nn4CC)nc4cc(C(N)=O)cc(OC)c43)c12]

A solution of diABZI-alkane-amine (410 mg, 0.36 mmol, 1 eq) and triethyl amine (0.20 mL, 1.45 mmol, 4 eq) in DMF (6 mL) were maintained under argon atmosphere at room temperature. A solution of 2-((3-chloro-3-oxopropyl)disulfaneyl)ethyl methacrylate (117 mg, 0.43 mmol, 1.2 eq) in DMF (2 mL) was added dropwise to the reaction mixture. The reaction mixture was stirred for overnight. The diethyl ether added to precipitate out the solid product to obtain as crude product. The crude solid was purified over silica gel chromatography (DCM:MeOH 0-25%) to obtain the desired product as off-white solid (300 mg, 0.31 mmol, 85%).

diABZI-alkane-disulfide-methacrylate (13). HRMS (ESMS) Calculated for C₄₇H₅₉N₁₃O₉S₂ [M+H]⁺: 1014.4078, found 1014.4077.

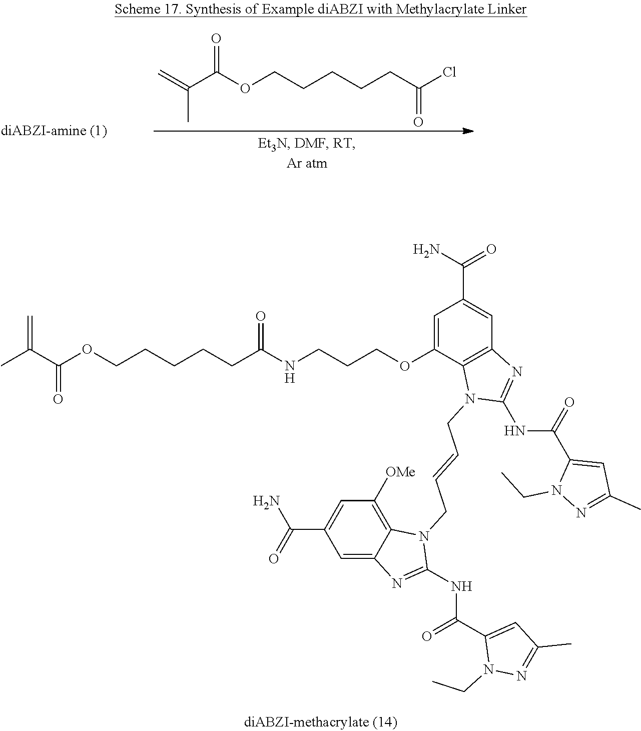 [SMILES: C=C(C)C(=O)OCCCCCC(=O)Cl ; C=C(C)C(=O)OCCCCCC(=O)NCCCOc1cc(C(N)=O)cc2nc(NC(=O)c3cc(C)nn3CC)n(C/C=C/Cn3c(NC(=O)c4cc(C)nn4CC)nc4cc(C(N)=O)cc(OC)c43)c12]

A solution of diABZI-amine (1) (300 mg, 0.27 mmol, 1 eq) and triethyl amine (0.15 mL, 1.06 mmol, 4 eq) in DMF (6 mL) were maintained under argon atmosphere at room temperature. A solution of 6-chloro-6-oxohexyl methacrylate (76 mg, 0.35 mmol, 1.3 eq) in DMF (2 mL) was added dropwise to the reaction mixture. The reaction mixture was stirred for overnight. The diethyl ether added to precipitate out the solid product to obtain as crude product. The crude solid was purified over silica gel chromatography (DCM:MeOH 0-25%) to obtain the desired product as off-white solid (212 mg, 0.22 mmol, 82%).

diABZI-methacrylate (14). HRMS (ESMS) Calculated for C₄₈H₅₉N₁₃O₉[M+H]⁺: 962.4637, found 962.4646.

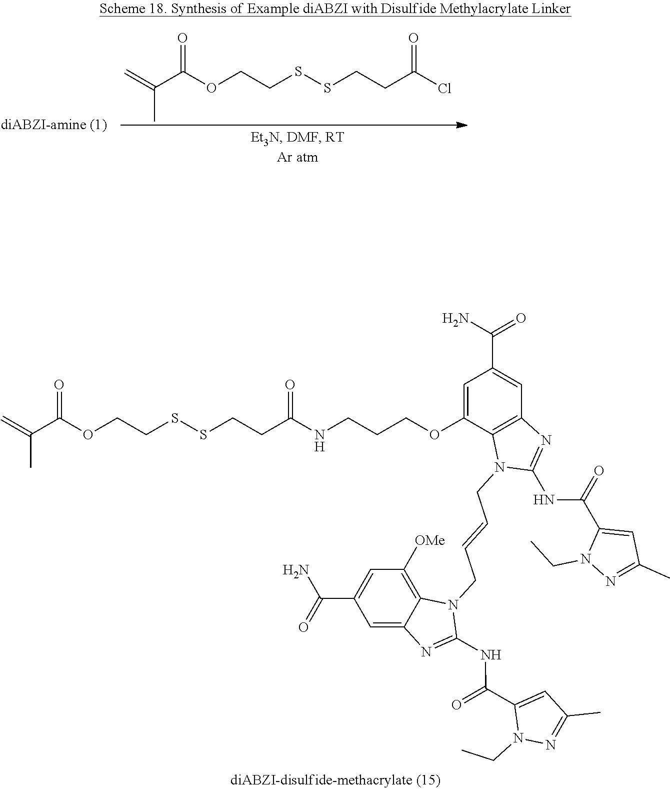 [SMILES: C=C(C)C(=O)OCCSSCCC(=O)Cl ; C=C(C)C(=O)OCCSSCCC(=O)NCCCOc1cc(C(N)=O)cc2nc(NC(=O)c3cc(C)nn3CC)n(C/C=C/Cn3c(NC(=O)c4cc(C)nn4CC)nc4cc(C(N)=O)cc(OC)c43)c12]

A solution of diABZI-amine (1) (300 mg, 0.27 mmol, 1 eq) and triethyl amine (0.15 mL, 1.06 mmol, 4 eq) in DMF (6 mL) were maintained under argon atmosphere at room temperature. A solution of 2-((3-chloro-3-oxopropyl)disulfaneyl)ethyl methacrylate (86 mg, 0.32 mmol, 1.2 eq) in DMF (2 mL) was added dropwise to the reaction mixture. The reaction mixture was stirred for overnight. The diethyl ether added to precipitate out the solid product to obtain as crude product.

The crude solid was purified over silica gel chromatography (DCM:MeOH 0-25%) to obtain the desired product as off-white solid (260 mg, 0.25 mmol, 96%).

diABZI-disulfide-methacrylate (15). HRMS (ESMS) Calculated for C₄₇H₅₇N₁₃O₉S₂ [M+H]⁺: 1012.3922, found 1012.3931.

### 2. Biological Evaluation of Compounds

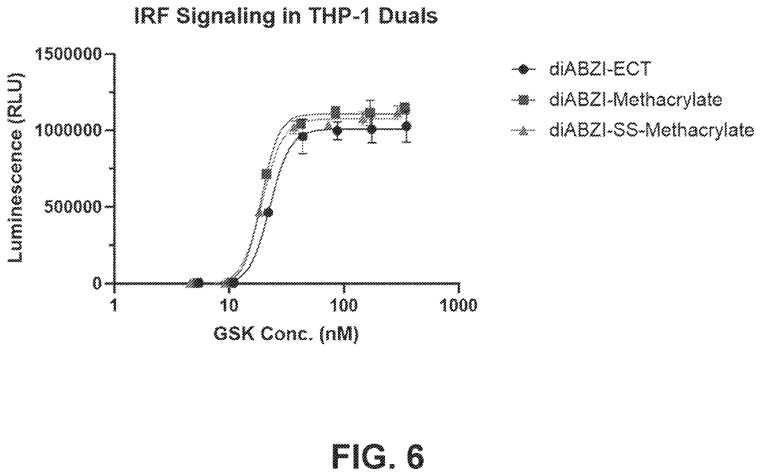

While the previous methods focused on developing compounds that could be directly incorporated into a polymer backbone through modifying a chain transfer agent, we next sought to develop diABZI-functionalized monomers that could be directly rafted into a polymer backbone, allowing for more than one drug per polymer chain. We first sought to develop less complex diABZI-like monomers (12 and 13), which have an alkane connection within the drug rather than the typical alkene. However, the addition of the alkene is known to result in a more potent drug. Therefore, once we confirmed that synthesis for (12) and (13) was successful, we synthesized compounds (14) and (15). In vitro activity of these variants was tested in THP-1 Dual™ reporter cell lines. Compounds (10), (14), and (15) had nearly identical STING activation, demonstrating that the monomers are as active as diABZI-ECT and that the addition of the cleavable disulfide linker is not necessary for STING activation compared to the non-cleavable version (FIG. 6).

### Example 7

Synthesis and Functional Activity of diABZI Conjugated to a Fluorescent Molecule

### 1. Synthesis of Compounds

### Abbreviations

Et₃N is triethylamine;
DMF is dimethyl formamide;
DIPEA is N,N diisopropylethylamine;
Hunig's base is DIPEA;
DCM is dichloromethane;
DMSO is dimethyl sulfoxide;
HRMS is high-resolution mas spectroscopy;
NMR is nuclear magnetic resonance;
atm is atmospheric pressure;
eq. or equiv. is equivalents;
rt or RT is room temperature;
hr or h is hour; and
min is minutes.

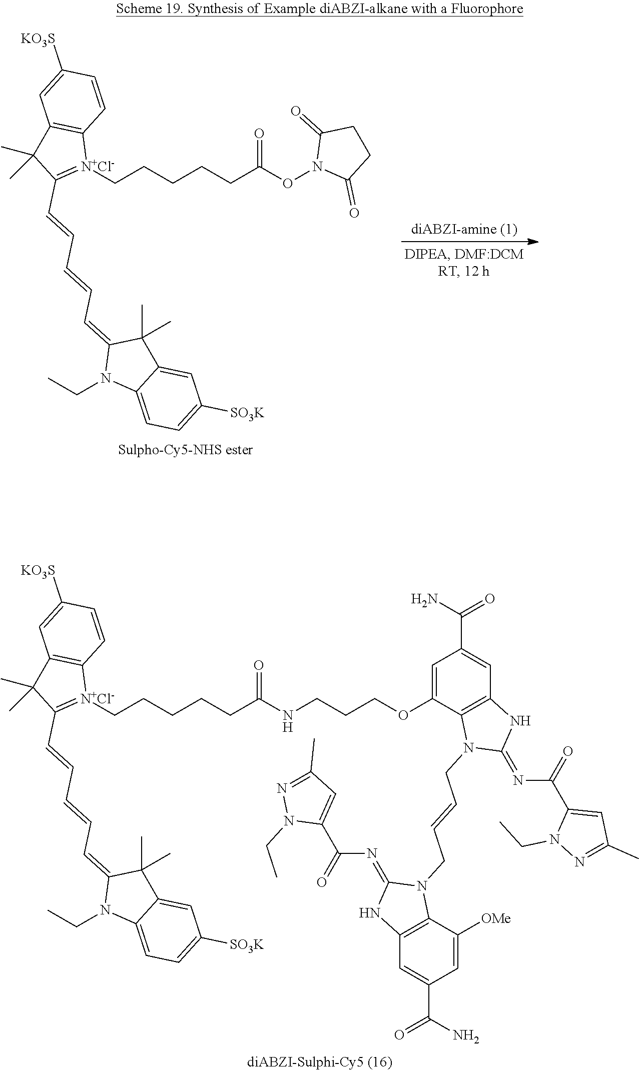

To a stirred solution of compound (1) (18 mg, 16 μmol, 1.0 eq) and Hunig's base (8.4 μL, 48 μmol, 3 eq) in DMF (1.5 mL) dropwise add solution of activated NHS ester compound (23) in DMF (1.0 mL). The reaction mixture stirred overnight and precipitated by adding diethyl to obtain the desired product (16) as a blue solid (17.2 mg, 11.2 μmol, 70%).

diABZI-sulphi-Cy5 (16). ¹H NMR (600 MHz, DMSO) δ 8.39-8.34 (m, 3H), 7.82-7.81 (m, 3H), 7.65-7.62 (m, 2H, 6.78-6.73 (m, 3H), 6.33-6.28 (m, 2H), 4.94-4.87 (m, 4H), 4.55-4.53 (m, 4H), 3.85 (s, 3H), 3.19-3.14 (m, 2H), 2.35-2.20 (m, 4H), 2.09 (s, 6H), 1.69 (broad s, 6H), 1.59-1.54 (m, 2H), 1.27 (t, J=7.0 Hz, 6H).

### Example 8

Synthesis and Functional Activity PEG-diABZI Conjugates with Stable and V/C Linkers

### 1. Synthesis of the Conjugates

### Abbreviations

Et₃N is triethylamine;
DMF is dimethyl formamide;
DCM is dichloromethane;
MeOH is methanol;
DMSO is dimethyl sulfoxide;
NMM is N-methyl morpholine;
HRMS is high-resolution mas spectroscopy;
NMR is nuclear magnetic resonance;
atm is atmospheric pressure;
eq. or equiv. is equivalents;
rt or RT is room temperature;
hr or h is hour; and
min is minutes.

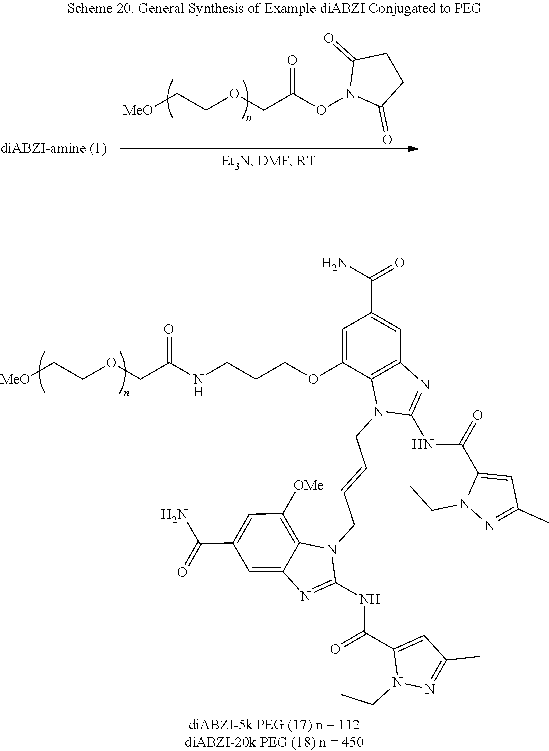

diABZI-5k PEG (17) To a stirred solution of diABZI-amine (1) (81 mg, 72 μmol, 1.2 eq) and Et₃N (42 μL, 300 μmol, 5 eq) in DMF (2 mL) add solution of Methoxy-PEG(5k)-NHS ester (300 mg, 60 μmol, 1 eq) in 1:1 DCM:DMF 4 mL and stir overnight. The desired compound (17) was purified by dialysis (3 kDa MWCO) against (1:1) DCM:MeOH (2×), acetone (2×) followed by distilled water (2×). The desired purified product (180 mg, 31.2 μmol, 52%) was lyophilized and characterized by ¹H NMR. ¹H NMR (400 MHz, DMSO) δ 7.95 (broad s, 2H), 7.94 (broad s, 3H), 7.73 (t, 1H), 7.64 (s, 2H), 7.31-7.28 (m, 5H), 6.5 (d, 2H), 5.87-5.82 (m, 2H), 4.96-4.89 (m, 4H), 4.53-4.50 (m, 4H), 3.97 (t, J=6.1 Hz, 2H), 3.81 (s, 3H, terminal methoxy of PEG), 3.72 (s, 3H), 3.51 (broad s, 462H, —CH₂—CH₂— of PEGMA), 2.10 (s, 3H), 2.09 (s, 3H), 1.70 (p, J=6.1 Hz, 2H), 1.28-1.24 (m, 6H).

diABZI-20k PEG (18) To a stirred solution of compound 1 (34 mg, 30 μmol, 1.2 eq) and Et₃N (17.5 μL, 125 μmol, 5 eq) in DMF (1.5 mL) add solution of Methoxy-PEG(20k)-NHS ester (500 mg, 25 μmol, 1 eq) in 1:1 DCM:DMF 5 mL and stir overnight. The desired compound (18) was purified by dialysis (3 kDa MWCO) against (1:1) DCM:MeOH (2×), acetone (2×) followed by distilled water (2×). The desired purified product was lyophilized to obtain white fluffy powder (393 mg, 19 μmol, 76%).

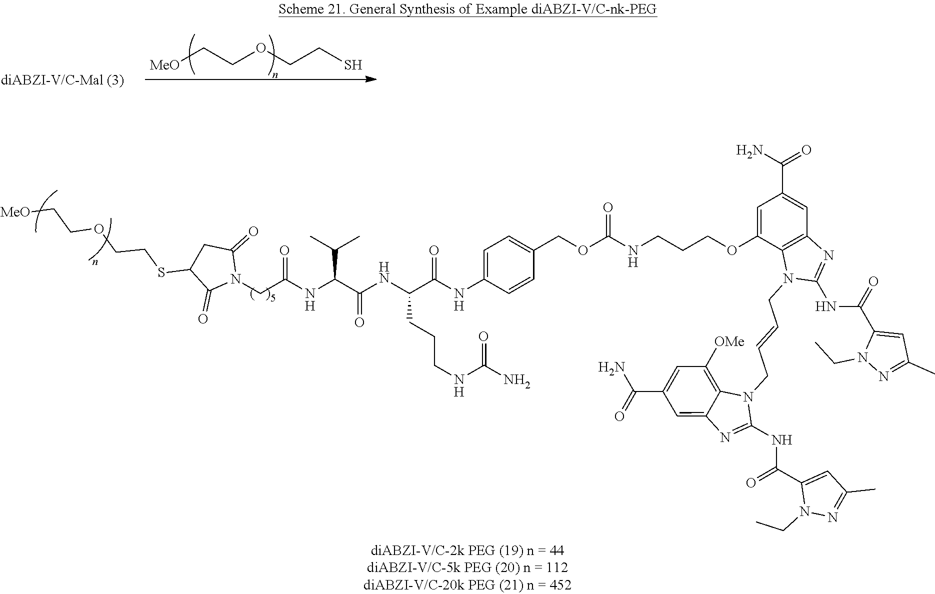

To a stirred solution of compound 3 (1.1 eq) and mPEG(nk)-SH (1.0 eq) in DMF was added solution of N-methyl morpholine (NMM, 4.0 eq) in DMF. The reaction mixture was stirred at room temperature for 2 h and purified by dialysis (3 kDa MWCO) against (1:1) DCM:MeOH (2×), acetone (2×) followed by distilled water (2×). Following dialysis, the purified compound was frozen at −80° C. for 8 h and then lyophilized for 3 days to obtain the desired purified product and characterized by ¹H NMR.

diABZI-V/C-2k PEG (19) To a stirred solution of diABZI-V/C-Mal (3) (30 mg, 21.7 μmol, 1.1 eq) and mPEG(2k)-SH (40 mg, 19.8 μmol, 1.0 eq) in DMF (2.0 mL) was added solution of N-methyl morpholine (NMM, 9 μL, 79.1 μmol, 4.0 eq) in DMF (0.5 mL). The desired purified product was lyophilized to obtain white fluffy powder (45 mg, 13.2 μmol, 67%). ¹H NMR (400 MHz, DMSO) δ 9.96 (s, 1H), 8.08 (s, 1H), 7.95 (broad s, 2H), 7.79 (d, J=8.5 Hz, 1H), 7.64 (d, J=6.7 Hz, 2H), 7.57 (d, J=8.5 Hz, 2H), 7.34-7.23 (m, 6H), 6.5 (s, 1H), 5.96 (t, J=5.0 Hz, 1H), 5.89-5.79 (m, 2H), 5.40 (s, 2H), 4.94-4.88 (m, 6H), 4.53-4.49 (m, 4H), 4.39-4.35 (m, 1H), 4.20-4.18 (m, 1H), 3.98 (t, J=5.0 Hz, 2H), 3.71 (s, 3H), 3.50 (broad s, 148H, —CH₂—CH₂— of PEGMA), 3.19-2.79 (m, 6H), 2.20-1.92 (m, 9H), 1.73-1.54 (m, 4H), 1.52-1.14 (m, 14H), 0.85 (d, J=6.7 Hz, 3H), 0.82 (d, J=6.7 Hz, 3H).

diABZI-V/C-5k PEG (20) To a stirred solution of diABZI-V/C-Mal (3) (30 mg, 21.7 μmol, 1.1 eq) and mPEG(5k)-SH (100 mg, 19.8 μmol, 1.0 eq) in DMF (2.0 mL) was added solution of N-methyl morpholine (NMM, 9 μL, 79.1 μmol, 4.0 eq) in DMF (0.5 mL). The desired purified product was lyophilized to obtain white fluffy powder (84 mg, 13.2 μmol, 66%). ¹H NMR (400 MHz, DMSO) δ 9.96 (s, 1H), 8.08 (s, 1H), 7.95 (broad s, 2H), 7.79 (d, J=8.5 Hz, 1H), 7.64 (d, J=6.7 Hz, 2H), 7.57 (d, J=8.5 Hz, 2H), 7.34-7.23 (m, 6H), 6.5 (s, 1H), 5.96 (t, J=5.0 Hz, 1H), 5.89-5.79 (m, 2H), 5.40 (s, 2H), 4.94-4.88 (m, 6H), 4.53-4.49 (m, 4H), 4.39-4.35 (m, 1H), 4.20-4.18 (m, 1H), 3.98 (t, J=5.0 Hz, 2H), 3.71 (s, 3H), 3.50 (broad s, 395H, —CH₂—CH₂— of PEGMA), 3.19-2.79 (m, 6H), 2.20-1.92 (m, 9H), 1.73-1.54 (m, 4H), 1.52-1.14 (m, 14H), 0.85 (d, J=6.7 Hz, 3H), 0.82 (d, J=6.7 Hz, 3H).

diABZI-V/C-20k PEG (21) To a stirred solution of diABZI-V/C-Mal (3) (30 mg, 21.7 μmol, 1.1 eq) and mPEG(20k)-SH (396 mg, 19.8 μmol, 1.0 eq) in DMF (2.0 mL) was added solution of N-methyl morpholine (NMM, 9 μL, 79.1 μmol, 4.0 eq) in DMF (0.5 mL). The desired purified product was lyophilized to obtain white fluffy powder (271 mg, 12.7 μmol, 64%).

### 2. Biological Evaluation of Conjugates

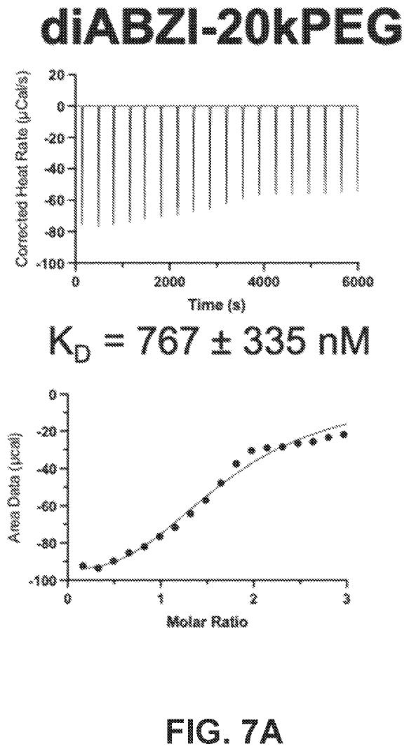

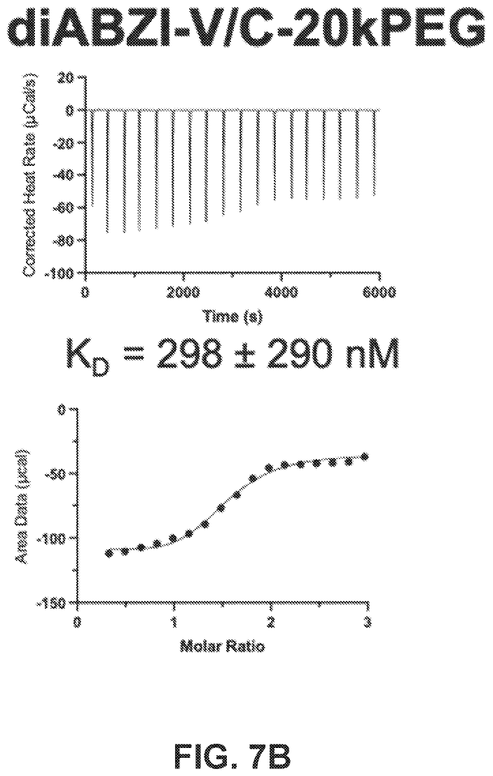

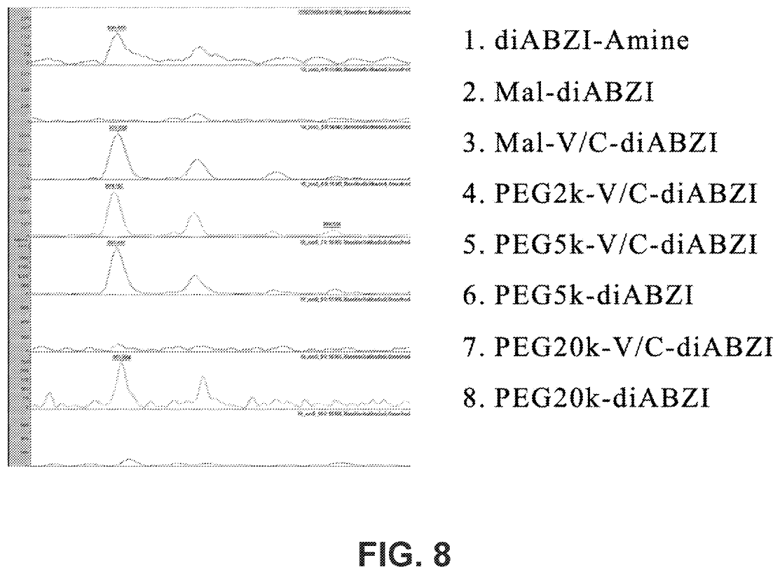

Isothermal calorimetry for STING was repeated with diABZI-20k PEG (18) and diABZI-V/C-20k PEG (21) to measure the effects of PEGylation on STING binding (FIG. 7A,7B). The binding affinities of diABZI-20k PEG (18) and diABZI-V/C-20k PEG (21) to STING were 767±335 and 298±290, respectively. There was not a difference in the K_{D} of either compound compared to the free diABZI compounds, suggest that the diABZI moiety can still access the STING binding domain even when a large polymer is attached. Additionally, the cathepsin-B assay was utilized to confirm enzyme responsiveness of these variants, once again demonstrating liberation of free drug for the compounds containing a Val-Cit linker and stability of non-cleavable variants under enzymatic conditions. (FIG. 8).

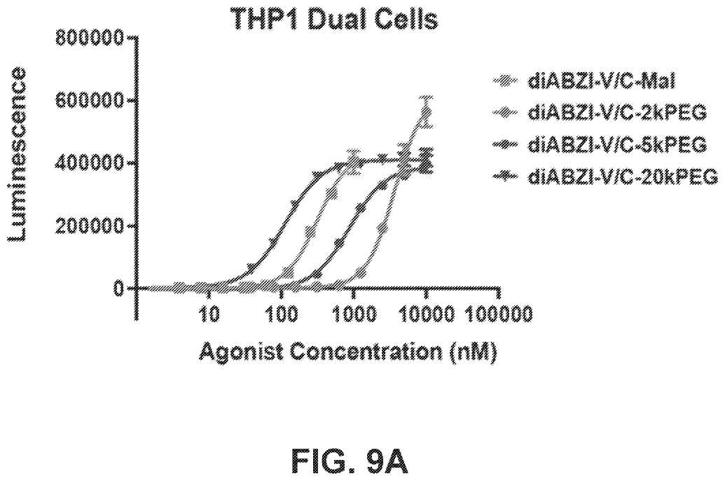

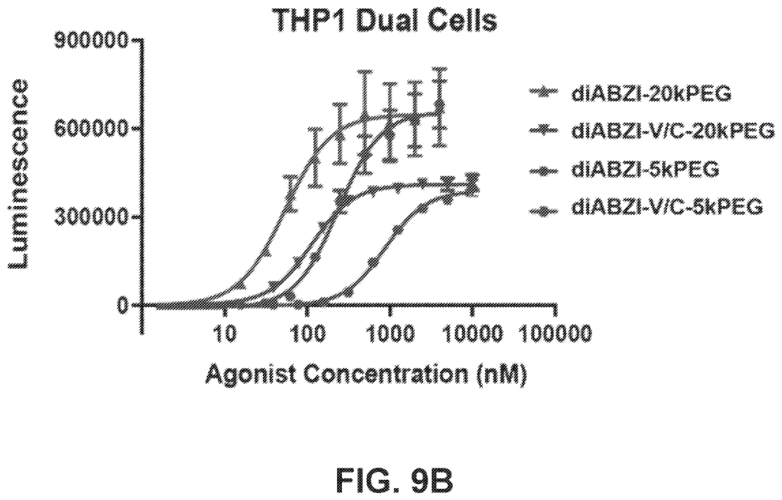

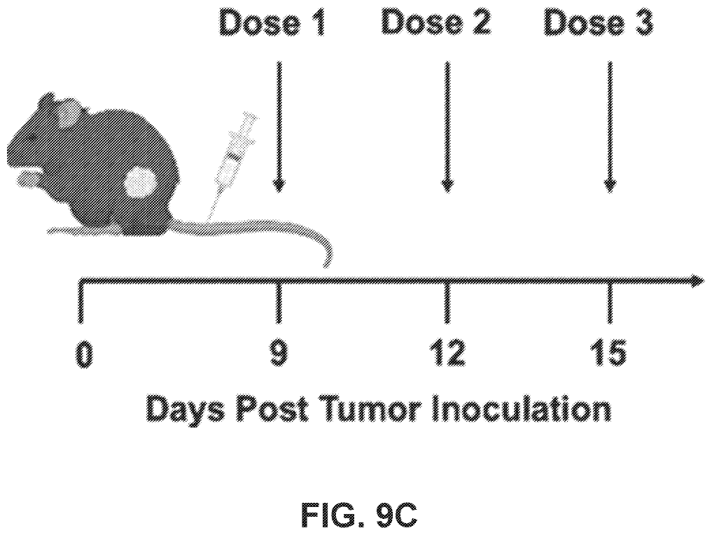

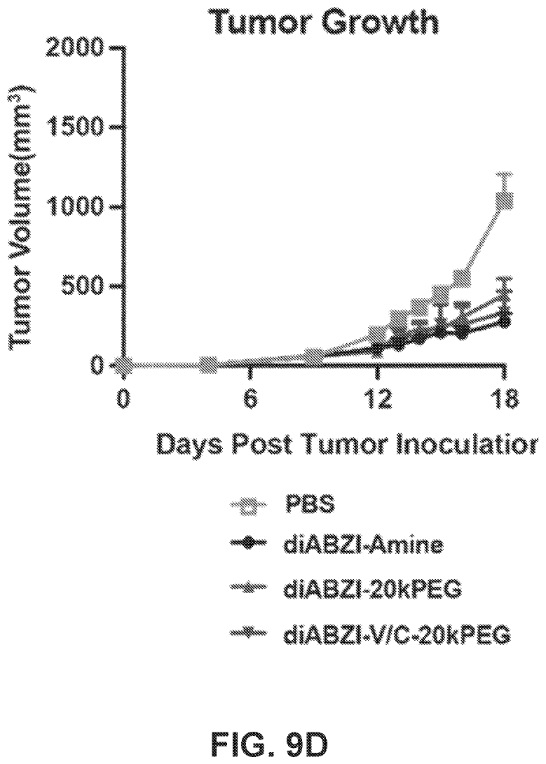

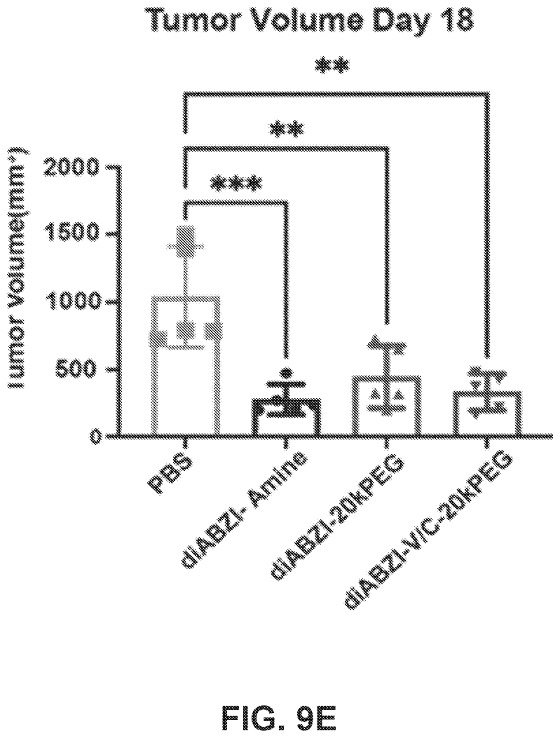

Next, TPH-1-Dual™ reporter cells were used to study the effect of diABZI PEGylation on STING activation. First, the cathepsin-cleavable variants (19), (20), and (21) were tested and all analogs were active. There was an observed trend that suggests that STING activation tended to correlate with increased molecular weight of PEG in vitro, with conjugation of 2 kDa PEG and 5 kDa PEG reducing activity compared to the parent compound (3), but conjugation of a 20 kDa PEG yielding similar or slightly enhanced activity (FIG. 9A). The activity of diABZI-PEG conjugates with 5 and 20 kDa PEGs linked via the stable or enzyme cleavable linker (i.e., compounds (17), (18), (20) and (21) were compared in vitro using the THP1-Dual™ reporter cell to further elucidate the effect of enzyme cleavage on STING activation (FIG. 9B). Surprisingly, STING activation was similar between enzyme cleavable (20) and (21) and non-cleavable (17) and (18) PEG conjugates, with non-cleavable analogs exhibiting slightly higher activity. Again, the same correlation with PEG molecular weight was observed for the non-cleavable variants, with the conjugation to 20 kDa PEG increasing activity over a 5 kDa PEG (FIG. 9A). These results potentially suggest that STING activation may be dependent on the degree of endocytosis of the macromolecular constructs, which can increase as molecular weight increases, at least in some cell types. These findings also demonstrate that diABZI can access, bind, and activate STING even when conjugated to a 20 kDa mPEG via a stable amide bond as FIG. 9B shows similar, if not better STING activation using stable rather than enzyme-responsive conjugates.

The relative in vivo activity of diABZI-20kPEG (18) and diABZI-V/C-20kPEG (21) was assessed in a B16.F10 melanoma murine tumor model, a poorly immunogenic model commonly employed in preclinical immunotherapy development, were examined. After establishing ˜50 mm³ subcutaneous B16.F10 tumors, mice were intravenously injected with either PBS (vehicle), diABZI-amine (1), diABZI-20kPEG (18), or diABZI-V/C-20kPEG (21) every 3 days for a total of 3 treatments at 0.035 umol diABZI/mouse (FIG. 9C). Consistent with in vitro studies, there were no significant differences between treatments and all inhibited tumor growth to a comparable degree (FIG. 9D). Tumor volume was measured until day 18 when the first tumor reached 1500 mm³ (FIG. 9E). Collectively, these studies demonstrate that macromolecular diABZI-PEG conjugates activate STING both in vitro and in vivo and that this is not dependent on enzyme-mediated diABZI release.

### Example 9

Synthesis and Functional Activity PEG-diABZI Conjugates with Stable and β-Glu Cleavable Linkers

### 1. Synthesis of Conjugates

### Abbreviation

PMDETA is N, N, N, N, N,-pentamethyldiethylenetriamine;
PAB is p-aminobenzylalcohol;
DMSO is dimethyl sulfoxide;
HRMS is high-resolution mas spectroscopy;
PEG is polyethylene glycol;
NMR is nuclear magnetic resonance;
atm is atmospheric pressure;
eq. or equiv. is equivalents;
rt or RT is room temperature;
hr or h is hour; and
min is minutes.

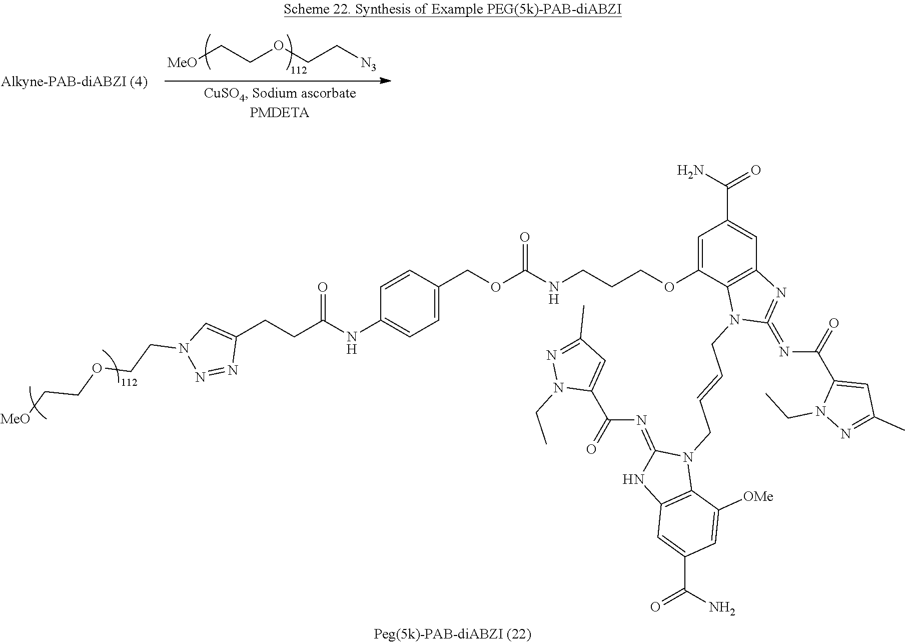

One equivalent. of 5 kDa PEG monomethoxy azide, 1.5 eq. of drug-alkyne, and 1.5 eq. of CuSO₄·5H₂O were added into the reaction vessel with 4 mL of DMF. The mixture was briefly heated and stirred to solubilize PEG then heat was removed. The mixture was degassed for 10 minutes using a stream of N₂ then under a N₂ atmosphere 15 eq of N,N,N,N,N-Pentamethyldiethylenetriamine (PMDETA) and 5 eq of sodium ascorbate was added and allowed to stir for ˜60 hours. N₂ was removed and the vessels opened to air and stirred vigorously for 6 hours to oxidize the copper to Cu(II). An excess of EDTA is added and allowed a further 3 hours to stir. 10 mL of deionized water was added, and the mixture was transferred to a 3.5 kDa membrane and dialyzed against deionized water for 24 hours. The bag was then transferred into DMSO and dialyzed against DMSO for 3 days before transferring back into deionized water overnight and lyophilizing the next day to give final solid product.

PEG(5k)-PAB-diABZI (22). ¹H NMR (400 MHz, DMSO) δ 5.82 (—CH═CH—, peaks correspond to alkene of diABZI), 3.49 (—CH₂— backbone of PEG).

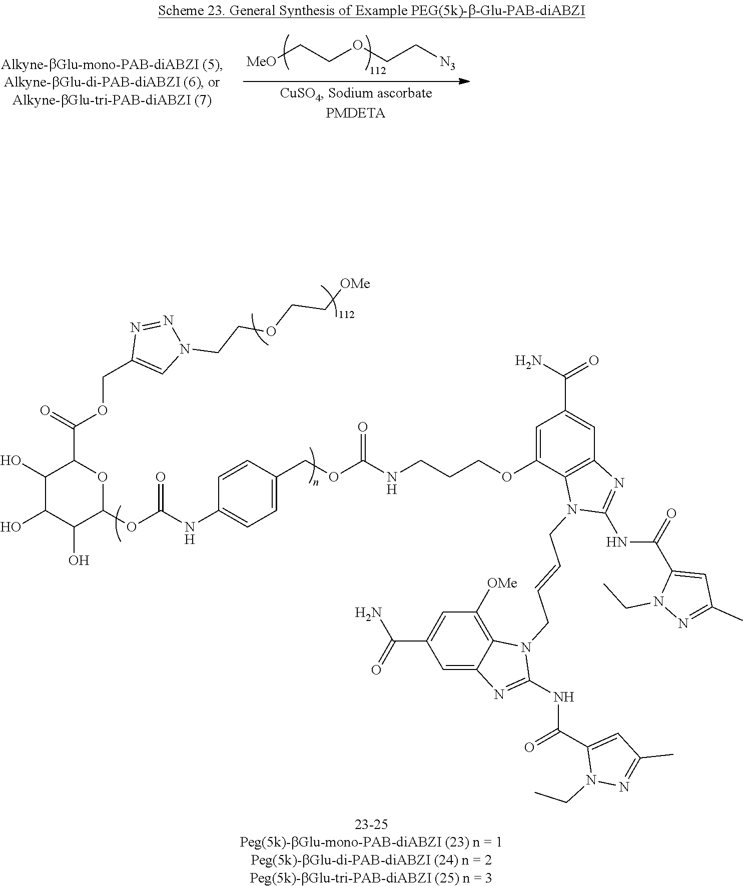

One equivalent. of 5 kDa PEG monomethoxy azide, 1.5 eq. of drug-alkyne, and 1.5 eq. of CuSO₄·5H₂O were added into the reaction vessel with 4 mL of DMF. The mixture was briefly heated and stirred to solubilize PEG then heat was removed. The mixture was degassed for 10 minutes using a stream of N₂ then under a N₂ atmosphere 15 eq of N,N,N,N,N-Pentamethyldiethylenetriamine (PMDETA) and 5 eq of sodium ascorbate was added and allowed to stir for ˜60 hours. N₂ was removed and the vessels opened to air and stirred vigorously for 6 hours to oxidize the copper to Cu(II). An excess of EDTA is added and allowed a further 3 hours to stir. 10 mL of deionized water was added, and the mixture was transferred to a 3.5 kDa membrane and dialyzed against deionized water for 24 hours. The bag was then transferred into DMSO and dialyzed against DMSO for 3 days before transferring back into deionized water overnight and lyophilizing the next day to give final solid product.

PEG(5k)-βGlu-mono-PAB-diABZI (23). ¹H NMR (400 MHz, DMSO) δ 5.82 (−CH═CH—, peaks correspond to alkene of diABZI), 3.49 (—CH₂— backbone of PEG).

PEG(5k)-βGlu-di-PAB-diABZI (24). ¹H NMR (400 MHz, DMSO) δ 5.82 (—CH═CH—, peaks correspond to alkene of diABZI), 3.49 (—CH₂— backbone of PEG).

PEG(5k)-βGlu-tri-PAB-diABZI (25). ¹H NMR (400 MHz, DMSO) δ 5.82 (—CH═CH—, peaks correspond to alkene of diABZI), 3.49 (—CH₂— backbone of PEG).

### 2. Biological Evaluation of Conjugates

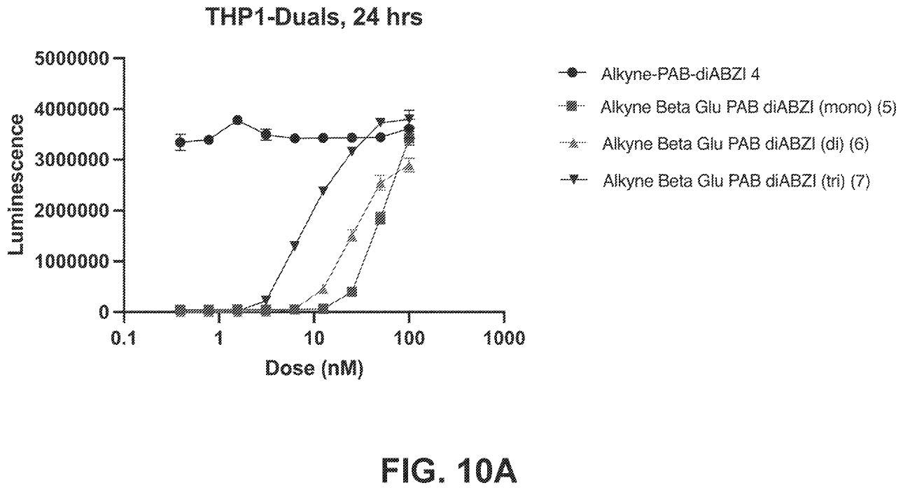

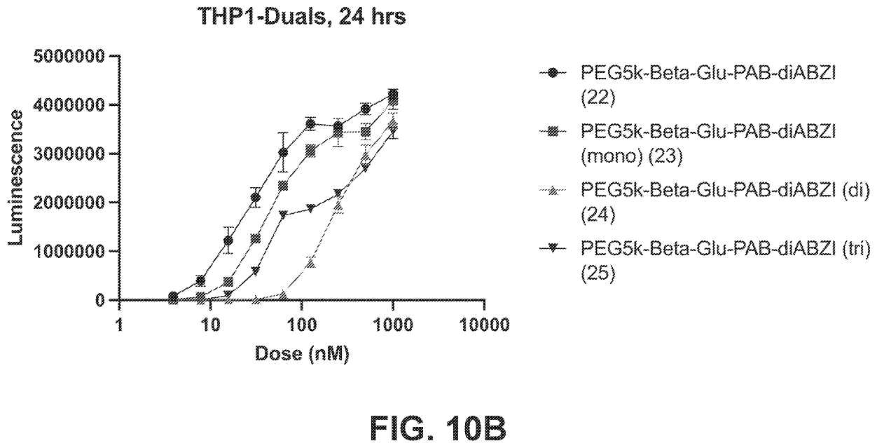

Based on the results from compounds (17-21), we wanted to determine if other PEGylated variants of diABZI with a different type of cleavable linker demonstrated similar results to the non-cleavable counterpart. A THP1 Dual™ reporter assay was completed to compare relative interferon-β production between cleavable and non-cleavable diABZI Beta-Glu variants (22-25). Consistent with previous results using cathepsin sensitive linkers, the addition of Beta-Glu responsive linker did not affect STING activation compared to the non-responsive linker, further validating that macromolecular diABZI compounds can still activate STING (FIGS. 10A and 10B).

### Example 10

### Synthesis and Functional Activity of Polymer-diABZI Conjugates via Post-Polymerization Copper-Free Click Chemistry

### 1. Synthesis of Compounds

### Abbreviation

DMA is dimethylacetamide
AzEMA is azide-ethylmethacrylate;
DMF is dimethyl formamide;
DMSO is dimethyl sulfoxide;
DBCO is dibenzocylooctyne;
HRMS is high-resolution mas spectroscopy;
NMR is nuclear magnetic resonance;
atm is atmospheric pressure;
eq. or equiv. is equivalents;
rt or RT is room temperature;
hr or h is hour; and
min is minutes.

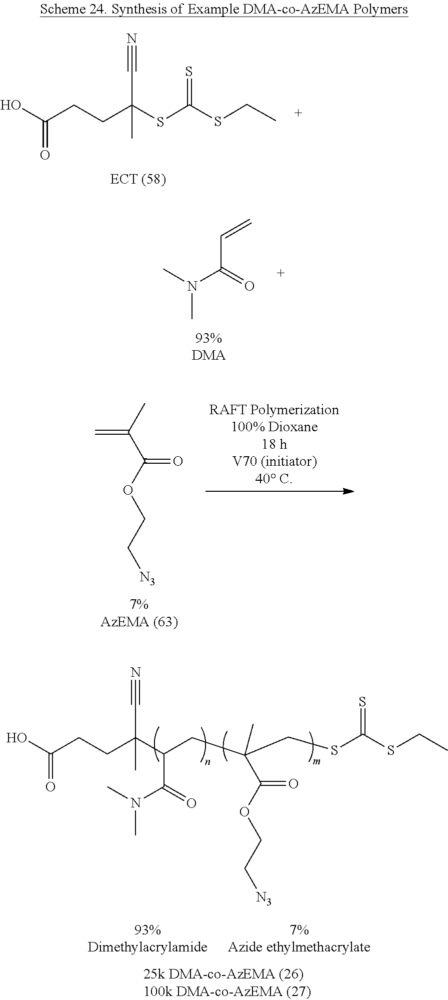

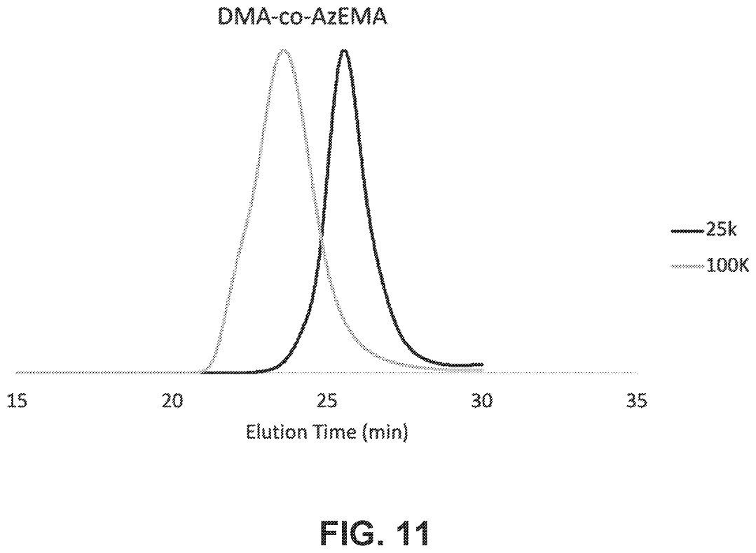

Poly(Dimethylacrylamide-co-azide-ethylmethacrylate) (poly(DMA-co-AzEMA)) were synthesized by using the chain transfer agent (ethylsulfanylthiocarbonyl)-sulfanylvpentanoic acid (ECT) and the initiator V70 with a CTA-to-initiator ratio of 5 at 40° C. for 18 hours in dioxane and purified via dialysis. We selected molecular weights of approximately 25 kDa and 100 kDa (26) and (27) since these range from below to above the renal clearance cut-off (˜40 kDa) of similar polymer systems, allowing for better differentiation of in vivo pharmacological properties such as pharmacokinetics and biodistribution. Through conversion NMR, molecular weights of 26,338 and 11,1884 Da were calculated by the percent loss of vinyl peaks post polymerization (5.5-6.5 ppm) using DMA methyl peaks as reference (3 ppm). To determine composition (DMA:AzEMA), we compared the ratio of DMA peaks (3 ppm corresponding to 6 DMA protons) and AzEMA (4.1 ppm corresponding to 2 AzEMA protons post purification. Molecular weights via GPC were determined to be 23,333 and 98,900 Da (FIG. 11).

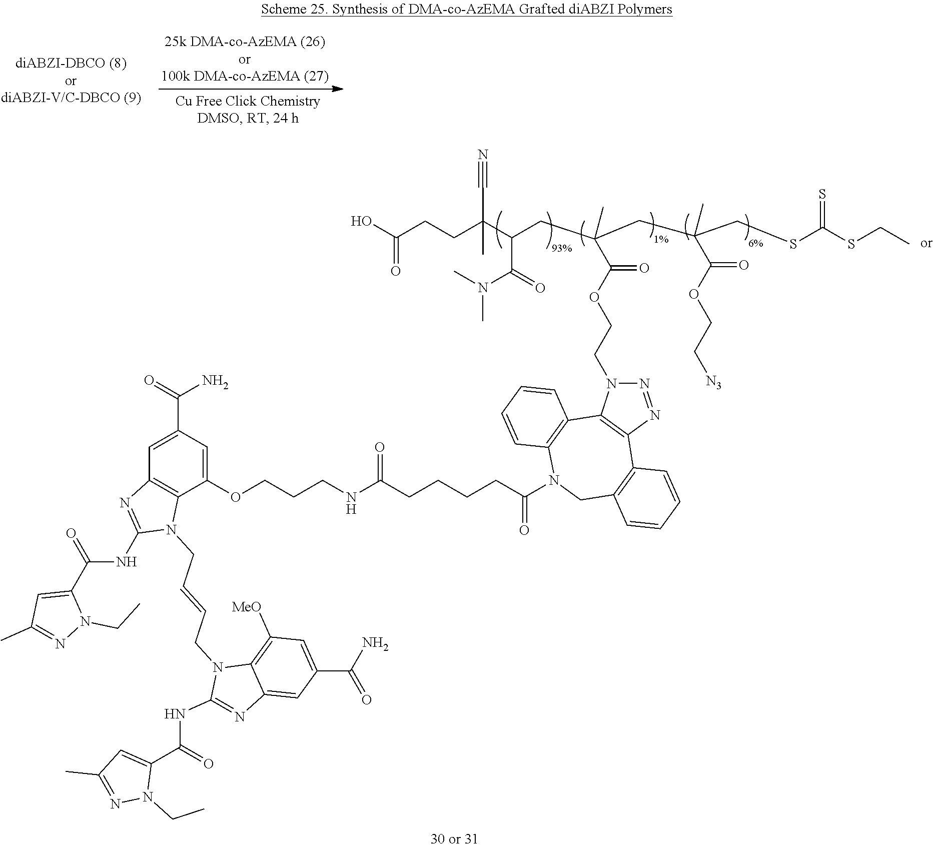

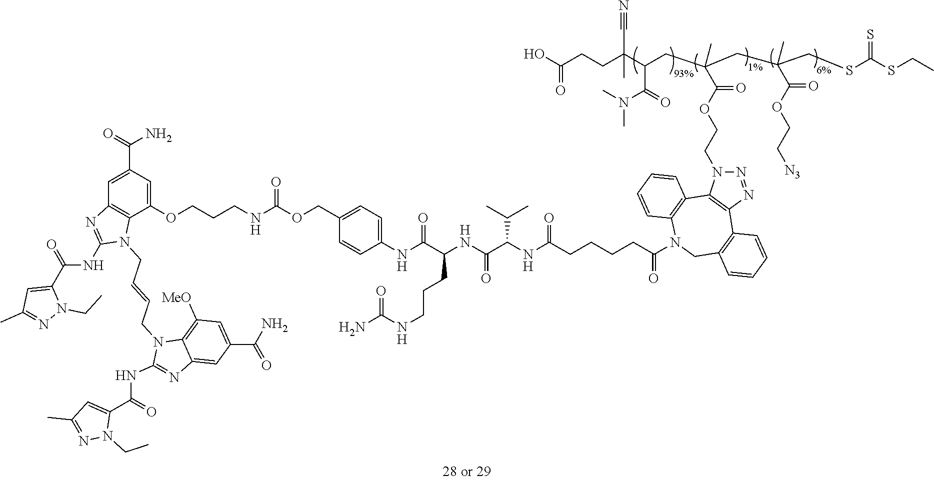

Copper-free click chemistry was used to conjugate DBCO-Cy5 dyes and DBCO functionalized diABZI variants (8 and 9) to (26) and (27). A commercial DBCO-Cy5 dye was reacted with (26) and (27) at a molar ratio of 3 dye:polymer chain. Reactions were continuously mixed for 24 hours at room temperature in 100% DMSO. Conjugates were purified via dialysis and lyophilized before conjugation to diABZI variants (8) and (9). A molar ratio of 7 diABZI:polymer was used for conjugation to the 25 kDa polymer (26) while a molar ratio of 20 diABZI:polymer was used for the 100 kDa polymer (27). The reactions were continuously mixed for 24 hours at room temperature in 100% DMSO. Conjugates were purified via DMSO dialysis in regenerated cellulose membranes, then further purified in water using centrifugal dialysis, and polymer and diABZI concentration characterized through UV-Vis spectroscopy. Reaction conditions result in approximately 1-2 Cy5 dyes for both (26) and (27) and 3-5 diABZI per 25 kDa chain (28 and 30) and 5-12 diABZI per 100 kDa chain (29 and 31).

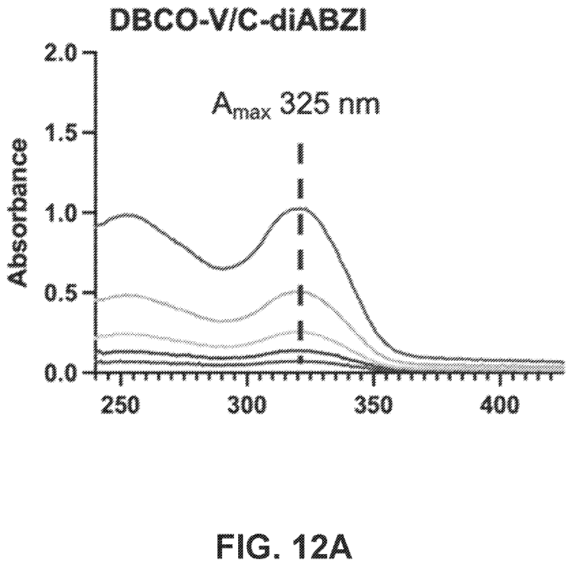

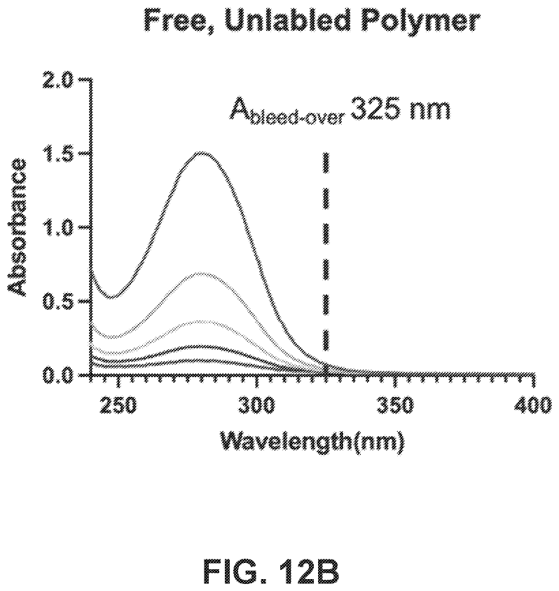

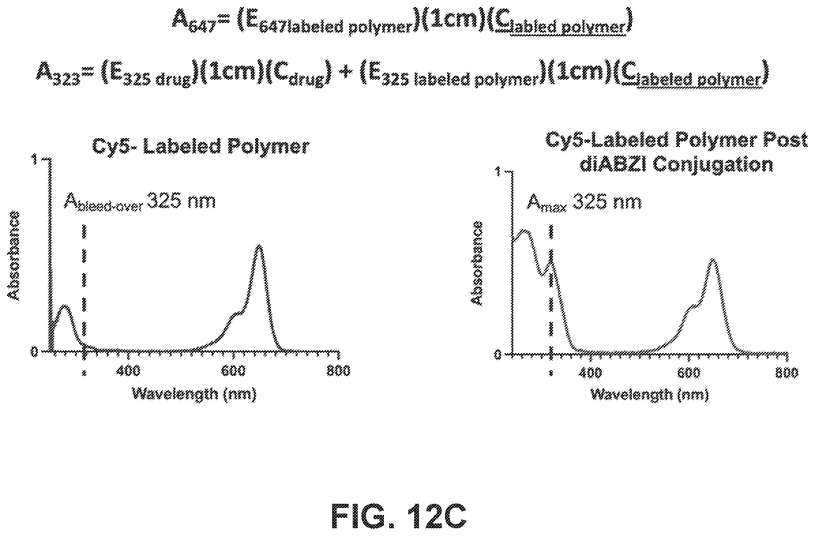

25 kDa-poly(dimethylacrylamide-co-azidethylmethacrylate)-graft-diamidobenzimidazole) (25 kDa DMA-g-V/C-diABZI) (28) Conjugates were characterized using spectrophotometric analysis thoroughly described in FIG. 12A-C, resulting in approximately 1-2 Cy5 dyes and 3-5 diABZI per chain.

100 kDa-poly(dimethylacrylamide-co-azidethylmethacrylate)-graft-diamidobenzimidazole) (100 kDa DMA-g-V/C-diABZI) (29) Conjugates were characterized using spectrophotometric analysis thoroughly described in FIG. 12A-C, resulting in approximately 1-2 Cy5 dyes and 5-12 diABZI per chain.

25 kDa-poly(dimethylacrylamide-co-azidethylmethacrylate)-graft-diamidobenzimidazole) (25 kDa DMA-g-diABZI) (30) Conjugates were characterized using spectrophotometric analysis thoroughly described in FIG. 12A-C, resulting in approximately 1-2 Cy5 dyes and 3-5 diABZI per chain.

100 kDa-poly(dimethylacrylamide-co-azidethylmethacrylate)-graft-diamidobenzimidazole)(100 kDa DMA-g-diABZI) (31) Conjugates were characterized using spectrophotometric analysis thoroughly described in FIG. 12A-C, resulting in approximately 1-2 Cy5 dyes and 5-12 diABZI per chain.

### 2. Biological Evaluation of the Conjugates

Reversible-addition-fragmentation (RAFT) polymerization was used to synthesize a series of poly(Dimethylacrylamide-co-azide-ethylmethacrylate) (poly(DMA-co-AzEMA)) polymers of various molecular weights. RAFT is advantageous due to high conversion rates, precise control of properties and monomer composition, and low polydispersity. DMA polymers with 7% AzEMA synthesized since they can react with the DBCO-functionalized diABZI compounds (8) and (9) using copper-free click chemistry for efficient drug conjugation. Having 10% or less reactive monomer was selected to provide a sufficient number of reactive groups to achieve efficient drug conjugation, while keeping the overall properties of the polymer carrier to be reflective of poly(DMA). Poly(DMA-co-AzEMA) were synthesized by using the chain transfer agent (ethylsulfanylthiocarbonyl)-sulfanylvpentanoic acid (ECT) and the initiator V70 with a CTA-to-initiator ratio of 5 at 40° C. for 18 hours in dioxane and purified via dialysis. The molecular weights of approximately 25 kDa (26) and 100 kDa (27) were synthesized since these range from below to above the renal clearance cut-off (˜40 kDa) of similar polymer systems, allowing for better differentiation of in vivo pharmacological properties such as pharmacokinetics and biodistribution. Through conversion NMR, molecular weights of 26,338 and 11,1884 Da were calculated by the percent loss of vinyl peaks post polymerization (5.5-6.5 ppm) using DMA methyl peaks as reference (3 ppm).

Copper-free click chemistry was utilized to conjugate diABZ-DBCO molecules to the azide-DMA 25 kDa and 100 kDa polymers and UV-Vis spectrophotometry was used to characterize conjugation efficiency. The diABZI molecules have a signature absorbance peak at 325 nm, allowing for concentration to be determined spectrophotometrically (FIG. 12A-B). However, the CTA on the polymers also contributes slightly to the absorbance at this wavelength, which could result in an overestimation of drug concentration. To compensate for this, 1-2 Cy5-DBCO dyes were conjugated to the polymer chains prior to diABZI conjugation through copper-free click chemistry, allowing for a distinctive absorbance peak from which to calculate polymer concentration and subtract its contribution to A₃₂₅, thereby allowing the concentration of diABZI in solution to be determined (FIG. 12C). Cy5 dye conjugation also allows for fluorescent imaging and quantification polymer concentration in blood or other biological fluid following in vivo administration. A molar ratio of 7 diABZI:polymer was used for conjugation to the 25 kDa polymer while a molar ratio of 20 diABZI:polymer was used for the 100 kDa polymer. The reactions were continuously mixed for 24 hours at room temperature in 100% DMSO. Conjugates were purified via DMSO dialysis in regenerated cellulose membranes, then further purified in water using centrifugal dialysis, and polymer and diABZI concentration characterized through UV-Vis spectroscopy. Reaction conditions result in approximately 3-5 diABZI per 25 kDa chain and 5-12 diABZI per 100 kDa chain.

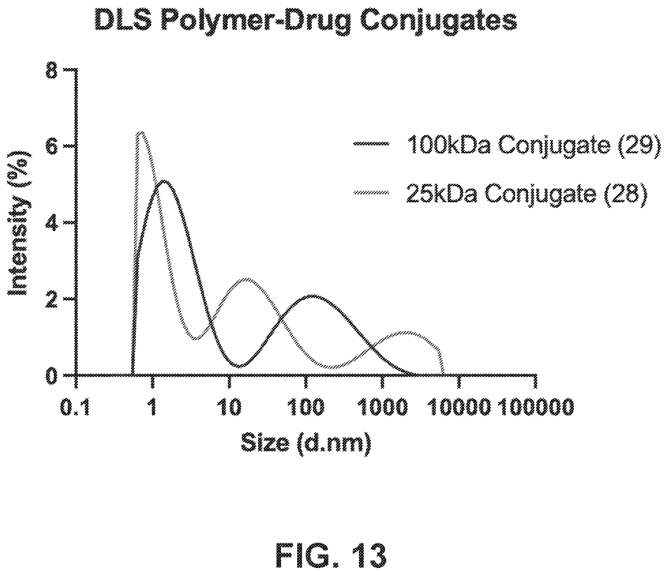

We assessed DMA-diABZI conjugate size using dynamic light scattering (DLS) which did not indicate the formation particulate structures (FIG. 13).

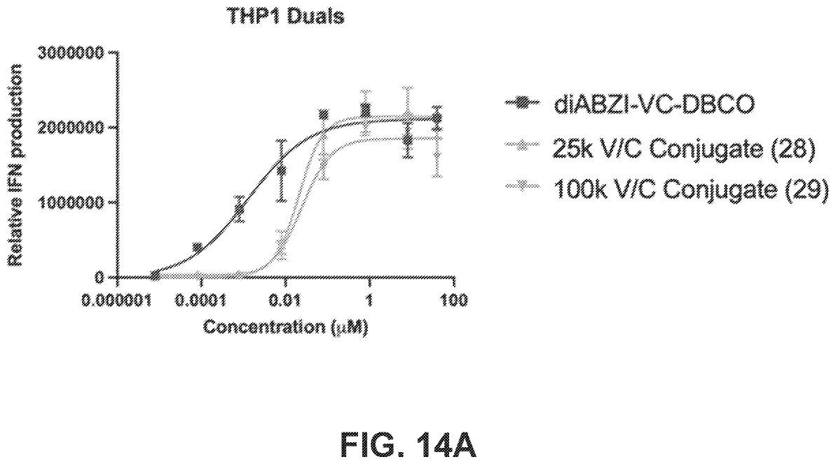

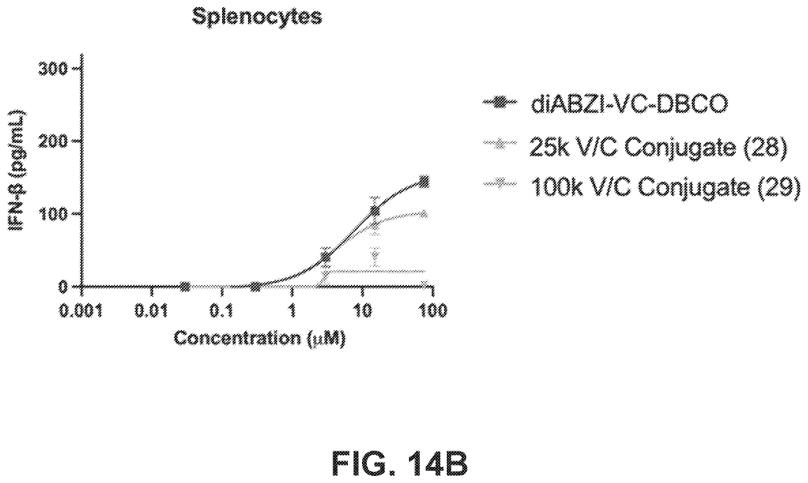

In vitro characterization of DMA-c-diABZI conjugates for STING pathway activation was performed using both a THP-1-Dual™ type-I interferon (IFN) reporter cell line and primary murine splenocytes by measuring interferon-β secretion into supernatant by ELISA as described elsewhere. (FIG. 14A, B). While conjugation of the diABZI to polymer chains with a V/C linker reduced in vitro activity, this is not unexpected as large hydrophilic macromolecules constructs may take longer to enter cells and/or access STING.

Based on findings described above that stable (i.e., amide linked) PEG-diABZI conjugates are able to activate STING, 25 kDa and 100 kDa DMA-c-diABZI conjugates were prepared using a non-cleavable linker to form stable (30) and (31). Stable variants were prepared with the same reaction and characterization methods as described for the cleavable conjugates.

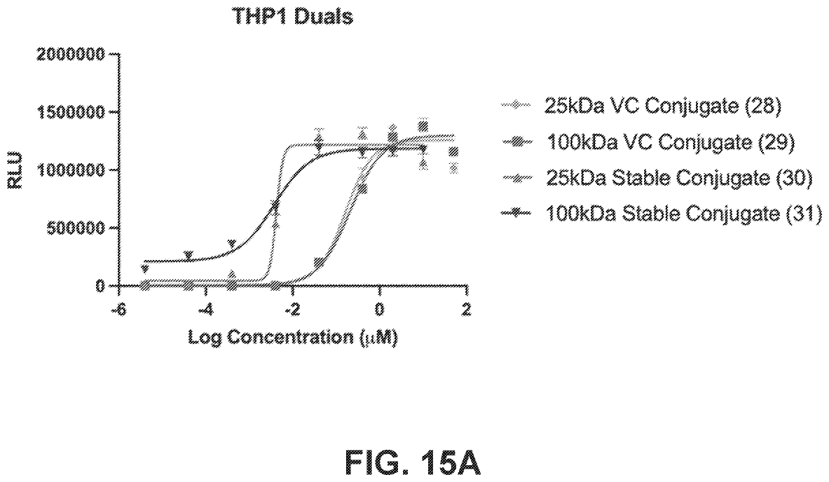

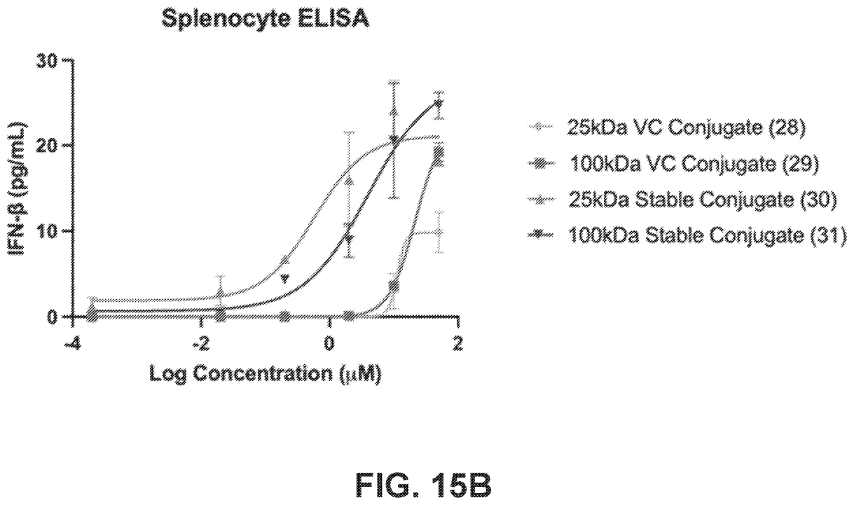

The activity of stable and enzyme cleavable DMA-c-diABZI conjugates in both THP-1 Dual™ IFN-I reporter cells and primary murine splenocytes was performed as (FIG. 15A, B). Surprisingly, and similar to the findings with PEG-diABZI conjugates, the stable conjugates tended to have more activity for STING activation, further suggesting that diABZI release from polymer carriers may not be a requirement for STING pathway activation.

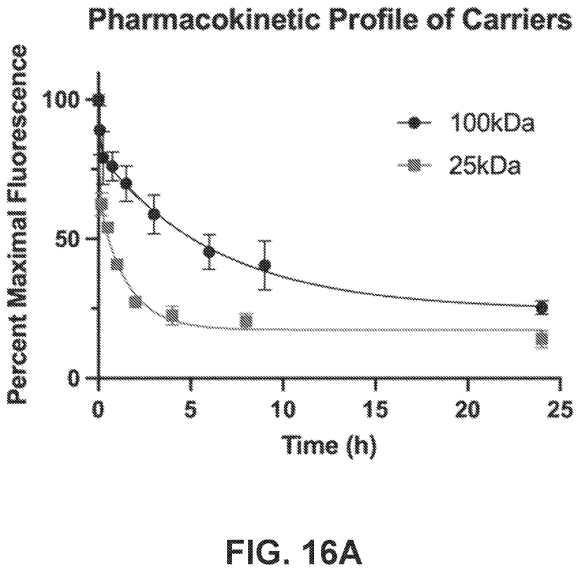

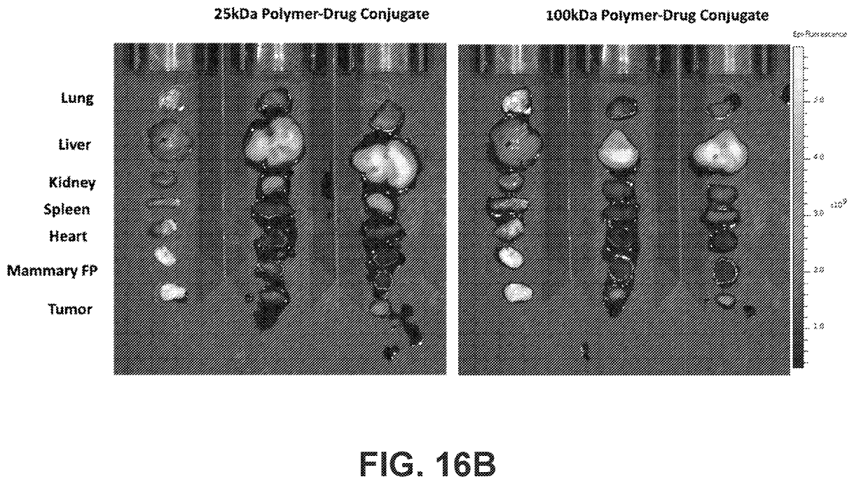

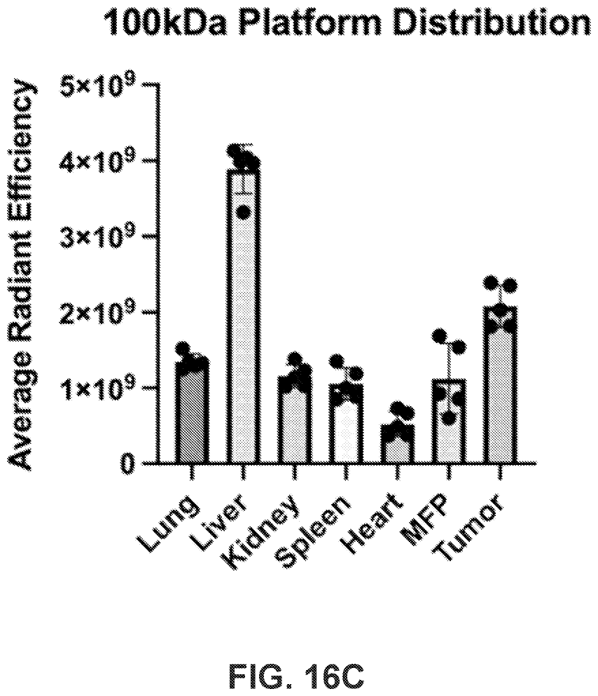

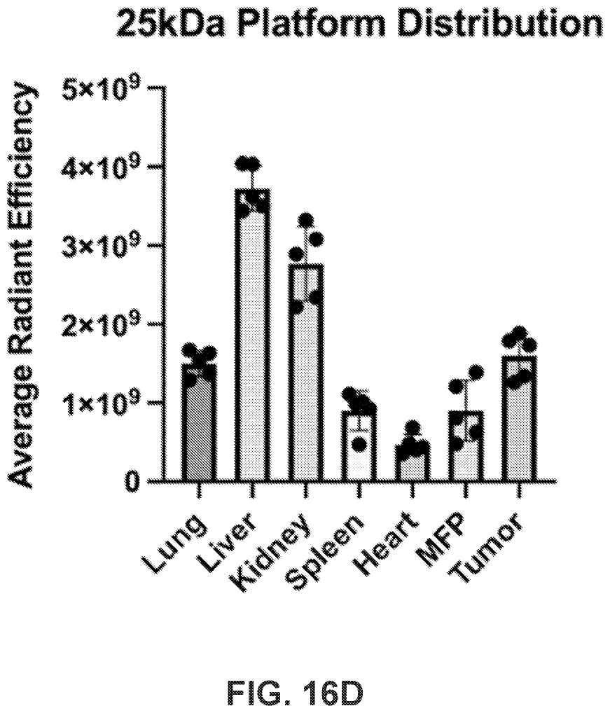

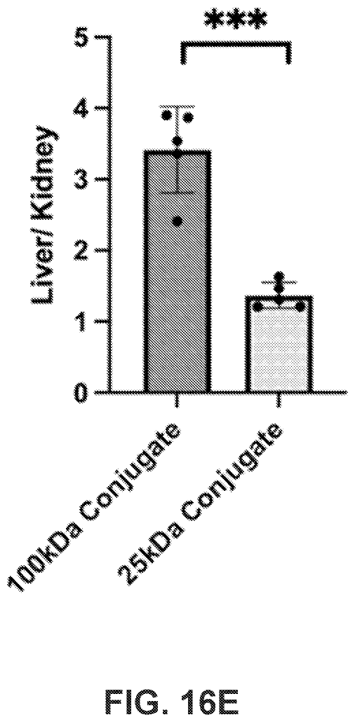

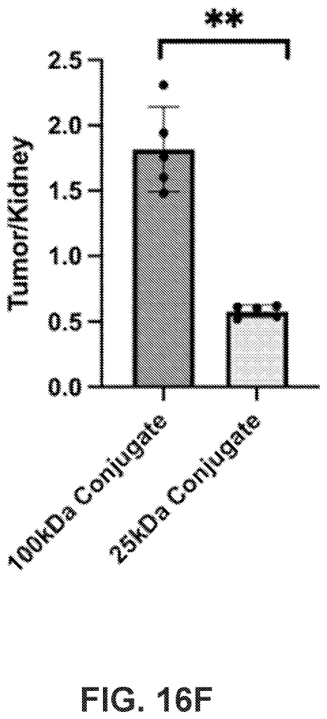

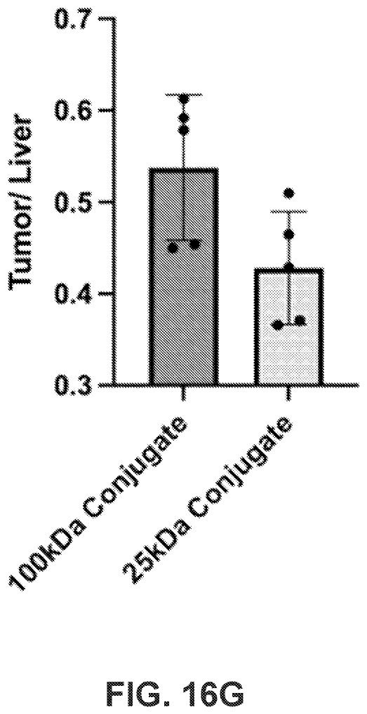

The pharmacokinetics (PK) and biodistribution of 25 kDa and 100 kDa DMA-g-diABZI conjugates (28) and (29) were prepared using cleavable V/C linkers and labeled with ˜1-2 Cy5 dyes per chain. To determine PK properties of conjugates, healthy mice were intravenously injected with either the 25 kDa or 100 kDa platform (n=5) at a diABZI concentration of approximately 0.012 μmol/mouse and blood was sampled for quantification of polymer concentration using fluorescence spectroscopy over 24 hours. Mice treated with vehicle (PBS) mice (n=2) were also used to subtract background autofluorescence of the blood. A serum half-life of approximately 1.07 hours and 4.4 hours were determined for the 25 kDa platform and the 100 kDa platform, respectively (FIG. 16A). These data demonstrate that the higher molecular weight polymer demonstrated prolonged circulation due to mitigated renal clearance and that the PK profile of diABZI can be modulated using polymeric carriers.

A biodistribution study was also performed to understand organ accumulation of DMA-g-V/C-diABZI polymer-drug conjugates (28) and (29) (FIG. 16B). Mice growing ˜50 mm³ E0771 murine breast tumors in a mammary fat pad were treated systemically with either the 25 kDa or 100 kDa DMA-g-V/C-diABZI conjugates corresponding to approximately 0.012 μmol diABZI/mouse (n=5) or PBS (n=2) and organs were resected after 24 hours for IVIS® imaging. Compound (29) showed accumulation mainly in the liver and tumor, with a definitive shift from renal clearance (FIG. 16C, E-G). Compound (28) demonstrated accumulation in the kidney, tumor, and liver, indicating the use of renal clearance mechanisms (FIG. 16D-G). Both systems demonstrated increased tumor accumulation compared to the contralateral mammary fat pad, as a distinction between cancerous vs non-cancerous tissue.

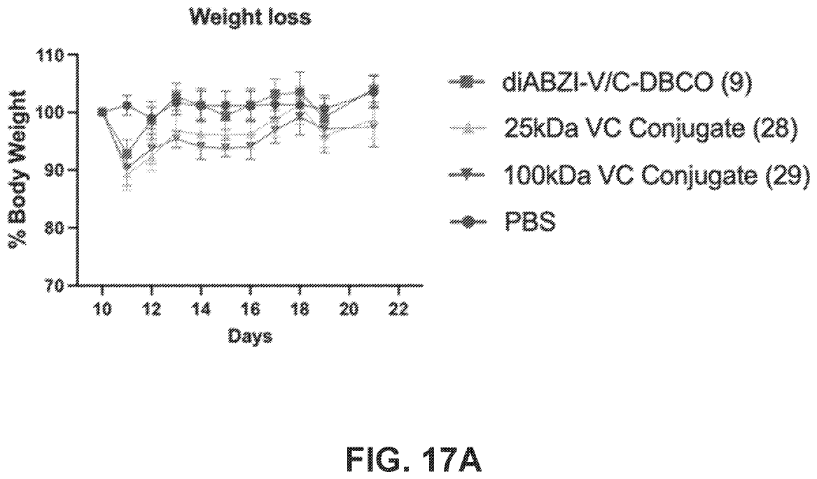

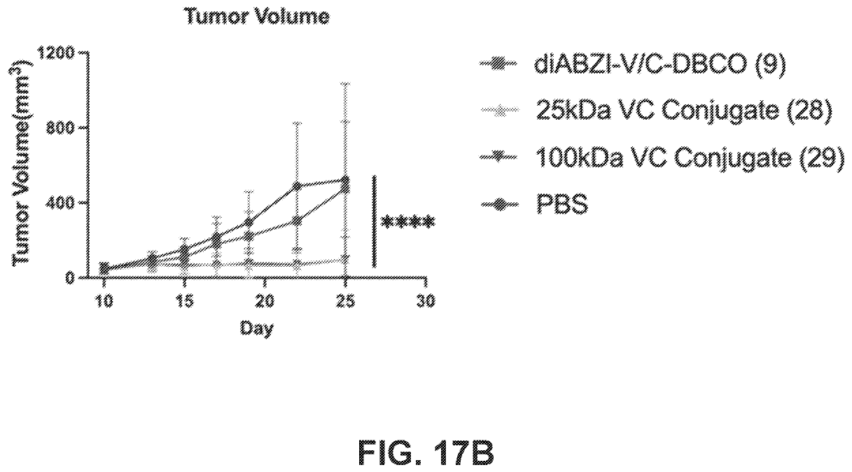

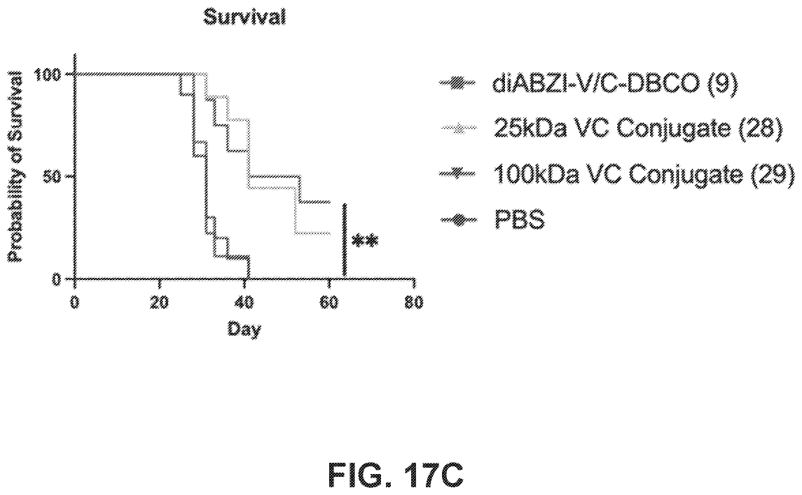

To test the therapeutic efficacy of the DMA-g-V/C-diABZI conjugates, mice were inoculated with mammary fat pad E0771 murine breast tumors, which were grown to approximately 45 mm³. They were systemically injected with either 25 kDa or 100 kDa DMA-g-V/C-diABZI or free diABZI-V/C-DBCO corresponding to approximately 0.009 μmol drug/mouse every 4 days for 3 total treatments. Weight loss was measured to understand transient toxicity of the drugs, which all remained within the acceptable range for innate immune agonists (FIG. 17A). Both conjugates demonstrated significantly improved therapeutic response compared to the free diABZI-V/C-DBCO and vehicle (PBS) with approximately 20% of 25 kDa and 37.5% of 100 kDa treated mice with complete tumor regression (FIG. 17B). Survival of the mice treated with (28) and (29) was longer than the control and non-polymer conjugated compound (FIG. 17C).

### Example 11

Synthesis and Functional Activity Polymer diABZI Conjugates Using diABZI Functionalized Chain Transfer

### 1. Synthesis of Conjugates

### Abbreviations

DMA is dimethylacetamide
CTA is chain transfer agent;
ECT is 4-cyano-4-(ethylsulfanylthiocarbonyl) sulfanylvpentanoic acid;
AzPMAm is N-(3-azidopropyl)methacrylamide;
PDMA is poly(N,N dimethylacrylamide);
PEGMA is polyethylene glycol methacrylate;
DMA is dimethyl acrylamide;
PDSMA is pyridyl disulfide ethyl methacrylate;
AIBN is azoisoisobutyronitrile;
DMF is dimethyl formamide;
DMSO is dimethyl sulfoxide;
HRMS is high-resolution mas spectroscopy;
NMR is nuclear magnetic resonance;
atm is atmospheric pressure;
eq. or equiv. is equivalents;
rt or RT is room temperature;
hr or h is hour; and
min is minutes.

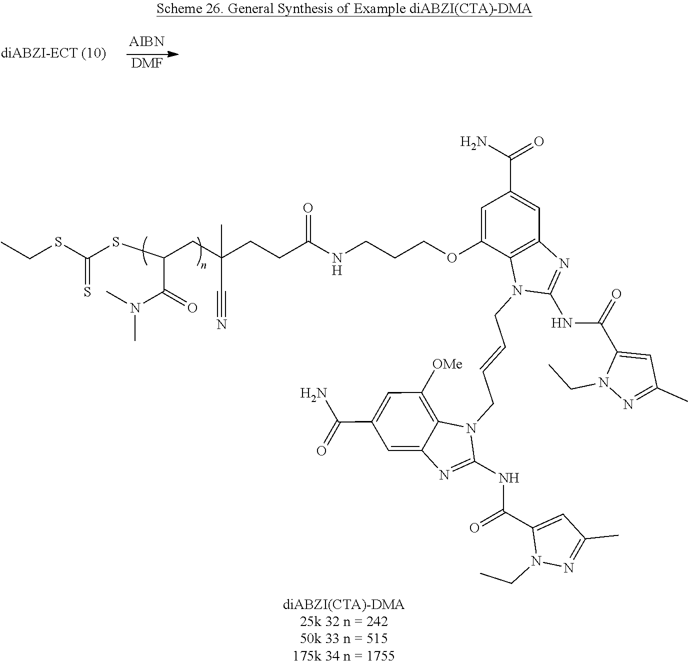

Reversible addition-fragmentation chain transfer (RAFT) was used to synthesize three analogs of polymers with distinct molar masses (25k, 50k, 175k). For synthesis dimethyl acrylamide (DMA) monomer was filtered over activated alumina and allowed to react under inert atmosphere in DMF (30 wt % monomer) at 70° C. for 24 h in an oil bath. The initial monomer ([M]ₒ) to diABZI-CTA compound (10) ([CTA]ₒ) to initiator ([I]ₒ) ratio was n:1:0.2. The resultant desired polymers DMA(nk)-diABZI-CTA was isolated by dialysis against pure acetone (2×), pure deionized water (2×). Following dialysis, the purified compound was frozen at −80° C. for 8 h and then lyophilized for 3 days.

diABZI(CTA)-DMA-25k (32) The initial monomer ([M]ₒ) to diABZI-CTA compound (10) ([CTA]ₒ) to initiator ([I]ₒ) ratio was 242:1:0.2. The desired polymer (32) was obtained after dialysis as white solid (255 mg, 84%) which was further characterized by ¹H NMR. ¹H NMR (400 MHz, DMSO) δ 3.06-2.64 (dimethyl group PDMA), 2.62-2.11 (—CH— backbone of PDMA), 1.67-1.00 (—CH₂— backbone of PDMA).

diABZI(CTA)-DMA-50k (33) The initial monomer ([M]ₒ) to diABZI-CTA compound (10) ([CTA]ₒ) to initiator ([I]ₒ) ratio was 515:1:0.2. The desired polymer (33) was obtained after dialysis as white solid (365 mg, 72%) which was further characterized by ¹H NMR. ¹H NMR (400 MHz, DMSO) δ 3.06-2.64 (dimethyl group PDMA), 2.62-2.11 (—CH— backbone of PDMA), 1.67-1.00 (—CH₂— backbone of PDMA).

diABZI(CTA)-DMA-175k (34) The initial monomer ([M]ₒ) to diABZI-CTA compound (10) ([CTA]ₒ) to initiator ([I]ₒ) ratio was 1755:1:0.2. The desired polymer (34) was obtained after dialysis as white solid (663 mg, 65%) which was further characterized by ¹H NMR. ¹H NMR (400 MHz, DMSO) δ 3.06-2.64 (dimethyl group PDMA), 2.62-2.11 (—CH— backbone of PDMA), 1.67-1.00 (—CH₂— backbone of PDMA).

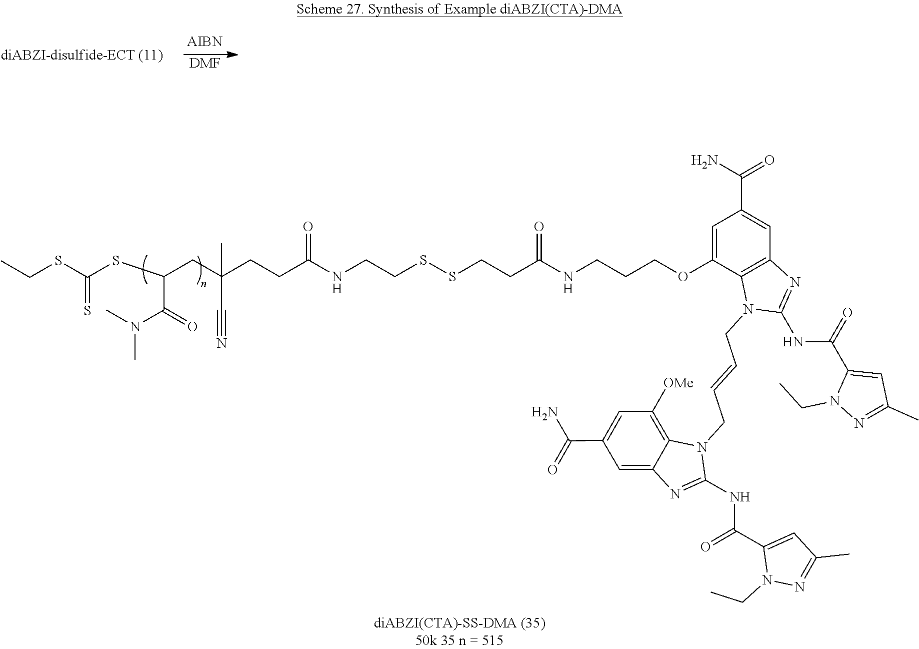

Reversible addition-fragmentation chain transfer (RAFT) was used to synthesize polymer of molar mass 50k. For synthesis dimethyl acrylamide (DMA) monomer was filtered over activated alumina and allowed to react under inert atmosphere in DMF (30 wt % monomer) at 70° C. for 24 h in an oil bath. The initial monomer ([M]ₒ) to diABZI-SS-CTA compound (11) ([CTA]ₒ) to initiator ([I]ₒ) ratio was n:1:0.2. The resultant desired polymers DMA(nk)-diABZI-SS-CTA was isolated by dialysis against pure acetone (2×), pure deionized water (2×). Following dialysis, the purified compound was frozen at −80° C. for 8 h and then lyophilized for 3 days.

diABZI(CTA)-SS-poly-DMA (50k) (35) The initial monomer ([M]ₒ) to diABZI-CTA compound (11) ([CTA]ₒ) to initiator ([I]ₒ) ratio was 493:1:0.2. The desired polymer (35) was obtained after dialysis as white solid (157 mg, 80%) which was further characterized by ¹H NMR. ¹HNMR (400 MHz, DMSO) δ 3.06-2.64 (dimethyl group PDMA), 2.62-2.11 (—CH— backbone of PDMA), 1.67-1.00 (—CH₂— backbone of PDMA).

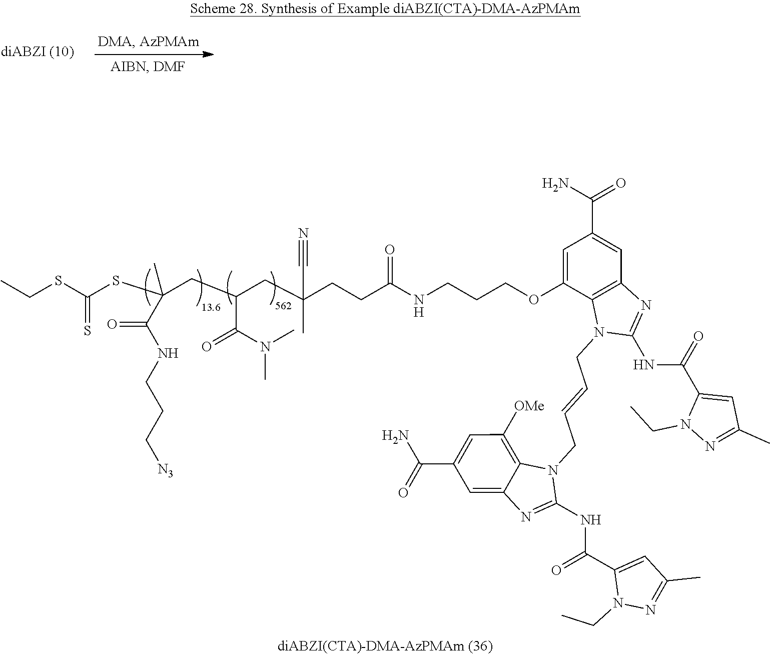

Reversible addition-fragmentation chain transfer (RAFT) was used to synthesize polymer of molar mass 50 kDa. For synthesis, dimethyl acrylamide (DMA) monomer, filtered over activated alumina, and azidopropyl methacrylamide (AzPMAm) were allowed to react under inert atmosphere in DMF (30 wt % monomer) at 70° C. for 24 h in an oil bath. The initial DMA monomer ([DMA]ₒ) to AzPMAm monomer ([AzPMAm]ₒ) to diABZI-ECT compound (10) ([CTA]ₒ) to initiator ([I]ₒ) ratio was 562:13.6:1:0.2. The resultant desired polymer, 50 kDa diABZI-DMA-co-AzPMAm (36), was isolated by dialysis against pure acetone (2×) and then pure deionized water (2×). Following dialysis, the purified compound was frozen at −80° C. for 8 h and then lyophilized for 3 days.

diABZI(CTA)-DMA-AzPMAm (36) ¹H NMR (400 MHz, DMSO) δ 3.06-2.64 (dimethyl group PDMA), 2.62-2.11 (—CH— backbone of PDMA), 1.67-1.00 (—CH₂— backbone of PDMA).

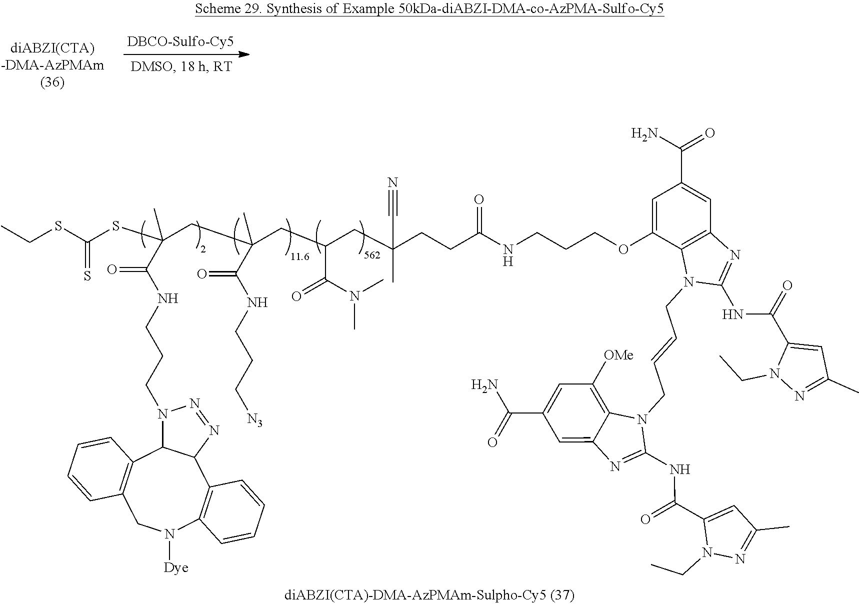

50 kDa diABZI-DMA-co-AzPMAm (36) was reacted with dibenzocyclooctyne (DBCO)-functionalized sulfo-Cyanine5 (DBCO-sCy5) molecules in DMF (30 wt % monomer) at RT for 18 h on a magnetic stir plate. The initial DBCO-sCy5 compound ([DBCO-sCy5]ₒ) to 50 kDa diABZI-DMA-co-AzPMAm ([Polymer]ₒ) ratio was 1:0.4. The resultant desired polymer-sCy5 conjugate, diABZI-DMA-co-AzPMA-Sulfo-Cy5 (37), was isolated by dialysis against pure 1:1 dichloromethane:methanol (2×), pure acetone (2×), and then pure deionized water (2×). Following dialysis, the purified compound was frozen at −80° C. for 8 h and then lyophilized for 3 days.

The desired polymer-sCy5 conjugate (37) was obtained after dialysis as blue solid which was characterized for sCy5 loading via UV-Vis using the literature extinction coefficient for sCy5.

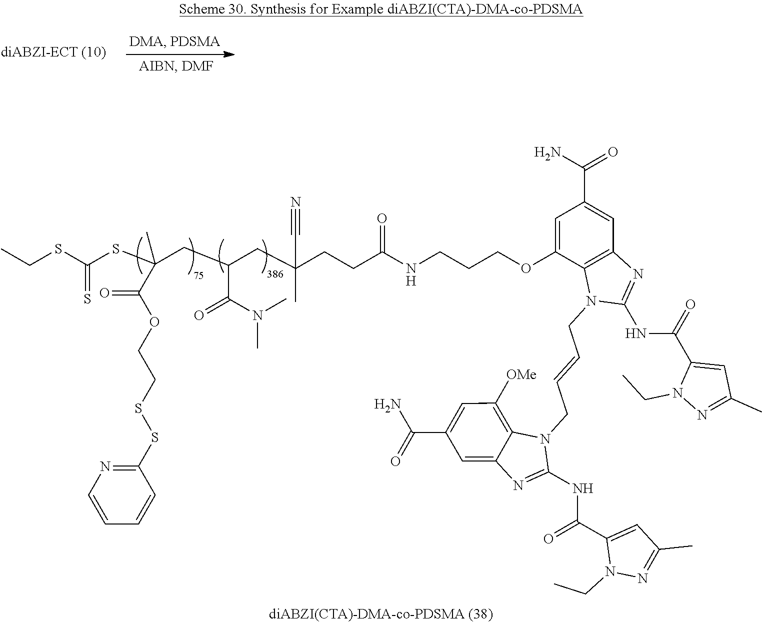

Reversible addition-fragmentation chain transfer (RAFT) was used to synthesize polymer of molar mass 50 kDa. For synthesis, dimethyl acrylamide (DMA) monomer, filtered over activated alumina, and pyridyl disulfide ethyl methacrylate (PDSMA) were allowed to react under inert atmosphere in DMF (30 wt % monomer) at 70° C. for 24 h in an oil bath. The initial DMA monomer ([DMA]ₒ) to PDSMA monomer ([PDSMA]ₒ) to diABZI-ECT compound (10) ([CTA]ₒ) to initiator ([I]ₒ) ratio was 425:75:1:0.2. The resultant desired polymer, 50 kDa diABZI-DMA-co-PDSMA (38), was isolated by dialysis against pure acetone (2×) and then pure deionized water (2×). Following dialysis, the purified compound was frozen at −80° C. for 8 h and then lyophilized for 3 days.

diABZI(CTA)-DMA-co-PDSMA (38) The initial DMA monomer ([DMA]ₒ) to PDSMA monomer ([PDSMA]ₒ) to diABZI-ECT compound (10) ([CTA]ₒ) to initiator ([I]ₒ) ratio was 425:75:1:0.2. The desired polymer (38) was obtained after dialysis as white solid (330 mg, 36%) which was further characterized by ¹H NMR. ¹H NMR (400 MHz, DMSO) δ 8.44 (—CH of PDSMA), 7.78 (—CH of PDSMA), 7.22 (—CH of PDSMA), 3.06-2.64 (dimethyl group PDMA), 2.62-2.11 (—CH— backbone of PDMA), 1.67-1.00 (—CH₂— backbone of PDMA).

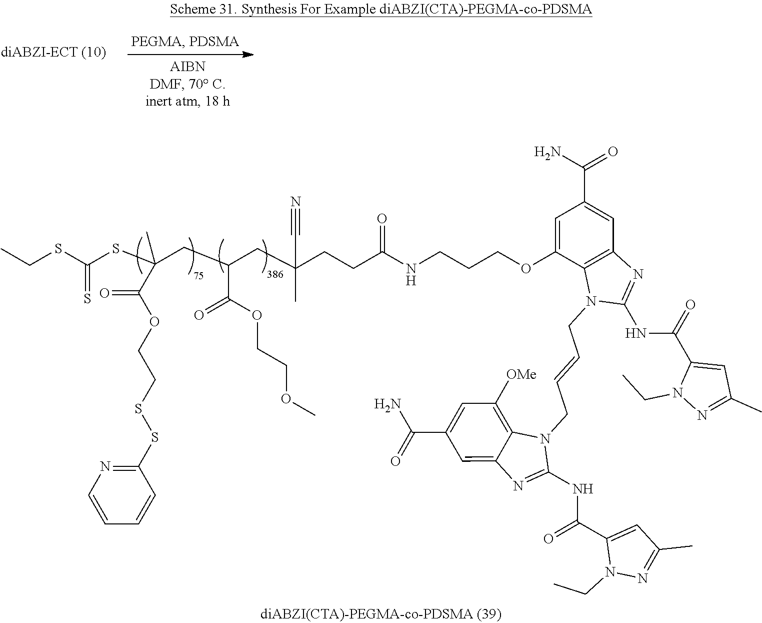

Reversible addition-fragmentation chain transfer (RAFT) was used to synthesize polymer of molar mass 50 kDa. For synthesis, polyethylene glycol methacrylate (PEGMA) monomer, filtered over activated alumina, and pyridyl disulfide ethyl methacrylate (PDSMA) were allowed to react under inert atmosphere in DMF (30 wt % monomer) at 70° C. for 24 h in an oil bath. The initial PEGMA monomer ([PEGMA]ₒ) to PDSMA monomer ([PDSMA]ₒ) to diABZI-ECT compound (10) ([CTA]ₒ) to initiator ([I]ₒ) ratio was 150:75:1:0.2. The resultant desired polymer, 50 kDa diABZI-PEGMA-co-PDSMA (39), was isolated by dialysis against pure acetone (2×) and then pure deionized water (2×). Following dialysis, the purified compound was frozen at −80° C. for 8 h and then lyophilized for 3 days.

diABZI(CTA)-PEGMA-co-PDSMA (39). The initial PEGMA monomer ([PEGMA]ₒ) to PDSMA monomer ([PDSMA]ₒ) to diABZI-ECT compound (10) ([CTA]ₒ) to initiator ([I]ₒ) ratio was 150:75:1:0.2. The desired polymer (39) was obtained after dialysis as white solid (573 mg, 64%) which was further characterized by ¹H NMR. δ 8.47 (—CH of PDSMA), 7.77 (—CH of PDSMA), 7.25 (—CH of PDSMA), 3.50 (—CH2— of PEGMA).

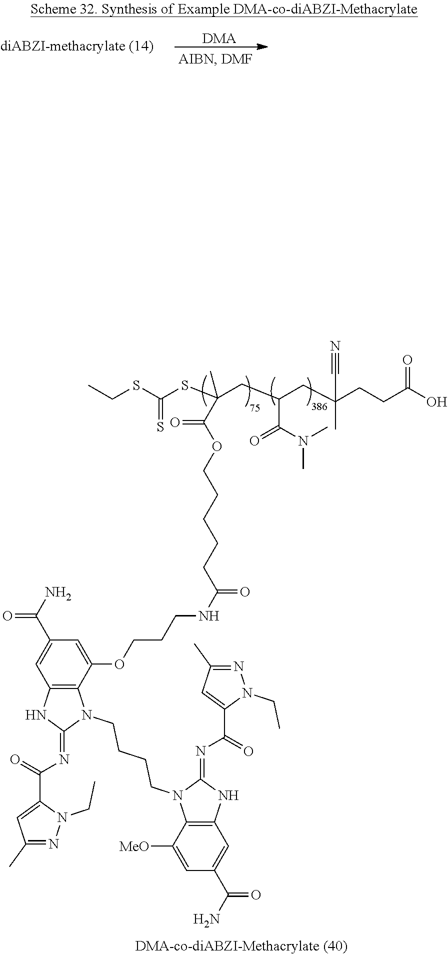

Reversible addition-fragmentation chain transfer (RAFT) was used to synthesize polymer of molar mass 40 kDa. For synthesis, dimethyl acrylamide (DMA) monomer, filtered over activated alumina, and diABZI-Methacrylate were allowed to react under inert atmosphere in DMF (30 wt % monomer) at 70° C. for 24 h in an oil bath. The initial DMA monomer ([DMA]ₒ) to diABZI-Methacrylate monomer ([diABZI-Methacrylate]ₒ) to ([CTA]ₒ) to initiator ([I]ₒ) ratio was 386:75:1:0.2. The resultant desired polymer, 50 kDa DMA-co-diABZI-Methacrylate (40), was isolated by dialysis against pure 1:1 dichloromethane:methanol, pure acetone (2×), and then pure deionized water (2×). Following dialysis, the purified compound was frozen at −80° C. for 8 h and then lyophilized for 3 days.

DMA-co-diABZI The initial DMA monomer ([DMA]ₒ) to diABZI-Methacrylate monomer ([diABZI-Methacrylate]ₒ) to ([CTA]ₒ) to initiator ([I]ₒ) ratio was 386:75:1:0.2. The desired polymer (40) was obtained after dialysis as white solid (10 mg, 18%).

### 2. Biological Activity of the Conjugates

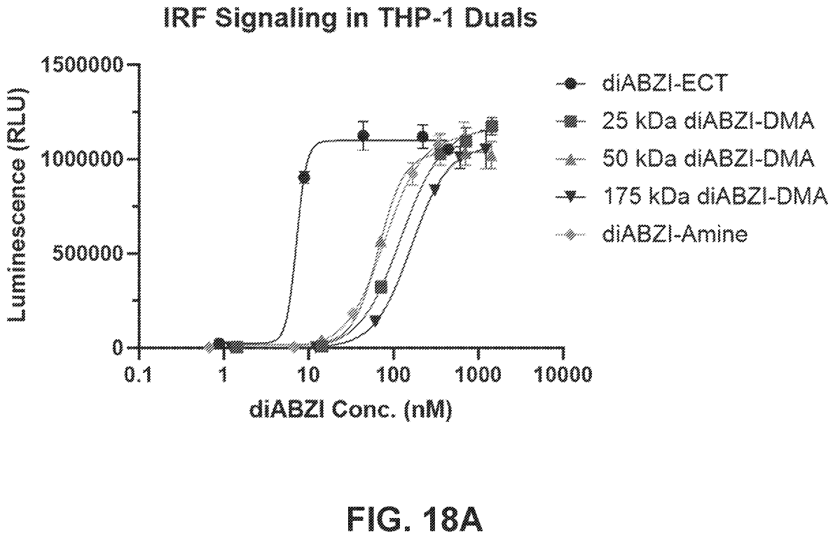

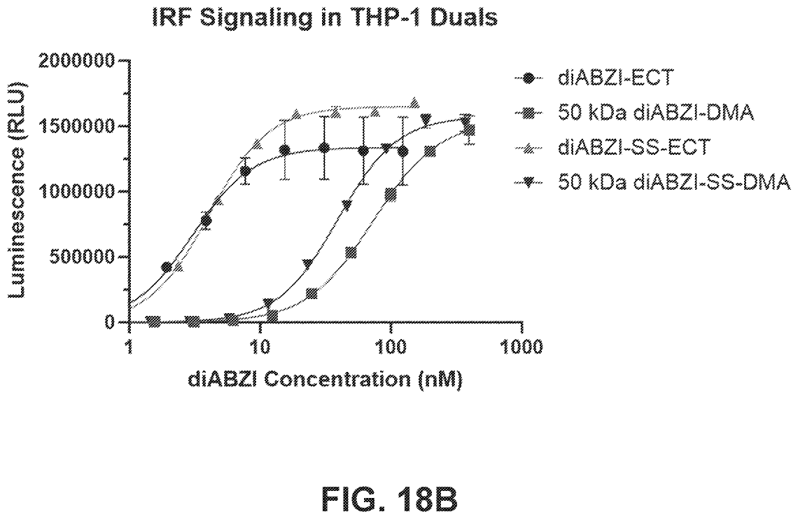

The capacity of diABZI-DMA and diABZI-SS-ECT conjugates to activate STING in THP-1 Dual™ reporter cells (FIG. 18A, 18B). Consistent with our findings with stable diABZI-PEG conjugates, all diABZI-DMA constructs activated STING to a similar degree with EC₅₀ values in the nanomolar range, regardless of polyDMA molecular weight (Table 2). Moreover, it was found that diABZI-SS-DMA constructs activated STING in a similar range as the stably linked constructs. It was also found that the diABZI-ECT (10) and diABZI-SS-ECT were significantly more potent than diABZI-amine (1), likely owing to differences in membrane permeability that were also reflected in the increased activity of compound (2) relative to the amine-modified parent molecule. Therefore, while chain extension of the diABZI-ECT and diABZI-SS-ECT with polyDMA reduces activity, these studies further demonstrate that diABZI stably and reversibly conjugated to bulky, water-soluble macromolecules can activate STING signaling and, remarkably, with a similar potency to the amine-functionalized parent compound diABZI-amine (1) despite a ˜150× difference in molecular weight in stably linked constructs.

TABLE 2
Functional activity (EC₅₀ values) of diABZI-Amine
(1), diABZI-ECT (10), 25 kDa diABZI-DMA (32), 50
kDa diABZI-DMA (33), and 175 kDa diABZI-DMA (34).

| Compound | EC₅₀ value (nM) |
|---|---|
| diABZI-Amine (1) | 78.6 |
| diABZI-ECT (10) | 7.21 |
| 25 kDa diABZI-DMA (32) | 123 |
| 50 kDa diABZI-DMA (33) | 67.3 |
| 175 kDa diABZI-DMA (34) | 163 |

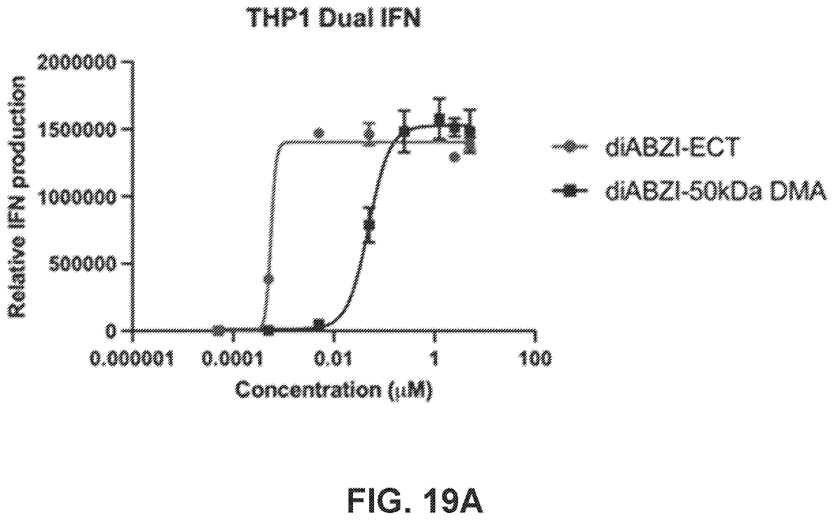

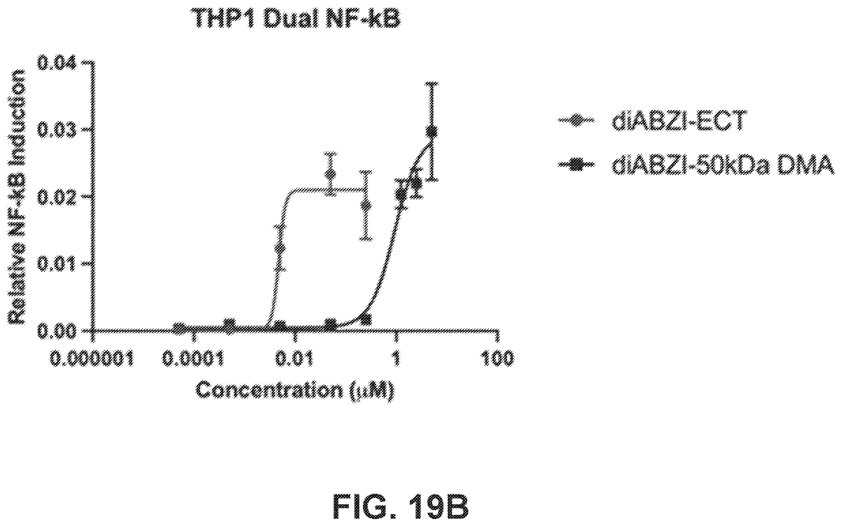

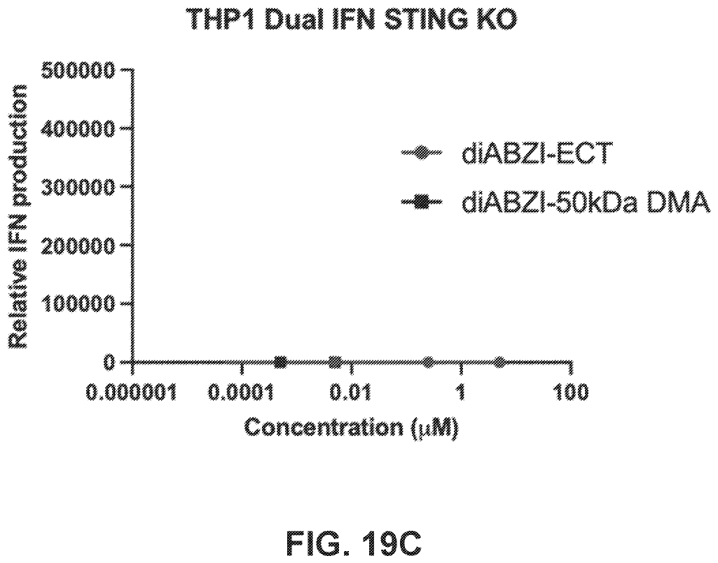

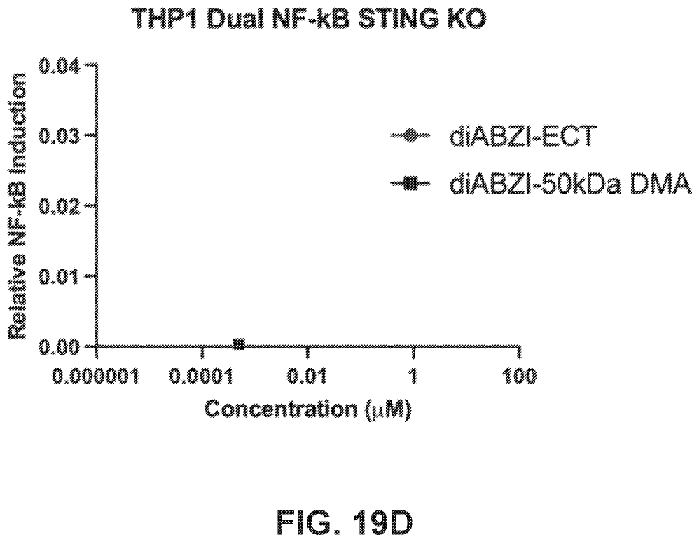

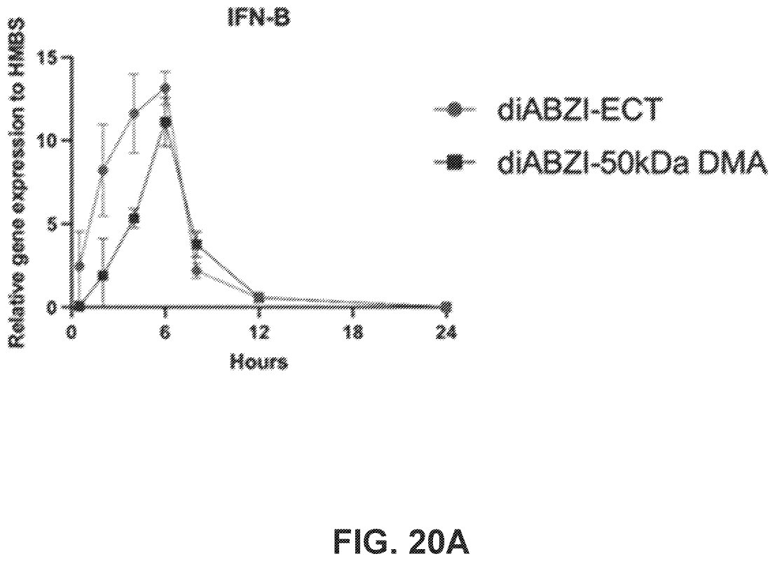

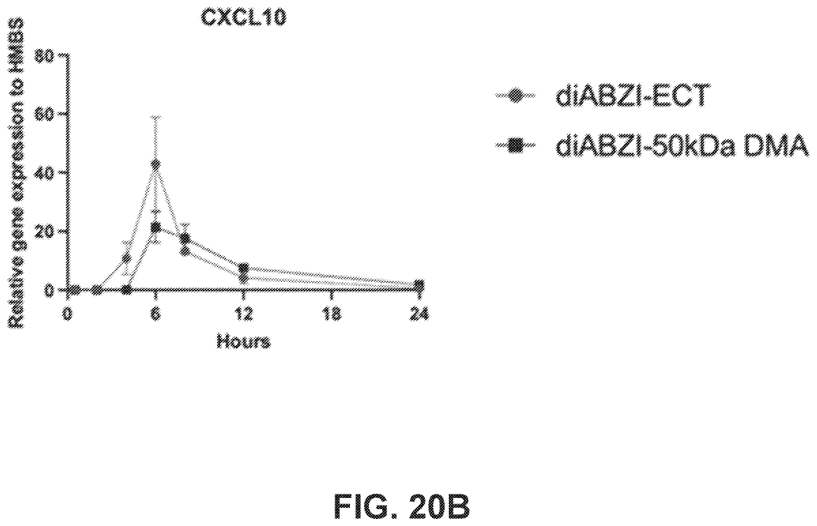

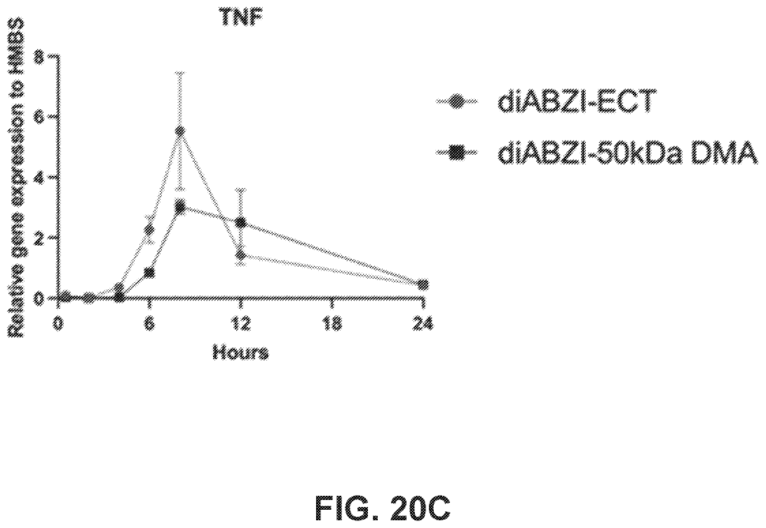

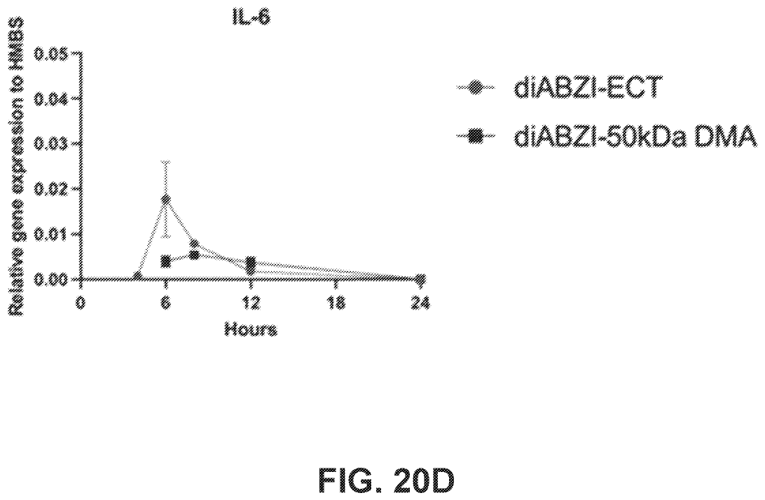

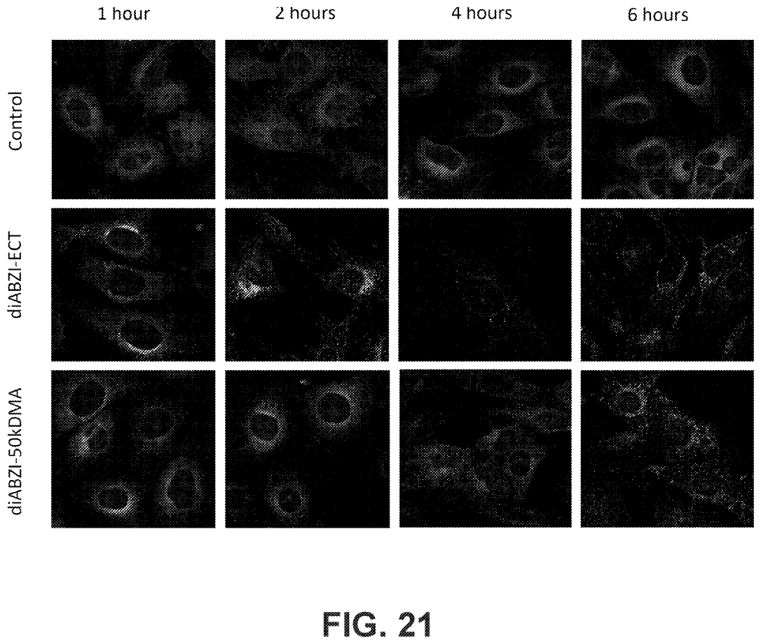

THP-1™ Duals were used to measure both IRF and NF-kb pathway activation comparing diABZI-ECT (10) and 50 kDa poly(DMA)-diABZI (33) since differences in IRF and NF-kb responses can indicate differences in where STING is activated, i.e. within the endo-lysosome or on the ER. Results indicated that both signaling pathways are engaged by the small molecule and macromolecular diABZI in a similar fashion (FIG. 19A-B). It was validated that conjugate activated IRF and NF-κβ responses in a STING-dependent manner by demonstrating a lack of activity of compounds in a STING knockout THP1 Dual™ cell line (FIG. 19C-D). PCR was performed to study differences in the kinetics of STING activation between the two molecules, which appear to initiate STING-mediated gene expression on similar time scales despite large differences in molecular weight (FIG. 20A-D). Expression of type-I interferon induction appears to occur earlier than expression of NF-kb-driven genes, but both pathways were once again activated by both molecules. To further understand STING dynamics, a murine embryonic fibroblast cell line engineered to contain green fluorescent protein (GFP)-expressing STING was treated with 2.5 mM diABZI (either diABZI-ECT or 50 kDa DMA-diABZI) and GFP was imaged over several hours using confocal microscopy (FIG. 21). ER-bound STING activation visually appears to occur in the same timeframe as reported through PCR, indicated by the formation of STING-puncta and eventual degradation of the protein. It is not surprising that 50 kDa DMA-diABZI has delayed STING activation since cellular uptake is dependent on endocytosis, which is a slower process than membrane diffusion of a small molecule.

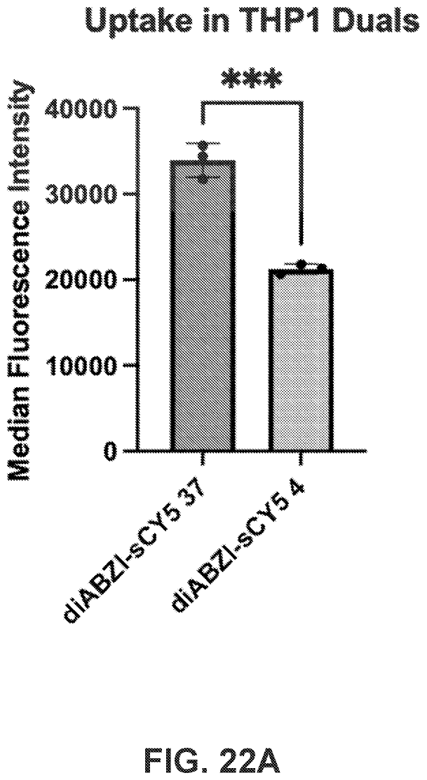

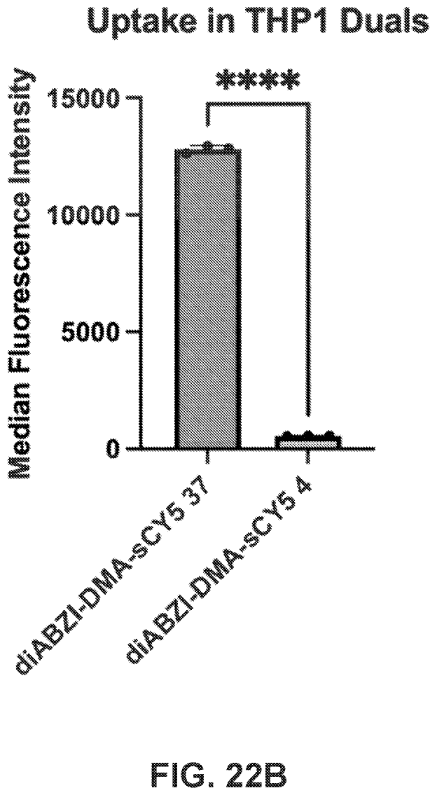

To confirm the dependence of cellular uptake on endocytosis, a RAFT polymerization was used to synthesize a fluorescent version of 50 kDa DMA-diABZI (36) in which a small amount of azide propyl methacrylamide (2%) was co-polymerized to allow for covalent conjugation to DBCO-Sulfo-Cy5 dye, yielding compound (37), which had 2 Sulfo-Cy5 molecules per chain. The cellular uptake of sCy5-labeled diABZI (16) and sCy5-labeled DMA-diABZI (37) at both 37° C. and 4° C. using flow cytometry (FIG. 22A-B). The uptake of diABZI-DMA was nearly completely abrogated at 4° C., indicating a dependence on endocysosis for cellular uptake. Therefore, DMA-diABZI appears to cells via endocytosis where it can activate STING signaling within ˜2 hr.

As demonstrated with conjugation of a DBCO-dye to azide groups on diABZI-p(DMA-co-APMAm), the integration of reactive monomer into the DMA block allows for covalent conjugation of other agents. This concept was further expanded using the diABZI-ECT to polymerize poly(DMA-co-PDSMA) (38) and poly(PEGMA-co-PDSMA) (39) of 50 kDa where pendant PDS groups enable conjugation to thiol-containing molecules via disulfide exchange reactions.

### Example 12

### Biological Methods

hSTING Binding Studies. Recombinant hSTING was synthesized in Escherichia coli (New England Biolabs, T7 Shuffle Express line) and purified by affinity chromatography. Buffer exchange was performed prior to ITC (pH 7.5: PBST, 150 mM NaCl, 3 mM EDTA, 0.05% Tween 20), using Amicon Ultra 4 mL centrifugal filters (Millipore, Etobicoke, Canada). ITC experiments were performed on a TA Instruments Affinity ITC instrument. 24 total injections were performed using the following instrument settings: cell temperature 25° C., reference power 10 μCal/second, initial delay 240 seconds, stirring speed 75 rpm, feedback mode/gain high, and injection volume 2 μL for 10 seconds spaced at 120 second intervals with a filter period of 10 seconds. hSTING was set in the cell at a concentration of 10 μM and a volume of 350 μL. Agonists were prepared at a stock concentration of 20 mM in DMSO and diluted using pH 7.5 PBST to 150 μM for titration by the syringe (120 μL). Data were analyzed using TA Instruments NanoAnalyze Software.

Enzyme Cleavability Studies. Recombinant Mouse Cathepsin B (RND Systems) was prepared at 50 μM in MES buffer (pH 5.0) upon opening and kept in −80° C. conditions when not in use. To activate the enzyme, Cathepsin B was diluted to 0.2 μM in MES buffer (pH 5.0) containing 1 mM EDTA and 2 mM DTT and placed at 37° C. for 15 minutes. After activation, Cathepsin B was combined with 50 μM of substrate at 37° C. for 37 hours at a total volume of 100 μL. Activity was determined by observing molecular weight shifts in the substrate using matrix assisted laser desorption and ionization mass spectrometry (MALDI-MS). 4 μL of Matrix (20 mg mL⁻¹ THAP and 20 mg mL⁻¹ CHCA in dry acetone) was combined with 1 μL of sample from the Cathepsin B activity assay and spotted on a stainless steel MALDI-MS plate (Bruker). After evaporation of matrix, three technical replicates were collected for each spot using FlexControl software (Bruker Daltonics) on a Bruker AutoFlex MALDI-TOF. The laser pulse rate was 1000 Hz and spectra were obtained with a mass window of 600-5000 m/z at high resolution (4.00 GS/s). FlexAnalysis software (Bruker Daltonics) was used to perform unbiased smoothing and obtain baseline spectra for all samples. Spectra were exported to Microsoft Excel and spectra were plotted using MATLAB.

Activation of STING. THP1-Dual cells (InvivoGen) were cultured in Roswell Park Memorial Institute (RPMI) 1640 Medium (Gibco) supplemented with 2 mM L-glutamine, 25 mM HEPES, 10% heat-inactivated fetal bovine serum (HI-FBS; Gibco), 100 U mL⁻¹, penicillin/100 μg mL⁻¹, streptomycin (Gibco), and 100 μg/mL Normocin. Cells were subjected to 10 μg/mL Blasticidin and 100 μg/mL Zeocin for continual selection after every cell passage. 96-well plates (REF 655180; Greiner Bio-One) were used for screening agonist activity. Reporter cells were seeded at 25,000 cells/well in 100 μL media and treatments were administered in 100 μL of medium. Results were collected 24 hours after treatment using a Quanti-Luc™ (InvivoGen) assay on cell supernatants following manufacturer's instructions. Luminescence was quantified using a plate reader (Synergy H1 Multi-Mode Microplate Reader; Biotek) after supernatants were transferred to opaque-bottom 96-well plates (REF 655073; Greiner Bio-One).

Secretion of Interferon-β (IFN-β) ELISA. Spleens were harvested from Female C57BL/6 mice (8 weeks old), mechanically disrupted into single-cell suspensions through a 70 μm cell strainer (Fisherbrand™; Thermo Fisher Scientific) and suspended in complete RPMI 1640 medium (Gibco) supplemented with 10% FBS, 10% HI-FBS (Gibco), 100 U ml⁻¹, penicillin/100 μg ml-1, streptomycin (Gibco), 50 μM 2-mercaptoethanol, and 2 mM L-glutamine. The cells were centrifuged for 5 min at 1500 rpm and resuspended in ACK lysis buffer (KD Medical) for 5 minutes. Cells were centrifuged and resuspended in fresh media at a concentration of 3 million cells per mL. Cells were seeded in a 96-well round bottom plate at 100 μL per well and treatments were administered in 100 μL of medium. Results were collected 24 hours after treatment using a mouse IFN-β solid-phase sandwich ELISA kit (Invivogen Cat #42400-1) on cell supernatants following manufacturer's instructions. Luminescence was quantified using a plate reader (Synergy H1 Multi-Mode Microplate Reader; Biotek).

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A conjugate, or a pharmaceutically acceptable salt thereof, comprising: a hydrophilic polymer; a diamidobenzimidazole (diABZI); and a linker attaching the diABZI to the hydrophilic polymer.

Clause 2. The conjugate of clause 1, or a pharmaceutically acceptable salt thereof, wherein the hydrophilic polymer comprises polyethylene glycol (PEG), a copolymer comprising a recurring hydrophilic unit and a recurring linking unit, poly(dimethylacrylamide) (PDMA), dextran, poly(lysine), poly(arginine), poly(glutamic acid), poly(aspartic acid), polyethylenimine (PEI), poly(mannose MA), poly(glucose MA), poly(galactose MA), poly(methacrylic acid), poly(propyl acrylic acid), zwitterionic polymer, poly(phosphorylcholine), poly(sulfobetaine), poly(carboxylbetaine), poly(2-methacryloyloxyethyl phosphorylcholine), or a combination thereof.

Clause 3. The conjugate of clause 1 or 2, or a pharmaceutically acceptable salt thereof, wherein the hydrophilic polymer comprises PEG, a copolymer comprising a recurring hydrophilic unit and a recurring linking unit, or PDMA.

Clause 4. The conjugate of any one of clauses 1-3, or a pharmaceutically acceptable salt thereof, wherein the hydrophilic polymer comprises PEG, the PEG having a number average molecular weight of about 1 kDa to about 50 kDa as measured by gel permeation chromatography.

Clause 5. The conjugate of any one of clauses 1-3, or a pharmaceutically acceptable salt thereof, wherein the hydrophilic polymer comprises the copolymer, the copolymer having a number average molecular weight of about 15 kDa to about 150 kDa as measured by gel permeation chromatography.

Clause 6. The conjugate of clause 3 or 5, or a pharmaceutically acceptable salt thereof, wherein the recurring hydrophilic unit of the copolymer comprises a dimethylacrylamide unit, a (2-diethylamino) ethyl methacrylate (DEAEMA) unit, a (2-dimethylamino) ethyl methacrylate (DMAEMA) unit, a N-(2-hydroxypropyl)methacrylamide unit, a 2-amino methacrylate hydrochloride unit, a polyethylene glycol methacrylate (PEGMA) unit, or a combination thereof; and at least one of the recurring linking units of the copolymer comprises the linker.

Clause 7. The conjugate of any one of clauses 1-3, or a pharmaceutically acceptable salt thereof, wherein the hydrophilic polymer comprises PDMA, the PDMA having a number average molecular weight of about 15 kDa to about 185 kDa as measured by gel permeation chromatography.

Clause 8. The conjugate of any one of clauses 1-7, or a pharmaceutically acceptable salt thereof, wherein the diABZI has formula (III)

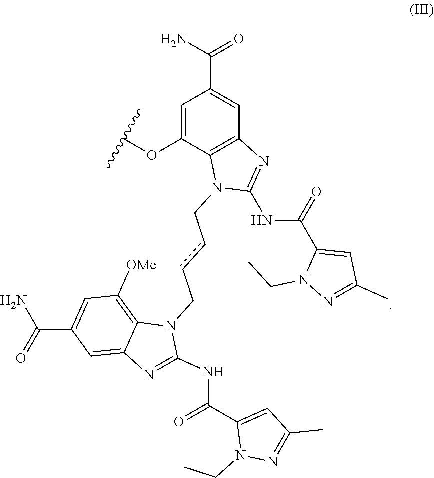

Clause 9. The conjugate of any one of clauses 1-8, or a pharmaceutically acceptable salt thereof, wherein the linker is non-cleavable.

Clause 10. The conjugate of any one of clauses 1-8, or a pharmaceutically acceptable salt thereof, wherein the linker is cleavable.

Clause 11. The conjugate of any one of clauses 1-8 or 10, or a pharmaceutically acceptable salt thereof, wherein the linker comprises a hypoxia sensitive linker, a reactive oxygen species (ROS) sensitive linker, a pH sensitive linker, a redox sensitive linker, an enzyme sensitive linker, a light sensitive linker, or a combination thereof.

Clause 12. The conjugate of clause 11, or a pharmaceutically acceptable salt thereof, wherein the enzyme sensitive linker is a matrix metalloproteinase sensitive linker, a p-aminobenzyl alcohol system linker, a cathepsin sensitive linker, a beta-glucuronidase sensitive linker, an esterase sensitive linker, or a combination thereof.

Clause 13. The conjugate of clause 11, or a pharmaceutically acceptable salt thereof, wherein the redox sensitive linker is a glutathione sensitive linker, a nitroreductase/NADH sensitive linker, or a combination thereof.

Clause 14. The conjugate of clause 11, or a pharmaceutically acceptable salt thereof, wherein the pH sensitive linker is a hydrazone, a silyl ether, a low pH sensitive linker, or a combination thereof.

Clause 15. The conjugate of any one of clauses 1-14, or a pharmaceutically acceptable salt thereof, comprising 1 to 15 diABZI per hydrophilic polymer.

Clause 16. The conjugate of any one of clauses 1-15, or a pharmaceutically acceptable salt thereof, wherein the hydrophilic polymer has a number average molecular weight of about 1 kDa to about 200 kDa as measured by gel permeation chromatography.

Clause 17. The conjugate of any one of clauses 1-16, or a pharmaceutically acceptable salt thereof, wherein the conjugate has an aqueous solubility of greater than or equal to 1 mg/ml.

Clause 18. A pharmaceutical composition comprising the conjugate of any one of clauses 1-17, or a pharmaceutically acceptable salt thereof, and a pharmaceutically acceptable excipient.

Clause 19. The pharmaceutical composition of clause 18, wherein the pharmaceutically acceptable excipient comprises saline, trehalose, sucrose, or a combination thereof.

Clause 20. A method of modulating a stimulator of interferon genes (STING) pathway in a subject in need thereof, the method comprising administering to the subject an effective amount of the conjugate according to any one of clauses 1-17, or a pharmaceutically acceptable salt thereof, optionally in combination with a pharmaceutically acceptable excipient.

Clause 21. The method of clause 20, wherein the subject has a cancer, a viral infection, or multiple sclerosis.

Clause 22. The method of clause 21, wherein the cancer is melanoma, breast cancer, neuroblastoma, renal cell carcinoma, colon cancer, lung cancer, glioma, glioblastoma, pancreatic cancer, osteosarcoma, ovarian cancer, cervical cancer, bladder cancer, T and B cell lymphomas, medulloblastoma, head and neck cancer (HNSCC), liver cancer, or prostate cancer.

Clause 23. The method of any one of clauses 20-22, wherein the method increases an IFN-β serum concentration in the subject within 1 to 4 hours after administration.

Clause 24. A method of treating cancer in a subject in need thereof, the method comprising administering to the subject an effective amount of the conjugate according to any one of clauses 1-17, or a pharmaceutically acceptable salt thereof, optionally in combination with a pharmaceutically acceptable excipient, wherein the subject has melanoma, breast cancer, neuroblastoma, renal cell carcinoma, colon cancer, lung cancer, glioma, glioblastoma, pancreatic cancer, osteosarcoma, ovarian cancer, cervical cancer, bladder cancer, T and B cell lymphomas, medulloblastoma, head and neck cancer (HNSCC), liver cancer, or prostate cancer.

Clause 25. The method of clause 24, wherein the subject has breast cancer, neuroblastoma, melanoma, renal cell carcinoma, glioblastoma, or colon cancer.

Clause 26. A compound of formula (VI), or a pharmaceutically acceptable salt thereof,

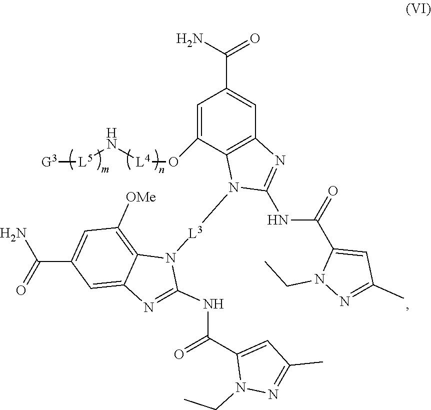

wherein: L³ is C₃₋₈alkylene or C₃₋₈alkenylene; L⁴ is C₁₋₆alkylene or C₁₋₆alkenylene; L⁵ is

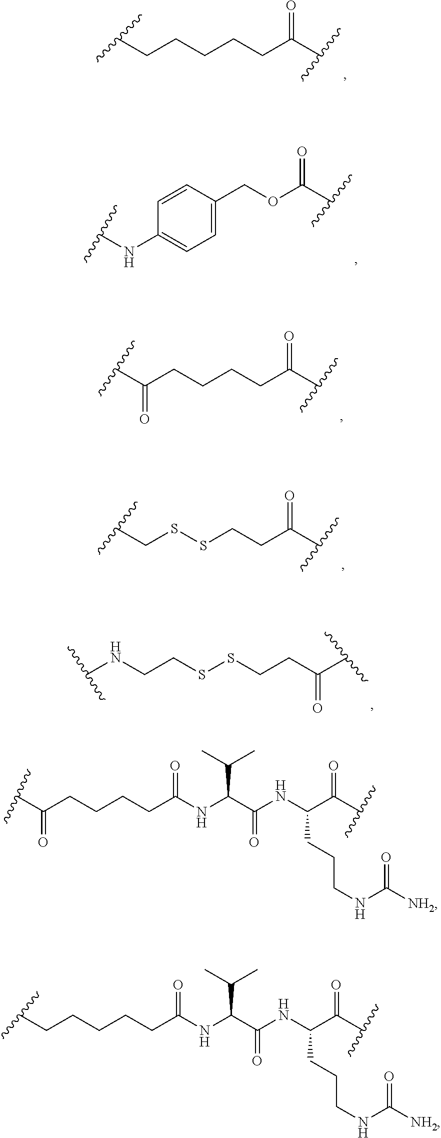

or a combination thereof; G³ is

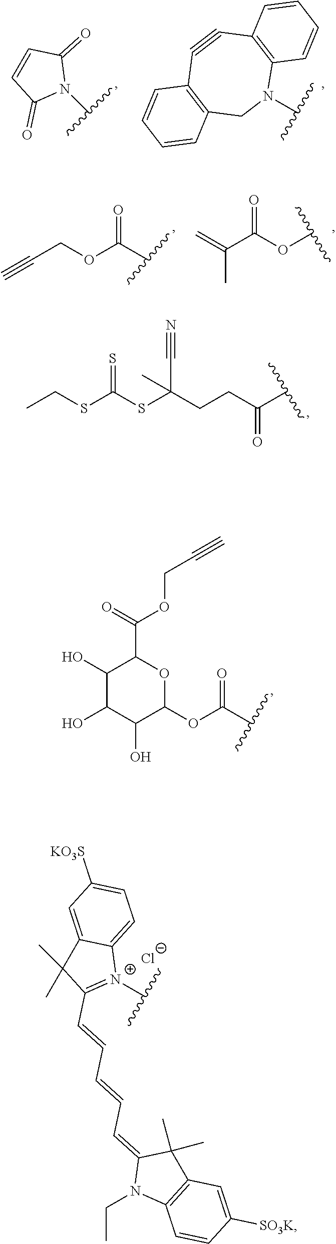

or a combination thereof; m is 0-4; and n is 1-2.

Clause 27. The compound of clause 26, or a pharmaceutically acceptable salt thereof, wherein L³ is C₃₋₈alkenylene.

Clause 28. The compound of clause 26 or 27, or a pharmaceutically acceptable salt thereof, wherein L⁴ is C₁₋₆alkylene.

Clause 29. The compound of any one of clauses 26-28, or a pharmaceutically acceptable salt thereof, wherein n is 1.

Clause 30. The compound of any one of clauses 26-29, or a pharmaceutically acceptable salt thereof, wherein m is 0, 1, 2, or 3.

Clause 31. The compound of any one of clauses 26-30, or a pharmaceutically acceptable salt thereof, wherein L⁵ is

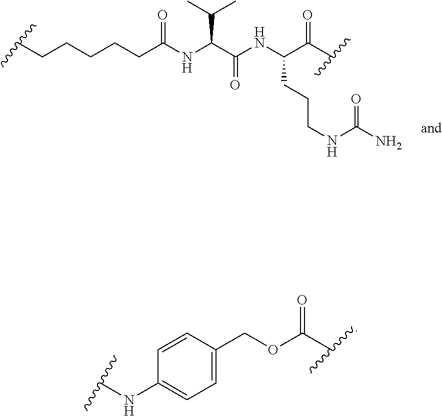 [SMILES: *CCCCCC(=O)N[C@H](C(=O)N[C@@H](CCCNC(N)=O)C(*)=O)C(C)C ; *Nc1ccc(COC(*)=O)cc1]

Clause 32. The compound of any one of clauses 26-31, or a pharmaceutically acceptable salt thereof, wherein L⁵ is

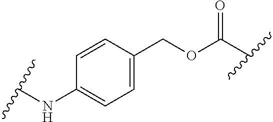 [SMILES: *Nc1ccc(COC(*)=O)cc1]

and m is 1, 2, or 3.

Clause 33. The compound of any one of clauses 26-32, or a pharmaceutically acceptable salt thereof, wherein G³ is

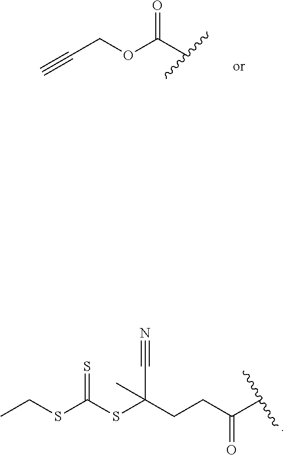

Clause 34. The compound of any one of clauses 26-33, or a pharmaceutically acceptable salt thereof, wherein the compound is of formula (VI-a):

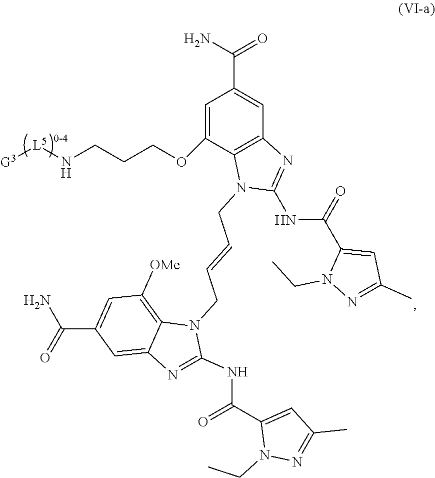

Clause 35. The compound of any one of clauses 26-34, or a pharmaceutically acceptable salt thereof, wherein L⁵ is absent.

Clause 36. The compound of any one of clauses 26-35, or pharmaceutically acceptable salt thereof, wherein L⁵ is present.

Clause 37. The compound of any one of clauses 26-33 or 35-36, or a pharmaceutically acceptable salt thereof, wherein the compound is of formula (VI-b):

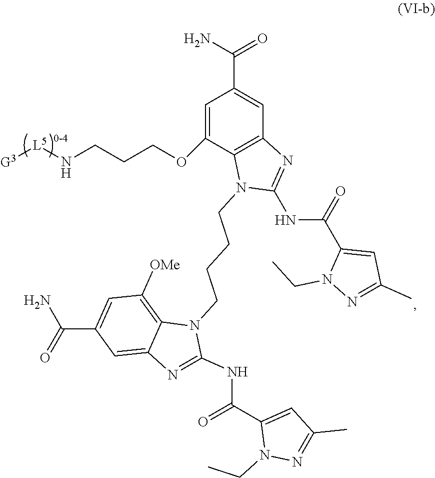

Clause 38. The compound of clause 26, selected from the group consisting of:

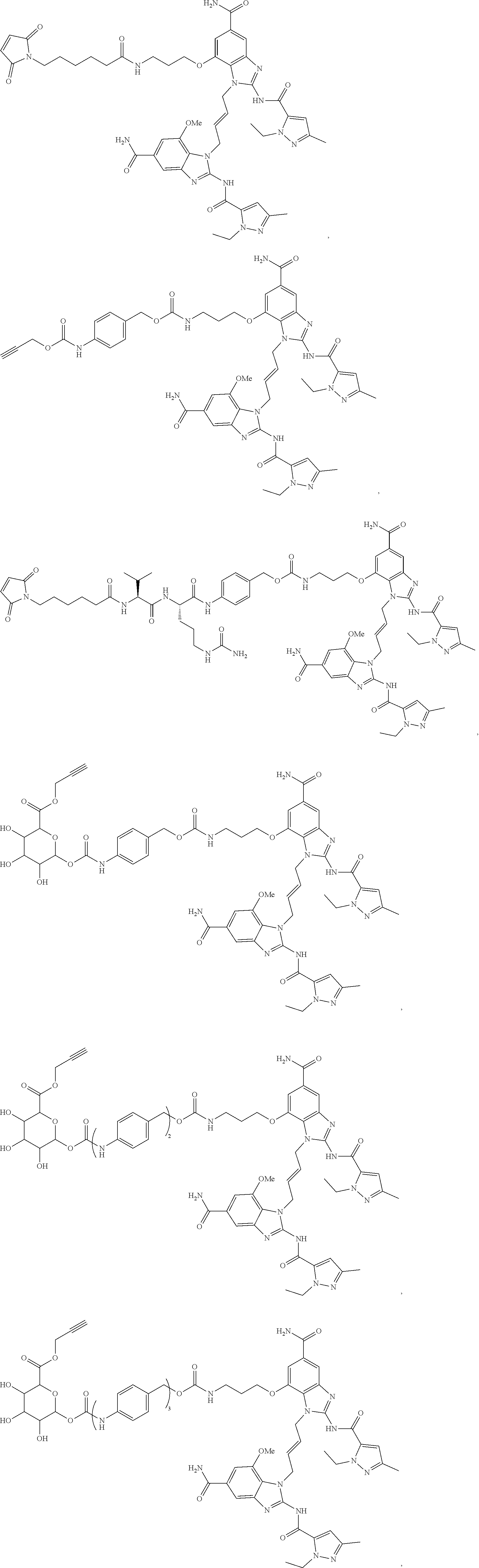

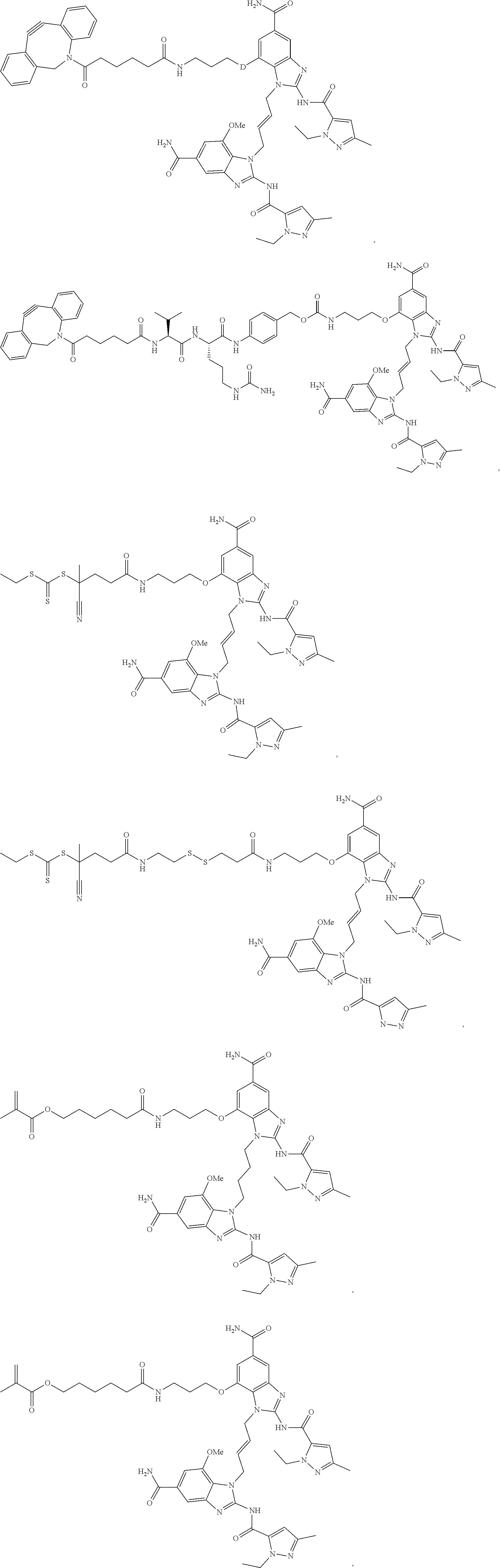

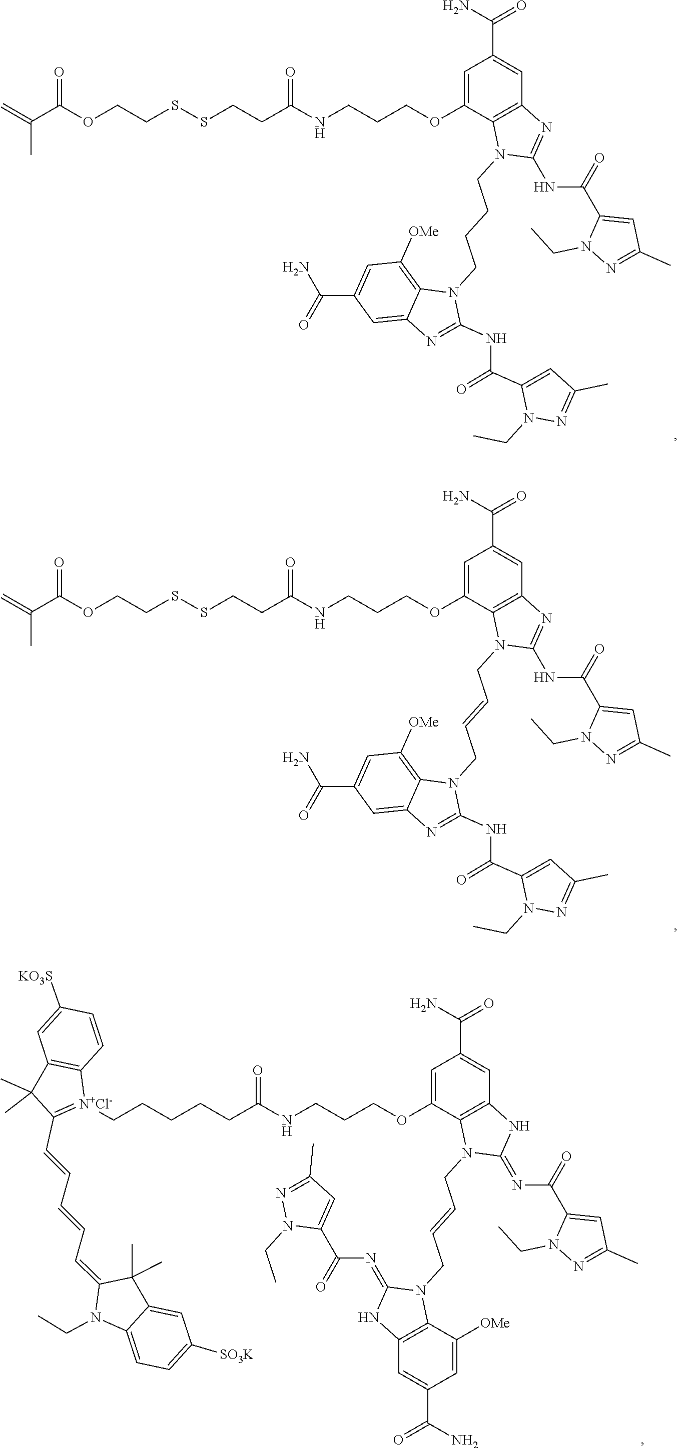

and a pharmaceutically acceptable salt thereof.

Clause 39. A pharmaceutical composition comprising the compound according to any one of clauses 26-38, or a pharmaceutically acceptable salt thereof, and a pharmaceutically acceptable carrier.

Clause 40. A method for treating a disease or disorder associated with Stimulator of Interferon Gene (STING) dysfunction comprising administering to a subject in need thereof, a therapeutically effective amount of the compound according to any one of clauses 26-38, or a pharmaceutically acceptable salt thereof, or the pharmaceutical composition of clause 39.

Clause 41. The method of clause 40, wherein the disease or disorder is cancer.

What is claimed:

1. A conjugate, or a pharmaceutically acceptable salt thereof, comprising:
a hydrophilic polymer;
a diamidobenzimidazole (diABZI); and
a linker attaching the diABZI to the hydrophilic polymer.

2. The conjugate of claim 1, or a pharmaceutically acceptable salt thereof, wherein the hydrophilic polymer comprises polyethylene glycol (PEG), a copolymer comprising a recurring hydrophilic unit and a recurring linking unit, poly(dimethylacrylamide) (PDMA), dextran, poly(lysine), poly(arginine), poly(glutamic acid), poly(aspartic acid), polyethylenimine (PEI), poly(mannose MA), poly(glucose MA), poly(galactose MA), poly(methacrylic acid), poly(propyl acrylic acid), zwitterionic polymer, poly(phosphorylcholine), poly(sulfobetaine), poly(carboxylbetaine), poly(2-methacryloyloxyethyl phosphorylcholine), or a combination thereof.

3. The conjugate of claim 1, or a pharmaceutically acceptable salt thereof, wherein the hydrophilic polymer comprises PEG, a copolymer comprising a recurring hydrophilic unit and a recurring linking unit, or PDMA.

4. The conjugate of claim 3, or a pharmaceutically acceptable salt thereof, wherein the hydrophilic polymer comprises PEG, the PEG having a number average molecular weight of about 1 kDa to about 50 kDa as measured by gel permeation chromatography.

5. The conjugate of claim 3, or a pharmaceutically acceptable salt thereof, wherein the hydrophilic polymer comprises the copolymer, the copolymer having a number average molecular weight of about 15 kDa to about 150 kDa as measured by gel permeation chromatography.

6. The conjugate of claim 3, or a pharmaceutically acceptable salt thereof, wherein the recurring hydrophilic unit of the copolymer comprises a dimethylacrylamide unit, a (2-diethylamino) ethyl methacrylate (DEAEMA) unit, a (2-dimethylamino) ethyl methacrylate (DMAEMA) unit, a N-(2-hydroxypropyl)methacrylamide unit, a 2-amino methacrylate hydrochloride unit, a polyethylene glycol methacrylate (PEGMA) unit, or a combination thereof; and at least one of the recurring linking units of the copolymer comprises the linker.

7. The conjugate of claim 3, or a pharmaceutically acceptable salt thereof, wherein the hydrophilic polymer comprises PDMA, the PDMA having a number average molecular weight of about 15 kDa to about 185 kDa as measured by gel permeation chromatography.

8. The conjugate of claim 1, or a pharmaceutically acceptable salt thereof, wherein the diABZI has formula (III)

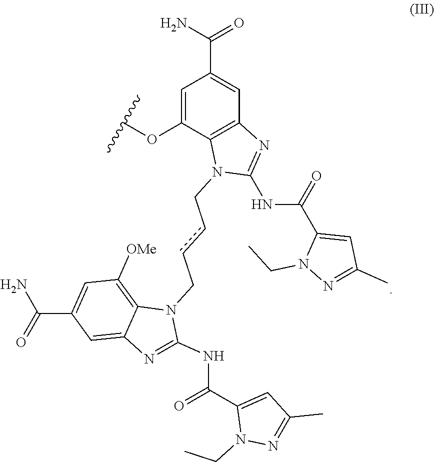

9. The conjugate of claim 1, or a pharmaceutically acceptable salt thereof, wherein the linker is non-cleavable.

10. The conjugate of claim 1, or a pharmaceutically acceptable salt thereof, wherein the linker is cleavable.

11. The conjugate of claim 1, or a pharmaceutically acceptable salt thereof, wherein the linker comprises a hypoxia sensitive linker, a reactive oxygen species (ROS) sensitive linker, a pH sensitive linker, a redox sensitive linker, an enzyme sensitive linker, a light sensitive linker, or a combination thereof.

12. The conjugate of claim 11, or a pharmaceutically acceptable salt thereof, wherein the enzyme sensitive linker is a matrix metalloproteinase sensitive linker, a p-aminobenzyl alcohol system linker, a cathepsin sensitive linker, a beta-glucuronidase sensitive linker, an esterase sensitive linker, or a combination thereof.

13. The conjugate of claim 11, or a pharmaceutically acceptable salt thereof, wherein the redox sensitive linker is a glutathione sensitive linker, a nitroreductase/NADH sensitive linker, or a combination thereof.

14. The conjugate of claim 11, or a pharmaceutically acceptable salt thereof, wherein the pH sensitive linker is a hydrazone, a silyl ether, a low pH sensitive linker, or a combination thereof.

15. The conjugate of claim 1, or a pharmaceutically acceptable salt thereof, comprising 1 to 15 diABZI per hydrophilic polymer.

16. The conjugate of claim 1, or a pharmaceutically acceptable salt thereof, wherein the hydrophilic polymer has a number average molecular weight of about 1 kDa to about 200 kDa as measured by gel permeation chromatography.

17. The conjugate of claim 1, or a pharmaceutically acceptable salt thereof, wherein the conjugate has an aqueous solubility of greater than or equal to 1 mg/ml.

18. A pharmaceutical composition comprising the conjugate of claim 1, or a pharmaceutically acceptable salt thereof, and a pharmaceutically acceptable excipient.

19. The pharmaceutical composition of claim 18, wherein the pharmaceutically acceptable excipient comprises saline, trehalose, sucrose, or a combination thereof.

20. A method of modulating a stimulator of interferon genes (STING) pathway in a subject in need thereof, the method comprising administering to the subject an effective amount of the conjugate according to claim 1, or a pharmaceutically acceptable salt thereof, optionally in combination with a pharmaceutically acceptable excipient.

21. The method of claim 20, wherein the subject has a cancer, a viral infection, or multiple sclerosis.

22. The method of claim 21, wherein the cancer is melanoma, breast cancer, neuroblastoma, renal cell carcinoma, colon cancer, lung cancer, glioma, glioblastoma, pancreatic cancer, osteosarcoma, ovarian cancer, cervical cancer, bladder cancer, T and B cell lymphomas, medulloblastoma, head and neck cancer (HNSCC), liver cancer, or prostate cancer.

23. The method of claim 20, wherein the method increases an IFN-βserum concentration in the subject within 1 to 4 hours after administration.

24. A method of treating cancer in a subject in need thereof, the method comprising administering to the subject an effective amount of the conjugate according to claim 1, or a pharmaceutically acceptable salt thereof, optionally in combination with a pharmaceutically acceptable excipient, wherein the subject has melanoma, breast cancer, neuroblastoma, renal cell carcinoma, colon cancer, lung cancer, glioma, glioblastoma, pancreatic cancer, osteosarcoma, ovarian cancer, cervical cancer, bladder cancer, T and B cell lymphomas, medulloblastoma, head and neck cancer (HNSCC), liver cancer, or prostate cancer.

25. The method of claim 24, wherein the subject has breast cancer, neuroblastoma, melanoma, renal cell carcinoma, glioblastoma, or colon cancer.

26. A compound of formula (VI), or a pharmaceutically acceptable salt thereof, 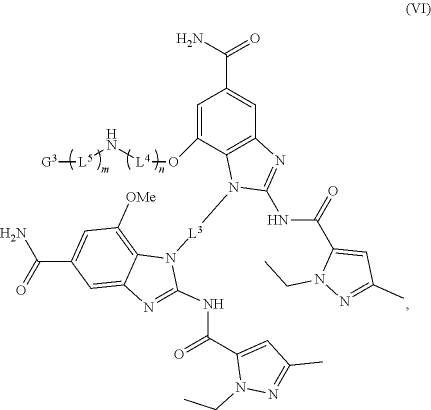
wherein:
L³ is C₃₋₈alkylene or C₃₋₈alkenylene;
L⁴ is C₁₋₆alkylene or C₁₋₆alkenylene;
L⁵ is 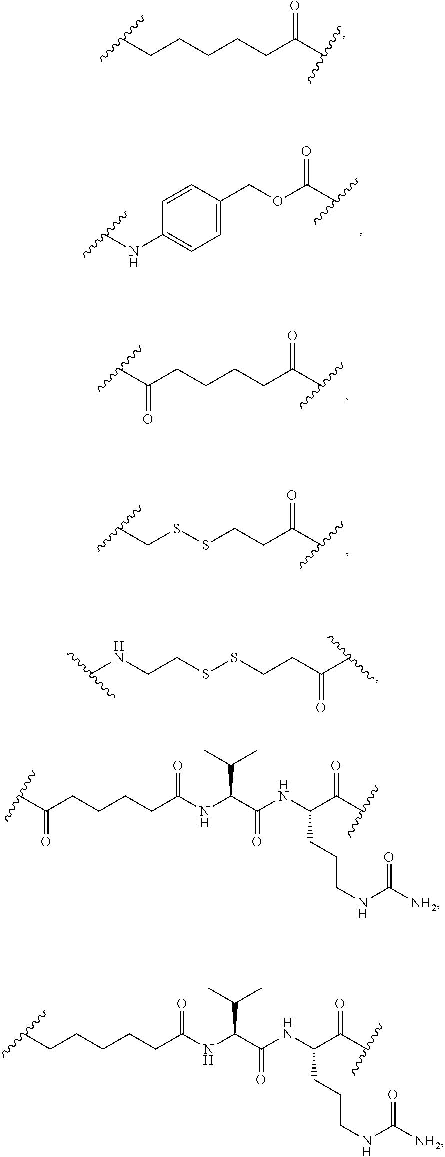
or a combination thereof;
G¹ is 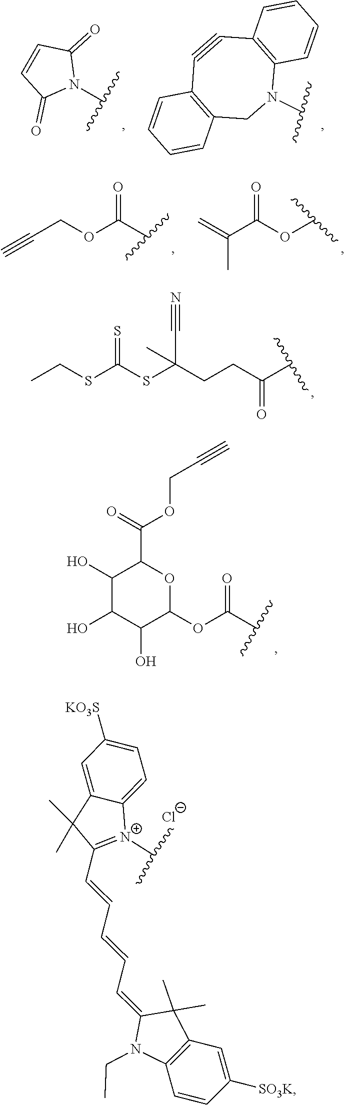
or a combination thereof;
m is 0-4; and
n is 1-2.

27. The compound of claim 26, or a pharmaceutically acceptable salt thereof, wherein L³ is C₃₋₈alkenylene.

28. The compound of claim 26, or a pharmaceutically acceptable salt thereof, wherein L⁴ is C₁₋₆alkylene.

29. The compound of claim 26, or a pharmaceutically acceptable salt thereof, wherein n is 1.

30. The compound of claim 26, or a pharmaceutically acceptable salt thereof, wherein m is 0, 1, 2, or 3.

31. The compound of claim 26, or a pharmaceutically acceptable salt thereof, wherein L⁵ is

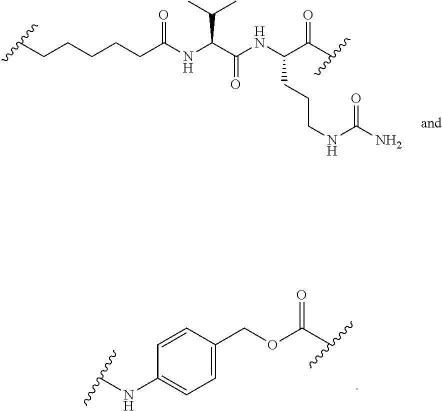 [SMILES: *CCCCCC(=O)N[C@H](C(=O)N[C@@H](CCCNC(N)=O)C(*)=O)C(C)C ; *Nc1ccc(COC(*)=O)cc1]

32. The compound of claim 26, or a pharmaceutically acceptable salt thereof, wherein L⁵ is 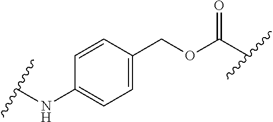 [SMILES: *Nc1ccc(COC(*)=O)cc1]
and m is 1, 2, or 3.

33. The compound of claim 26, or a pharmaceutically acceptable salt thereof, wherein G³ is

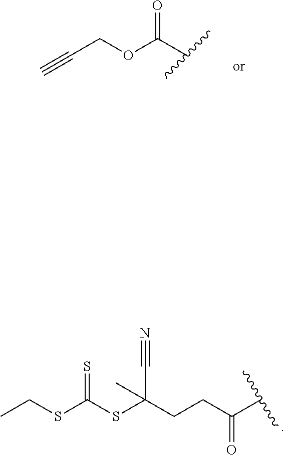

34. The compound of claim 26, or a pharmaceutically acceptable salt thereof, wherein the compound is of formula (VI-a):

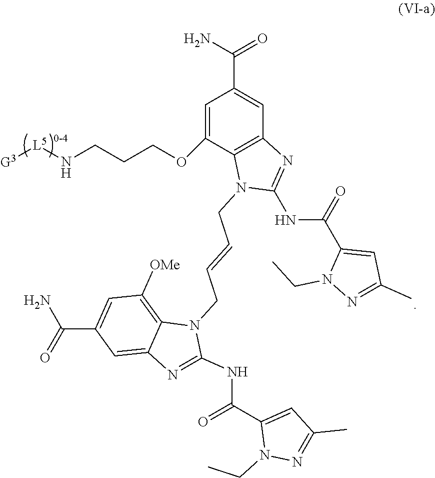

35. The compound of claim 26, or a pharmaceutically acceptable salt thereof, wherein L⁵ is absent.

36. The compound of claim 26, or pharmaceutically acceptable salt thereof, wherein L⁵ is present.

37. The compound of claim 26, or a pharmaceutically acceptable salt thereof, wherein the compound is of formula (VI-b):

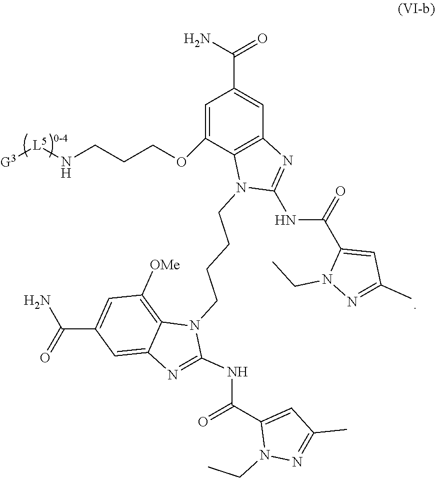

38. The compound of claim 26, selected from the group consisting of: 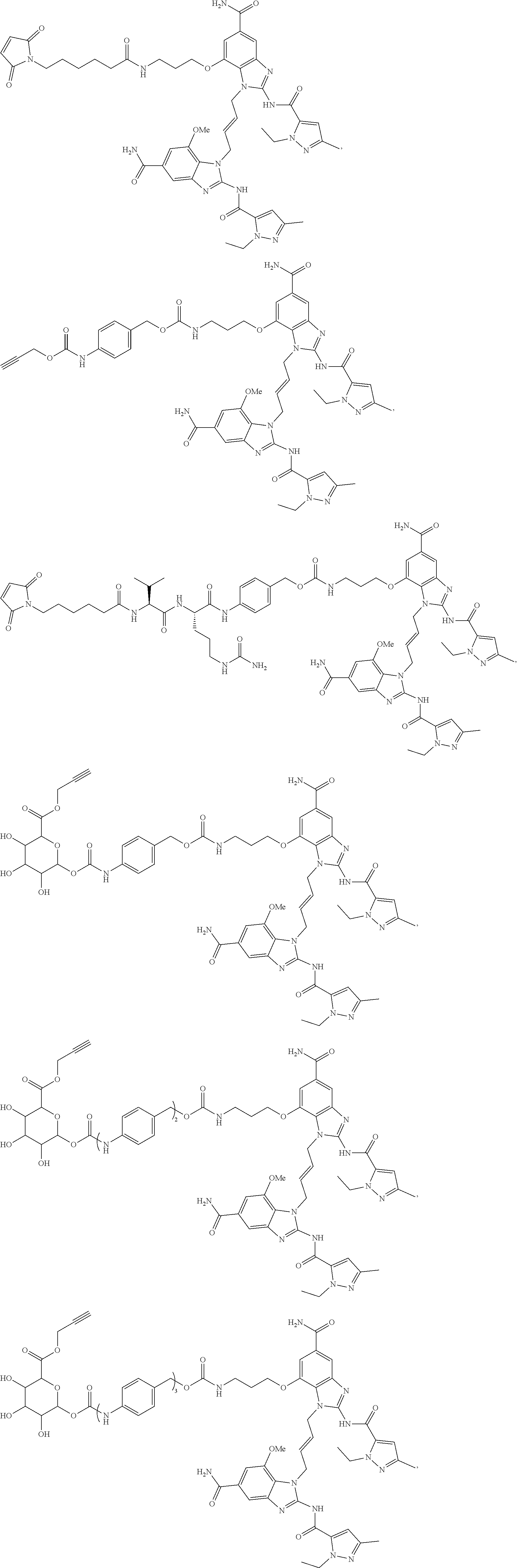 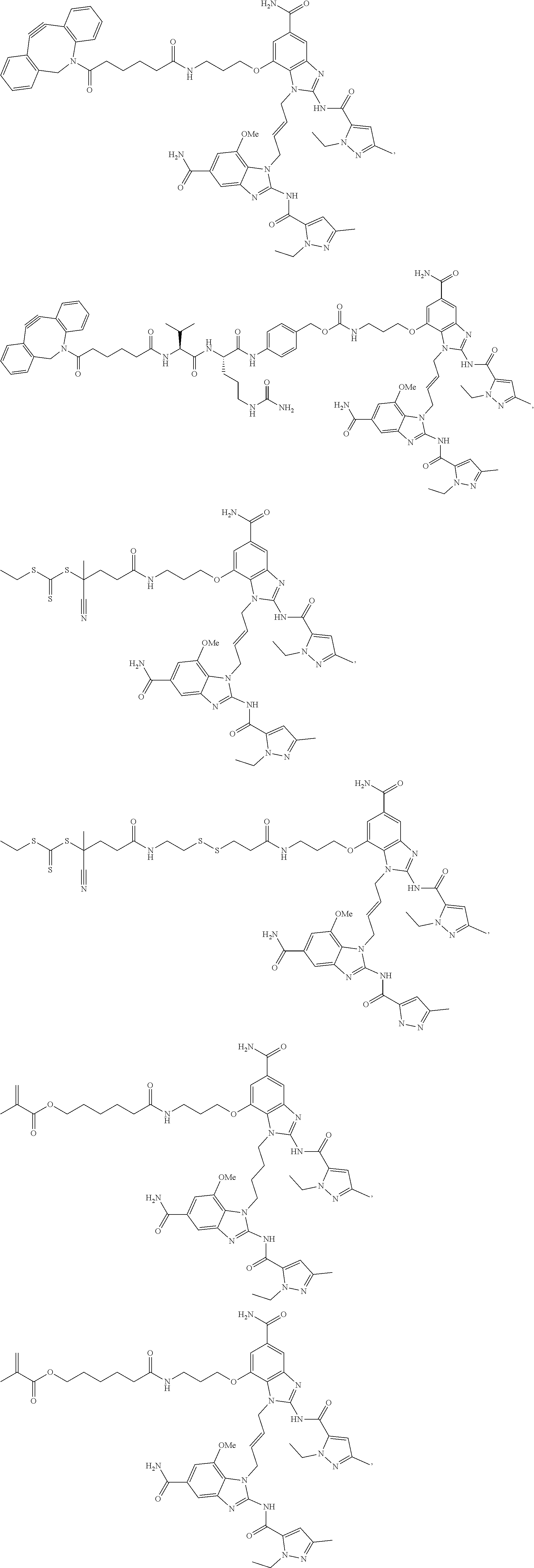 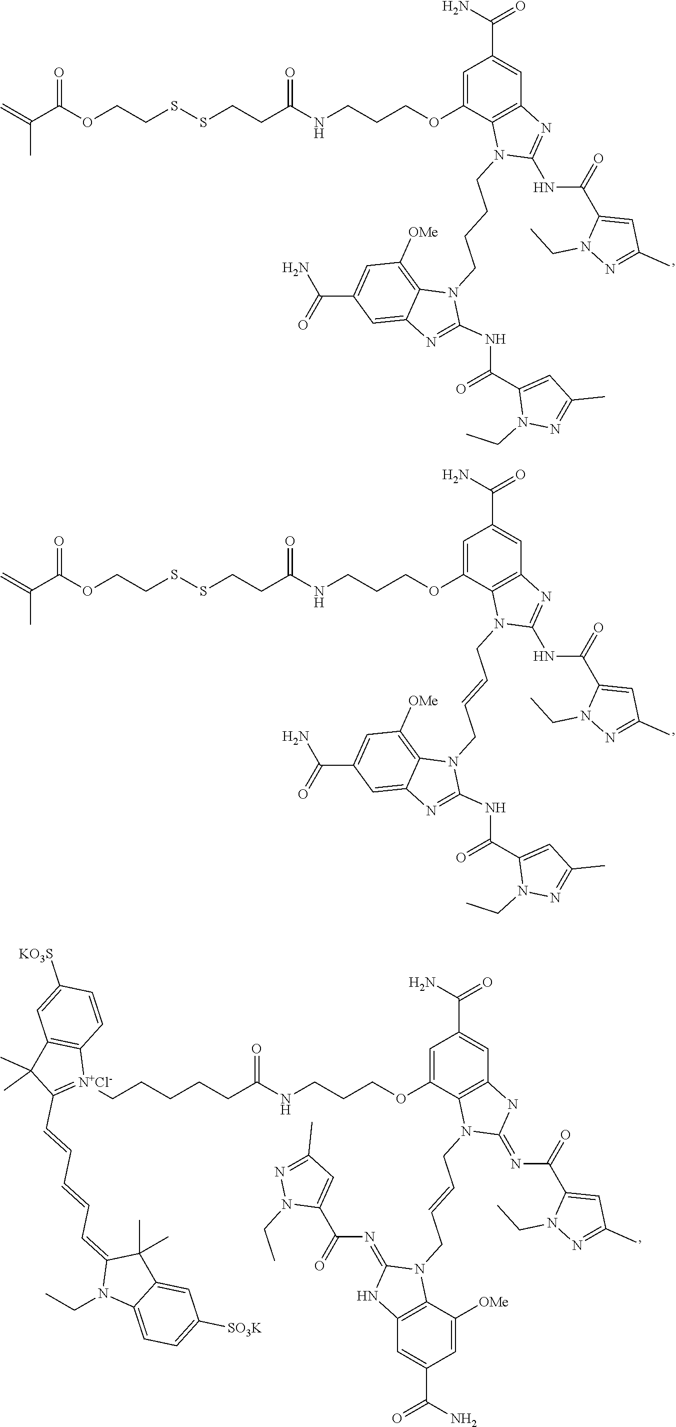
and a pharmaceutically acceptable salt thereof.

39. A pharmaceutical composition comprising the compound of claim 26, or a pharmaceutically acceptable salt thereof, and a pharmaceutically acceptable carrier.

40. A method for treating a disease or disorder associated with Stimulator of Interferon Gene (STING) dysfunction comprising administering to a subject in need thereof, a therapeutically effective amount of the compound of claim 26, or a pharmaceutically acceptable salt thereof, or the pharmaceutical composition of claim 39.

41. The method of claim 40, wherein the disease or disorder is cancer.
